(19) United States
(12) Patent Application Publication
(10) Pub. No.: US 2021/0139441 A1
(43) Pub. Date: May 13, 2021

(54) USES OF FLUORINATED EPOXIDES AND NOVEL MIXTURES THEREOF

(71) Applicant: THE CHEMOURS COMPANY FC, LLC, WILMINGTON, DE (US)

(72) Inventors: Viacheslav A. Petrov, Hockessin, DE (US); Drew Richard Brandt, West Chester, PA (US); Barbara Haviland Minor, Elkton, MD (US); Konstantinos Kontomaris, Wilmington, DE (US); Mark L Robin, Middletown, DE (US); Emest Byron Wysong, Chadds Ford, PA (US); Harrison K Musyimi, Bear, DE (US); Christine M Glatt, Gamet Valley, PA (US)

(73) Assignee: THE CHEMOURS COMPANY FC, LLC, WILMINGTON, DE (US)

(21) Appl. No.: 16/492,188

(22) PCT Filed: Mar. 9, 2018
(86) PCT No.: PCT/US18/21864

§ 371 Date: Sep. 9, 2019

Related U.S. Application Data
(60) Provisional application No. 62/469,679, filed on Mar. 10, 2017.

(51) Int. Cl. C07D 303/48; A62D 1/00; C07D 303/08; C07D 303/22; C09K 5/04

(52) U.S. Cl. CPC C07D 303/48; A62D 1/0057; C07D 303/08; C09K 2205/116; C09K 5/045; C09K 2205/40; C07D 303/22

(57) ABSTRACT

This application relates to compositions comprising one or more partially fluorinated epoxides and/or one or more perfluorinated epoxides which may be useful in applications including refrigerants, air conditioning, heat transfer media, high-temperature heat pumps, organic Rankine cycles, fire extinguishing/fire suppression, propellants, foam blowing, solvents, dielectrics, and/or cleaning fluids.

### CROSS-REFERENCE TO RELATED APPLICATIONS

This application claims the benefit of U.S. Provisional Application Ser. No. 62/469,679, filed Mar. 10, 2017, the disclosure of which is incorporated herein by reference in its entirety.

### TECHNICAL FIELD

This invention relates to compositions comprising one or more partially fluorinated epoxides and/or one or more perfluorinated epoxides which may be useful in applications including refrigerants, air conditioning, heat transfer media, high-temperature heat pumps, organic Rankine cycles, fire extinguishing/fire suppression, propellants, foam blowing, solvents, dielectrics, and/or cleaning fluids.

### BACKGROUND

Many current commercial propellant, fire suppression and foam blowing applications employ HCFCs or HFCs. HCFCs, due to their Cl content, contribute to ozone depletion and are scheduled for eventual phaseout under the Montreal Protocol. HFCs, while not contributing to ozone depletion, can contribute to global warming, and the use of such compounds has come under scrutiny by environmental regulators. Thus, there is a need for aerosol propellants, fire suppression agents, foam blowing agents, refrigerants, solvents, cleaning and cleaning fluids that are characterized by a zero ozone depletion potential (ODP) and low impact on global warming. This application addresses this need and others.

### SUMMARY

The present application provides, inter alia, a composition for use in refrigeration, in air conditioning, in heating, in heat transfer, in conversion of heat into mechanical work in a power cycle, as a foam blowing agent, as a solvent, or in preventing or quenching an electric discharge, comprising a refrigerant component, an air conditioning component, a heating component, a heat transfer component, a working fluid component, a blowing agent component, a solvent component, or a dielectric component, respectively, which is a compound of Formula (I):

[chemical structure]

wherein R¹, R², R³, and R⁴ are defined infra in various embodiments.

Accordingly, the present application also provides compositions for use in refrigeration, wherein the composition comprises a refrigerant component which is a compound of Formula (I).

The present application further provides compositions for use in air conditioning, wherein the composition comprises an air conditioning component which is a compound of Formula (I).

The present application also provides processes for producing cooling, comprising evaporating the refrigerant component in a composition described herein in the vicinity of a body to be cooled, and thereafter condensing said refrigerant component.

The present application also provides processes for replacing an incumbent refrigerants, comprising substantially replacing the incumbent refrigerant with a composition described herein.

The present application further provides compositions for use in heating, wherein the composition comprises a heating component, which is a compound of Formula (I).

The present application also provides processes for producing heating, comprising condensing a composition described herein in the vicinity of a body to be heated, and thereafter evaporating the heating component.

The present application further provides compositions for use in heat transfer, wherein the working fluid component is a heat transfer component, which is a compound of Formula (I).

The present application also provides processes for transferring heat from heat source to heat sink, comprising transporting a composition described herein from the heat source to the heat sink.

The present application further provides compositions for conversion of heat into mechanical work in a power cycle, wherein the composition comprises a working fluid component, which is a compound of Formula (I).

The present application also provides a process for converting heat into mechanical work in a power cycle, comprising the steps of heating a composition of described herein with a heat source to a temperature sufficient to pressurize the composition; and causing the pressurized composition to perform mechanical work.

The present application further provides compositions for use as a foam blowing agent, wherein the composition comprises a blowing agent component, which is a compound of Formula (I) as described herein.

The present application also provides a foamable composition comprising the foam blowing agent compositions as described herein and one or more additional components capable of reacting and/or foaming under the proper conditions to form a foam or cellular structure.

The present application also provides processes for forming a foam, comprising reacting or extruding a foamable composition described herein under conditions effective to form a foam.

The present application further provides compositions for use as a solvent, wherein the composition comprises the solvent component, which is a compound of Formula (I).

The present application also provides processes for dissolving a solute, comprising contacting and mixing said solute with a sufficient quantity of a composition according described herein.

The present application also provides processes of cleaning a surface, comprising contacting a composition described herein.

The present application also provides processes for removing at least a portion of water from the surface of a wetted substrate, comprising contacting the substrate with a composition described herein and then removing the substrate from contact with the composition.

The present application also provides processes for depositing a coating on a surface, comprising contacting a composition described herein with said surface, wherein the composition further comprises a depositable material.

The present application further provides compositions for use in preventing or rapidly quenching an electric discharge, wherein the composition comprises a dielectric component, which is a compound of Formula (I).

The present application also provides methods for preventing or rapidly quenching an electric discharge in a space in a high voltage device comprising injecting a gaseous dielectric into said space, wherein said gaseous dielectric comprises a composition described herein.

The present application further provides compositions for fire suppression or fire extinguishment, comprising (a) a fluoroepoxide selected from (Z)-2,3-difluoro-2-(trifluoromethyl)oxirane, (E)-2,3-difluoro-2-(trifluoromethyl)oxirane, trans-2-fluoro-3-(trifluoromethyl)oxirane, trans-2,3-bis(trifluoromethyl)oxirane, trans-2-(trifluoromethyl)-3-(perfluoroethyl)oxirane, cis-2-fluoro-3-(trifluoromethyl)oxirane, trans-2-fluoro-3-(perfluoropropan-2-yl)oxirane, or a mixture thereof; and (b) one or more of 2-bromo-1,1,1-trifluoro-2-propene, E-1,2-dichloro-1,2-difluoroethylene, Z-1,2-dichloro-1,2-difluoroethylene, E-1-chloro-3,3,3-trifluoropropene, Z-1-chloro-3,3,3-trifluoropropene, E-1,1,1,4,4,4-hexafluoro-2-butene, Z-1,1,1,4,4,4-hexafluoro-2-butene, perfluoroethyl perfluoroisopropyl ketone (F-ethyl isopropyl ketone), E-1,3,4,4,4-pentafluoro-3-(trifluoromethyl)-1-butene), E-1,2,3,3,3-pentafluoropropene, Z-1,2,3,3,3-pentafluoropropene, E-1-chloro-2,3,3,3-tetrafluoropropene, Z-1-chloro-2,3,3,3-tetrafluoropropene, CF3I, carbon dioxide, nitrogen, and argon.

The present application further provides processes for extinguishing or suppressing a flame comprising dispensing a composition described herein at the flame.

The present application further provides sprayable compositions comprising a propellant component and a co-propellant component, which is a compound of Formula (I).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DETAILED DESCRIPTION

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

### Definitions, Abbreviations, and Acronyms

For the terms “for example” and “such as” and grammatical equivalences thereof, the phrase “and without limitation” is understood to follow unless explicitly stated otherwise. As used herein, the term “about” is meant to account for variations due to experimental error. All measurements reported herein are understood to be modified by the term “about”, whether or not the term is explicitly used, unless explicitly stated otherwise. As used herein, the singular forms “a,” “an,” and “the” include plural referents unless the context clearly dictates otherwise.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

As used herein, the term “substantially isolated” is means that the compound is at least partially or substantially separated from the environment in which it was formed or detected.

Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds are routine in the art.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the term “Cₙ₋ₘ alkyl” refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group has 1 to 20, 1 to 18, 1 to 15, 1 to 12, 1 to 10, 1 to 6, 1 to 4, 1 to 3, or 1 to 2 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 1 to 20, 1 to 18, 1 to 15, 1 to 12, 1 to 10, 1 to 6, 1 to 4, 1 to 3, or 1 to 2 carbon atoms.

As used herein, “halo” refers to F, Cl, Br, or I. In some embodiments, a halo is F, Cl, or Br.

As used herein, “halide” refers to fluoride, chloride, bromide, or iodide. In some embodiments, a halide is chloride or bromide.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only (i.e. a partially fluorinated alkoxy or a perfluorinated alkoxy). In some embodiments, the haloalkoxy group has 1 to 20, 1 to 18, 1 to 15, 1 to 12, 1 to 10, 1 to 6, 1 to 4, 1 to 3, or 1 to 2 carbon atoms.

As used herein, the term “Cₙ₋ₘ haloalkyl” refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only (i.e., a partially fluorinated alkyl or a perfluorinated alkyl). In some embodiments, the haloalkyl group has 1 to 20, 1 to 18, 1 to 15, 1 to 12, 1 to 10, 1 to 6, 1 to 4, 1 to 3, or 1 to 2 carbon atoms.

As used herein, the term “partially fluorinated Cₙ₋ₘ alkyl” refers to a linear or branched alkyl group having from one halogen atom to less than 2s+1 halogen atoms which may be the same or different, where “s” is the number of carbon atoms in the alkyl group, and wherein the alkyl group has n to m carbon atoms. Examples of partially fluorinated Cₙ₋ₘ alkyl groups include, but are not limited to, —CH₂F, —CHF₂, —CH₂CH₂F, —CH₂CHF₂, —CH₂CF₃, —CH₂CH₂CF₃, —CH₂CF₂CF₃, —CF₂CF₂CHF₂, and the like. In some embodiments, the partially fluorinated alkyl group has 1 to 20, 1 to 18, 1 to 15, 1 to 12, 1 to 10, 1 to 6, 1 to 4, 1 to 3, or 1 to 2 carbon atoms.

As used herein, the term “perfluorinated Cₙ₋ₘ alkyl” refers to a linear or branched alkyl group having 2s+1 fluorine atoms, where “s” is the number of carbon atoms in the alkyl group, and wherein the alkyl group has n to m carbon atoms. Examples of perfluorinated alkyl groups include, but are not limited to, —CF₃, —CF₂CF₃, —CF₂CF₂CF₃, —CF₂CF₂CF₂CF₃, —C(F)(CF₃)₂, and the like. In some embodiments, the perfluorinated alkyl group has 1 to 20, 1 to 18, 1 to 15, 1 to 12, 1 to 10, 1 to 6, 1 to 4, 1 to 3, or 1 to 2 carbon atoms.

As used herein, the term “partially fluorinated Cₙ₋ₘ alkoxy” refers to a group of formula —O-fluoroalkyl, wherein the fluoroalkyl is a linear or branched partially fluorinated alkyl group having n to m carbon atoms. Examples of partially fluorinated alkoxy groups include, but are not limited to, —OCH₂F, —OCHF₂, —OCH₂CH₂F, —OCH₂CHF₂, —OCH₂CF₃, —OCH₂CH₂CF₃, —OCH₂CF₂CF₃, —OCF₂CF₂CHF₂, and the like. In some embodiments, the partially fluorinated alkoxy group has 1 to 20, 1 to 18, 1 to 15, 1 to 12, 1 to 10, 1 to 6, 1 to 4, 1 to 3, or 1 to 2 carbon atoms.

As used herein, the term “perfluorinated Cₙ₋ₘ alkyl” refers to a group of formula —O— fluoroalkyl, wherein the fluoroalkyl group is a linear or branched perfluoroalkyl group having n to m carbon atoms. Examples of perfluorinated alkyl groups include, but are not limited to, —CF₃, —CF₂CF₃, —CF₂CF₂CF₃, —CF₂CF₂CF₂CF₃, —C(F)(CF₃)₂, and the like. In some embodiments, the perfluorinated alkyl group has 1 to 20, 1 to 18, 1 to 15, 1 to 12, 1 to 10, 1 to 6, 1 to 4, 1 to 3, or 1 to 2 carbon atoms.

At certain places, the definitions or embodiments refer to specific rings (e.g., an azetidine ring, a pyridine ring, etc.). Unless otherwise indicated, these rings can be attached to any ring member provided that the valency of the atom is not exceeded.

As used herein, the term “azeotropic composition” shall be understood to mean a composition where at a given temperature at equilibrium, the boiling point pressure (of the liquid phase) is identical to the dew point pressure (of the vapor phase), i.e., X2═Y2. One way to characterize an azeotropic composition is that the vapor produced by partial evaporation or distillation of the liquid has the same composition as the liquid from which it was evaporated or distilled, that is, the admixture distills/refluxes without compositional change. Constant boiling compositions are characterized as azeotropic because they exhibit either a maximum or minimum boiling point, as compared with that of the non-azeotropic mixtures of the same components. Azeotropic compositions are also characterized by a minimum or a maximum in the vapor pressure of the mixture relative to the vapor pressure of the neat components at a constant temperature.

As used herein, the terms “azeotrope-like composition” and “near-azeotropic composition” shall be understood to mean a composition wherein the difference between the bubble point pressure (“BP”) and dew point pressure (“DP”) of the composition at a particular temperature is less than or equal to 5 percent based upon the bubble point pressure, i.e., [(BP-DP)/BP]×100≤5. As used herein, the terms “3 percent azeotrope-like composition” and “3 percent near-azeotropic composition” shall be understood to mean a composition wherein the difference between the bubble point pressure (“BP”) and dew point pressure (“DP”) of the composition at a particular temperature is less than or equal to 3 percent based upon the bubble point pressure, i.e., [(BP-DP)/BP]×100≤3.

Global warming potential (GWP) is an index for estimating relative global warming contribution due to atmospheric emission of a kilogram of a particular greenhouse gas compared to emission of a kilogram of carbon dioxide. GWP can be calculated for different time horizons showing the effect of atmospheric lifetime for a given gas. The GWP for the 100 year time horizon is commonly the value referenced.

As used herein the term “Ozone depletion potential” (ODP) is defined in “The Scientific Assessment of Ozone Depletion, 2002, A report of the World Meteorological Association's Global Ozone Research and Monitoring Project,” section 1.4.4, pages 1.28 to 1.31 (see first paragraph of this section). ODP represents the extent of ozone depletion in the stratosphere expected from a compound on a mass-for-mass basis relative to fluorotrichloromethane (CFC-11).

Refrigeration capacity (sometimes referred to as cooling capacity) is a term to define the change in enthalpy of a refrigerant or working fluid in an evaporator per unit mass of refrigerant or working fluid circulated. Volumetric cooling capacity refers to the amount of heat removed by the refrigerant or working fluid in the evaporator per unit volume of refrigerant vapor exiting the evaporator. The refrigeration capacity is a measure of the ability of a refrigerant, working fluid or heat transfer composition to produce cooling. Therefore, the higher the volumetric cooling capacity of the working fluid, the greater the cooling rate that can be produced at the evaporator with the maximum volumetric flow rate achievable with a given compressor. Cooling rate refers to the heat removed by the refrigerant in the evaporator per unit time.

Similarly, volumetric heating capacity is a term to define the amount of heat supplied by the refrigerant or working fluid in the condenser per unit volume of refrigerant or working fluid vapor entering the compressor. The higher the volumetric heating capacity of the refrigerant or working fluid, the greater the heating rate that is produced at the condenser with the maximum volumetric flow rate achievable with a given compressor.

Coefficient of performance (COP) is the amount of heat removed in the evaporator divided by the energy required to operate the compressor. The higher the COP, the higher the energy efficiency. COP is directly related to the energy efficiency ratio (EER), that is, the efficiency rating for refrigeration or air conditioning equipment at a specific set of internal and external temperatures.

As used herein, a heat transfer medium comprises a composition used to carry heat from a heat source to a heat sink. For example, heat from a body to be cooled to a chiller evaporator or from a chiller condenser to a cooling tower or other configuration where heat can be rejected to the ambient.

As used herein, a working fluid or refrigerant comprises a compound or mixture of compounds that function to transfer heat in a cycle wherein the working fluid undergoes a phase change from a liquid to a gas and back to a liquid in a repeating cycle.

Subcooling is the reduction of the temperature of a liquid below that liquid's saturation point for a given pressure. The saturation point is the temperature at which a vapor composition is completely condensed to a liquid (also referred to as the bubble point). But subcooling continues to cool the liquid to a lower temperature liquid at the given pressure. By cooling a liquid below the saturation temperature, the net refrigeration capacity can be increased. Subcooling thereby improves refrigeration capacity and energy efficiency of a system. Subcool amount is the amount of cooling below the saturation temperature (in degrees) or how far below its saturation temperature a liquid composition is cooled.

Superheat is a term that defines how far above the saturation vapor temperature of a vapor composition a vapor composition is heated. Saturation vapor temperature is the temperature at which, if a vapor composition is cooled, the first drop of liquid is formed, also referred to as the “dew point”.

As used herein, the term “incumbent refrigerant” shall be understood to mean the refrigerant for which the heat transfer system was designed to operate, or the refrigerant that is resident in the heat transfer system.

By “in the vicinity of” is meant that the evaporator of the system containing the refrigerant composition is located either within or adjacent to the body to be cooled, such that air moving over the evaporator would move into or around the body to be cooled. In the process for producing heating, “in the vicinity of” means that the condenser of the system containing the refrigerant composition is located either within or adjacent to the body to be heated, such that the air moving over the evaporator would move into or around the body to be heated.

The term “extinguishment” is usually used to denote complete elimination of a fire; whereas, “suppression” is often used to denote reduction, but not necessarily total elimination, of a fire or explosion. As used herein, terms “extinguishment” and “suppression” will be used interchangeably.

As used herein, the term “lubricant” refers to any material added to a composition or a compressor (and in contact with any heat transfer composition in use within any heat transfer system) that provides lubrication to the compressor to aid in preventing parts from seizing.

As used herein, the term “compatibilizers” refers to compounds which improve solubility of the hydrofluorocarbon of the disclosed compositions in heat transfer system lubricants. In some embodiments, the compatibilizers improve oil return to the compressor. In some embodiments, the composition is used with a system lubricant to reduce oil-rich phase viscosity.

As used herein, “ultra-violet” dye is defined as a UV fluorescent or phosphorescent composition that absorbs light in the ultra-violet or “near” ultra-violet region of the electromagnetic spectrum. The fluorescence produced by the UV fluorescent dye under illumination by a UV light that emits at least some radiation with a wavelength in the range of from 10 nanometers to about 775 nanometers may be detected.

Flammability is a term used to mean the ability of a composition to ignite and/or propagate a flame. For refrigerants and other heat transfer compositions, the lower flammability limit (“LFL”) is the minimum concentration of the heat transfer composition in air that is capable of propagating a flame through a homogeneous mixture of the composition and air under test conditions specified in ASTM (American Society of Testing and Materials) E681. The upper flammability limit (“UFL”) is the maximum concentration of the heat transfer composition in air that is capable of propagating a flame through a homogeneous mixture of the composition and air under the same test conditions.

As used herein, the term “Critical Pressure” refers to the pressure at or above which a fluid does not undergo a vapor-liquid phase transition no matter how much the temperature is varied.

### Chemicals, Abbreviations, and Acronyms

ACN: acetonitrile
ATL: atmospheric life time
2-BTP: 2-bromo-3,3,3-trifluoroprop-1-ene (CF₃CBr═CH₂)
CFO: chlorofluoroolefin
CFO-1316mxx: 2,3-dichloro-1,1,1,4,4,4-hexafluorobut-2-ene (CF₃CCl═CClCF₃)
CFO-1112: 1,2-dichloro-1,2-difluoroethylene (CFCl═CFCl)
CFC: chlorofluorocarbon
CFC-12: dichlorodifluoromethane (CF₂Cl₂)
CFC-11: trichlorofluoromethane (CFCl₃)
PFC: perfluorocarbon
PFC-C-318: octafluorocyclobutane (Cyclo-CF₂—CF₂—CF₂—CF₂—)
CFC-114: 1,2-dichloro-1,1,2,2-tetrafluoroethane (CF₂ClCF₂Cl)
CFC-113: 1,1,2-trichloro-1,2,2-trifluoroethane (CFCl₂CF₂Cl)
DME: dimethyl ether (CH₃OCH₃)
Methylal; DMM: dimethoxymethane (CH₃OCH₂OCH₃)
HFC: hydrofluorocarbon
HFC-32: difluoromethane (CH₂F₂)
HFC-152a: 1,1-difluoroethane (CHF₂CH₃)
HFC-134a: 1,1,1,2-tetrafluoroethane (CF₃CH₂F)
HFC-125: pentafluoroethane (CHF₂CF₃)
HFC-227ea: 1,1,1,2,3,3,3-heptafluoropropane (CF₃CHFCF₃)
HFC-245fa: 1,1,1,3,3-pentafluoropropane (CF₃CH₂CHF₂)
HFC-365mfc: 1,1,1,3,3-pentafluorobutane (CF₃CH₂CF₂CH₃)
HFE: hydrofluoroether
HFE-7100: 1,1,1,2,2,3,3,4,4-nonafluoro-4-methoxybutane (CF₃CF₂CF₂CF₂OCH₃)
HFC-4310mee: 1,1,1,2,2,3,4,5,5,5-decafluoropentane (CF₃CF₂CHFCHFCF₃)
HFO: hydrofluoroolefins
HFO-1336mzz: 1,1,1,4,4,4,-hexafluorobut-2-ene (CF₃CH═CHCF₃)
HFO-1224yd: CF₃CF═CHCl
HFO-1225ye: CF₃CF═CHF
HCFO: hydrochlorofluoroolefin
E-HCFO-1233zd: (E)-1-chloro-3,3,3-trifluoropropene (E-CF₃CH═CHCl)
Z-HCFO-1233zd: (Z)-1-chloro-3,3,3-trifluoropropene (Z-CF₃CH═CHCl)
E-HFO-1234ze: (E)-1,3,3,3-tetrafluoropropene (E-CF₃CH═CHF)
Z-HFO-1234ze: (Z)-1,3,3,3-tetrafluoropropene (Z-CF₃CH═CHF)
HFO-1234yf: 2,3,3,3-tetrafluoropropene (CF₃CF═CH₂)
F12E (HFO-1438mzz): (E)-1,1,1,4,4,5,5,5-octafluoropent-2-ene CF₃CH═CHC₂F₅
F33E: (E)-1,1,1,2,2,3,3,6,6,7,7,8,8,8-tetradecafluorooct-4-ene (E-C₃F₇CH═CHC₃F₇)
F24E: (E)-1,1,1,2,2,5,5,6,6,7,7,8,8,8-tetradecafluorooct-3-ene (E-C₂F₅CH═CHC₄F₉)
F44E: (E)-1,1,1,2,2,3,3,4,4,7,7,8,8,9,9,10,10,10-octadecafluorodec-5-ene (E-C₄F₉CH═CHC₄F₉)
HFIBO: hexafluoroisobutene oxide, 2,2-bis(trifluoromethyl)oxirane
NaOCl: sodium hypochlorite
PFP-2: perfluoropentene-2 (CF₃CF═CFCF₂CF₃)
PTC: phase transfer catalysis or phase transfer catalyst
NBP: normal boiling point
Tcr: critical temperature
COPh: coefficient of performance for heating

### IUPAC Names for Epoxides

Z-1234ze Epoxide: cis-2-fluoro-3-(trifluoromethyl)oxirane
E-1234ze Epoxide: trans-2-fluoro-3-(trifluoromethyl)oxirane
E-1336mzz Epoxide; E-F-11E oxide: trans-2,3-bis(trifluoromethyl)oxirane
Z-1336mzz Epoxide; E-F-11E oxide: cis-2,3-bis(trifluoromethyl)oxirane
F12E Epoxide: trans-2,3-bis(trifluoromethyl)oxirane
F13Ei Epoxide: trans-2-(trifluoromethyl)-3-(perfluoropropan-2-yl)oxirane
F33E Epoxide: trans-2,3-bis(perfluoropropyl)oxirane
F24E Epoxide: trans-2-(perfluorobutyl)-3-(perfluoroethyl)oxirane
F44E Epoxide: trans-2,3-bis(perfluorobutyl)oxirane
HFX-90 Epoxide: 2-(2,2,2-Trifluoroethoxy)-3-fluoro-2-(trifluoromethyl)-3-(perfluoroethyl)oxirane
1316mxx Epoxide: 2,3-dichloro-2,3-bis(trifluoromethyl)oxirane
E-1438ezy Epoxide: trans-2-fluoro-3-(perfluoropropan-2-yl)oxirane
Z-1438ezy Epoxide: cis-2-fluoro-3-(perfluoropropan-2-yl)oxirane
1,2-H-Hexafluorocyclopentene Epoxide: 2,2,3,3,4,4-hexafluoro-6-oxa-bicyclo[3.1.0]hexane
Perfluoroheptene-3 Epoxide: 2,3-difluoro-2-(perfluoroethyl)-3-(perfluoropropyl)oxirane
Perfluorooctene-2 Epoxide: 2,3-difluoro-2-(trifluoromethyl)-3-(perfluoropentyl)oxirane

### IUPAC Names for Olefins

Z-HCFO-1224yd: (Z)-1-chloro-2,3,3,3-tetrafluoropropene
E-HCFO-1224yd: (E)-1-chloro-2,3,3,3-tetrafluoropropene
Z-HCFO-1225ye: (Z)-1,2,3,3,3-pentafluoroprop-1-ene
E-HCFO-1225ye: (E)-1,2,3,3,3-pentafluoroprop-1-ene
E-HCFO-1233zd: E-1-chloro-3,3,3-trifluoropropene
Z-HCFO-1233zd: Z-1-chloro-3,3,3-trifluoropropene
E-HFO-1234ye: (E)-1,2,3,3-tetrafluoropropene
Z-HFO-1234ye: (Z)-1,2,3,3-tetrafluoropropene
E-HFO-1438mzz: (E)-1,1,1,4,4,5,5,5-octafluoropent-2-ene
Z-HFO-1438mzz: (Z)-1,1,1,4,4,5,5,5-octafluoropent-2-ene
Z-HFO-1234ze: (Z)-1,3,3,3-tetrafluoropropene
E-HFO-1234ze: (E)-1,3,3,3-tetrafluoropropene
E-HFO-1336mzz: (E)-1,1,1,4,4,4-hexafluorobut-2-ene
Z-HFO-1336mzz: (Z)-1,1,1,4,4,4-hexafluorobut-2-ene
F33E: (E)-1,1,1,2,2,3,3,6,6,7,7,8,8,8-tetradecafluorooct-4-ene
F24E: (E)-1,1,1,2,2,5,5,6,6,7,7,8,8,8-tetradecafluorooct-3-ene
F44E: (E)-1,1,1,2,2,3,3,4,4,7,7,8,8,9,9,10,10,10-octadecafluorodec-5-ene
HFX-90: 2-(2,2,2-trifluoroethoxy)-1,1,1,3,4,4,5,5,5-nonafluoropent-2-ene
CFO-1316mxx: (E/Z)-2,3-dichloro-1,1,1,4,4,4-hexafluorobut-2-ene
E-HFO-1438ezy: (E)-1,3,4,4,4-pentafluoro-3-(trifluoromethyl)but-1-ene
Z-HFO-1438ezy: (Z)-1,3,4,4,4-pentafluoro-3-(trifluoromethyl)but-1-ene
1,2-H-Hexafluorocyclopentene: 3,3,4,4,5,5-hexafluorocyclopent-1-ene
Perfluoropenetene-2: perfluoropent-2-ene
Perfluoroheptene-3: perfluorohept-3-ene
Perfluorooctene-2: perfluorooct-2-ene

### Compositions and Methods of Use

The fluoroepoxides described herein are useful for a variety of applications and compositions as detailed infra, including their use as aerosol propellants, refrigerants, solvents, cleaning agents, blowing agents (e.g., foam expansion agents) for thermoplastic and thermoset foams, heat transfer media, gaseous dielectrics, fire extinguishing, and suppression agents, power cycle working fluids, polymerization media, particulate removal fluids, carrier fluids, buffing abrasive agents, anesthetics, fumigants, sterilants, displacement drying agents, and gaseous dielectric materials.

In some embodiments, the fluoroepoxides have Formula (I):

[chemical structure]

wherein:

R¹ and R⁴ are each independently H, Cl, F, Br, I, a partially fluorinated C₁₋₄ alkoxy, or a perfluorinated C₁₋₄ alkoxy; and

R² is selected from H, Cl, F, Br, I, a partially fluorinated C₁₋₁₀ alkyl, a perfluorinated C₁₋₁₀ alkyl, a partially fluorinated C₁₋₄ alkoxy, and a perfluorinated C₁₋₄ alkoxy;

wherein at least one of R¹, R², and R⁴ is not H;
R³ is selected from partially fluorinated C₁₋₁₀ alkyl and perfluorinated C₁₋₁₀ alkyl;
   or alternatively, R² and R³ are each independently selected from partially fluorinated or perfluorinated C₁₋₅ alkylene, which together form a monocyclic ring.

In some embodiments, the compound of Formula (I) is the cis-isomer. In some embodiments, the compound of Formula (I) is the trans-isomer. In some embodiments, the compound of Formula I has the (Z)- or (E)-configuration and the composition is substantially free of the opposite stereoisomers.

Compounds labeled as trans herein have the (E) configuration, while compounds labeled as cis herein have the (Z) configuration.

For example, for a composition comprising a compound of Formula I having the (Z) configuration, the opposite stereoisomers would be the stereoisomers having the (E) configuration. In some embodiments, substantially free means less than 1% of the opposite stereoisomers. In some embodiments, substantially free means less than 0.5, 0.4, 0.3, 0.2 or 0.1% of the opposite stereoisomers.

In some embodiments, the compounds are non-cyclic. Accordingly, in some embodiments, R¹ and R⁴ are each independently H, Cl, F, a partially fluorinated C₁₋₄ alkoxy, or a perfluorinated C₁₋₄ alkoxy; and R² and R³ are each independently selected from partially fluorinated or perfluorinated C₁₋₁₀ alkyl.

In some embodiments of the non-cyclic compounds, R¹ and R⁴ are identical. In some embodiments of the non-cyclic compounds, R¹ and R⁴ are different. In some embodiments of the non-cyclic compounds, R¹ and R⁴ are each H. In some embodiments of the non-cyclic compounds, R¹ and R⁴ are each F. In some embodiments of the non-cyclic compounds, le and R⁴ are each Cl. In some embodiments of the non-cyclic compounds, le is a partially fluorinated C₁₋₄ alkoxy and R⁴ is H.

In some embodiments of the non-cyclic compounds, R² and R³ are identical. In some embodiments of the non-cyclic compounds, R² and R³ are different. In some embodiments, R² is H or F; and R³ is partially fluorinated or perfluorinated C₁₋₁₀ alkyl. In some embodiments of the non-cyclic compounds, R² and R³ are each independently selected from partially fluorinated or perfluorinated C₁₋₁₀ alkyl. In some embodiments of the non-cyclic compounds, R² and R³ are each an independently selected perfluorinated C₁₋₁₀ alkyl. In some embodiments of the non-cyclic compounds, R² and R³ are each an independently selected partially fluorinated C₁₋₁₀ alkyl. In some embodiments of the non-cyclic compounds, R² and R³ are each independently selected from partially fluorinated or perfluorinated C₁₋₆ alkyl. In some embodiments of the non-cyclic compounds, R² and R³ are each independently selected from partially fluorinated or perfluorinated C₁₋₆ alkyl. In some embodiments of the non-cyclic compounds, R² and R³ are each independently selected from partially fluorinated C₁₋₆ alkyl. In some embodiments of the non-cyclic compounds, R² and R³ are each independently selected from perfluorinated C₁₋₆ alkyl. In some embodiments of the non-cyclic compounds, R² and R³ are each independently CF₃, CF₂CF₃, CF(CF₃)₂, CF₂CF₂CF₃, CF₂CF₂CF₂CF₃, or CF₂CF₂CF₂CF₂CF₃.

In some embodiments:
R¹ and R⁴ are H;
R² is partially fluorinated or perfluorinated C₁₋₁₀ alkyl; and
R³ is partially fluorinated or perfluorinated C₁₋₁₀ alkyl.

In some embodiments:
R¹ and R⁴ are H;
R² is partially fluorinated or perfluorinated C₁₋₆ alkyl; and
R³ is partially fluorinated or perfluorinated C₁₋₆ alkyl.

In some embodiments:
R¹ and R⁴ are H;
R² is selected from CF₃, CF₂CF₃, CF(CF₃)₂, CF₂CF₂CF₃, CF₂CF₂CF₂CF₃, or CF₂CF₂CF₂CF₂CF₃,
R³ is selected from CF₃, CF₂CF₃, CF(CF₃)₂, CF₂CF₂CF₃, CF₂CF₂CF₂CF₃, or CF₂CF₂CF₂CF₂CF₃.

In some embodiments:
R¹ and R⁴ are each independently H, Cl, F, a partially fluorinated C₁₋₄ alkoxy, or a perfluorinated C₁₋₄ alkoxy;
R² is H, F, partially fluorinated C₁₋₁₀ alkyl, or perfluorinated C₁₋₁₀ alkyl;
wherein at least one of R¹, R², and R⁴ is not H; and
R³ is selected from partially fluorinated C₁₋₁₀ alkyl and perfluorinated C₁₋₁₀ alkyl.

In some embodiments:
R¹ and R⁴ are each independently H, Cl, F, or OCH₂CF₃;
R² is F, CF₃, CF₂CF₃, CF(CF₃)₂, CF₂CF₂CF₃, CF₂CF₂CF₂CF₃, or CF₂CF₂CF₂CF₂CF₃; and
R³ is CF₃, CF₂CF₃, CF(CF₃)₂, CF₂CF₂CF₃, CF₂CF₂CF₂CF₃, or CF₂CF₂CF₂CF₂CF₃.

In some embodiments:
R¹ and R⁴ are each independently H, Cl, or F;
R² is F, CF₃, CF₂CF₃, CF(CF₃)₂, CF₂CF₂CF₃, CF₂CF₂CF₂CF₃, or CF₂CF₂CF₂CF₂CF₃; and
R³ is CF₃, CF₂CF₃, CF(CF₃)₂, CF₂CF₂CF₃, CF₂CF₂CF₂CF₃, or CF₂CF₂CF₂CF₂CF₃.

In some embodiments, the compounds are cyclic. Accordingly, in some embodiments, le and R⁴ are each independently H, Cl, F, Br, I, a partially fluorinated C₁₋₄ alkoxy, or a perfluorinated C₁₋₄ alkoxy; and R² and R³ are each independently selected from partially fluorinated or perfluorinated C₁₋₅ alkylene, which together form a monocyclic ring.

In some embodiments of the cyclic compounds, R¹ and R⁴ are identical. In some embodiments of the cyclic compounds, R¹ and R⁴ are different. In some embodiments of the cycle compounds, R¹ and R⁴ are each independently H, Cl, F, a partially fluorinated C₁₋₄ alkoxy, or a perfluorinated C₁₋₄ alkoxy. In some embodiments of the cyclic compounds, le and R⁴ are each H. In some embodiments of the cyclic compounds, R¹ and R⁴ are each F. In some embodiments of the cyclic compounds, R¹ and R⁴ are each Cl. In some embodiments of the cyclic compounds, le is a partially fluorinated C₁₋₄ alkoxy and R⁴ is H.

In some embodiments, R² and R³ are each independently selected from partially fluorinated or perfluorinated C₁₋₅ alkylene, which together form a monocyclic ring. In some embodiments, R² and R³ are each independently selected from a perfluorinated C₁₋₅ alkylene, which together form a monocyclic ring. In some embodiments, R² and R³, together with the carbon atoms to which they are attached, form a 4-6 membered monocyclic ring. In some embodiments, R¹ and R⁴ are each independently H or F; and R² and R³ are each independently selected from partially fluorinated or perfluorinated C₁₋₅ alkylene, which together form a monocyclic ring. In some embodiments, R¹ and R⁴ are each independently H or F; and R² and R³ are each an independently selected perfluorinated C₁₋₂ alkylene, which together form a monocyclic ring.

In some embodiments:
R¹ and R⁴ are H;
R² is partially fluorinated or perfluorinated C₁₋₅ alkylene and
R³ is partially fluorinated or perfluorinated C₁₋₅ alkylene
wherein said R² and R³ are taken together form a monocyclic ring.

In some embodiments:
R¹ and R⁴ are H;
R² is partially fluorinated or perfluorinated C₁₋₂ alkylene; and
R³ is partially fluorinated or perfluorinated C₁₋₂ alkylene;
wherein said R² and R³ are taken together form a monocyclic ring.

In some embodiments:
R¹ and R⁴ are H;
R² is selected from —CF₂— and —CF₂CF₂—; and
R³ is selected from —CF₂— and —CF₂CF₂—;
wherein said R² and R³ are taken together form a 4-6 membered monocyclic ring.

In some embodiments, the compound of Formula (I) is selected from the group consisting of:
2,3-difluoro-2-(trifluoromethyl)oxirane;
2-fluoro-3-(trifluoromethyl)oxirane;
2,3-bis(trifluoromethyl)oxirane;
2-(trifluoromethyl)-3-(perfluoroethyl)oxirane;
2-(trifluoromethyl)-3-(perfluoropropan-2-yl)oxirane;
2,3-bis(perfluoropropyl)oxirane;
2-(perfluorobutyl)-3-(perfluoroethyl)oxirane;
2,3-bis(perfluorobutyl)oxirane;
2-(2,2,2-Trifluoroethoxy)-3-fluoro-2-(trifluoromethyl)-3-(perfluoroethyl)oxirane;
2,3-dichloro-2,3-bis(trifluoromethyl)oxirane;
2-fluoro-3-(perfluoropropan-2-yl)oxirane;
2,2,3,3,4,4-hexafluoro-6-oxa-bicyclo[3.1.0]hexane;
2,2,3,3-tetrafluoro-5-oxabicyclo[2.1.0]pentane;
2,3-difluoro-2-(perfluoroethyl)-3-(perfluoropropyl)oxirane; and
2,3-difluoro-2-(trifluoromethyl)-3-(perfluoropentyl)oxirane.

In some embodiments, the compound of Formula (I) is selected from the group consisting of:
(Z)-2,3-difluoro-2-(trifluoromethyl)oxirane;
(E)-2,3-difluoro-2-(trifluoromethyl)oxirane;
cis-2-fluoro-3-(trifluoromethyl)oxirane;
trans-2-fluoro-3-(trifluoromethyl)oxirane;
trans-2,3-bis(trifluoromethyl)oxirane;
cis-2,3-bis(trifluoromethyl)oxirane;
trans-2-(trifluoromethyl)-3-(perfluoroethyl)oxirane;
trans-2-(trifluoromethyl)-3-(perfluoropropan-2-yl)oxirane;
trans-2,3-bis(perfluoropropyl)oxirane;
trans-2-(perfluorobutyl)-3-(perfluoroethyl)oxirane;
trans-2,3-bis(perfluorobutyl)oxirane;
(Z)-2-(2,2,2-Trifluoroethoxy)-3-fluoro-2-(trifluoromethyl)-3-(perfluoroethyl)oxirane;
(E)-2-(2,2,2-Trifluoroethoxy)-3-fluoro-2-(trifluoromethyl)-3-(perfluoroethyl)oxirane;
cis-2,3-dichloro-2,3-bis(trifluoromethyl)oxirane;
trans-2,3-dichloro-2,3-bis(trifluoromethyl)oxirane;
trans-2-fluoro-3-(perfluoropropan-2-yl)oxirane;
cis-2-fluoro-3-(perfluoropropan-2-yl)oxirane;
cis-2,2,3,3,4,4-hexafluoro-6-oxa-bicyclo[3.1.0]hexane;
cis-2,2,3,3-tetrafluoro-5-oxabicyclo[2.1.0]pentane;
cis-2,3-difluoro-2-(perfluoroethyl)-3-(perfluoropropyl)oxirane;
trans-2,3-difluoro-2-(perfluoroethyl)-3-(perfluoropropyl)oxirane;
cis-2,3-difluoro-2-(trifluoromethyl)-3-(perfluoropentyl)oxirane; and
trans-2,3-difluoro-2-(trifluoromethyl)-3-(perfluoropentyl)oxirane.

In some embodiments, the component is one of the preceding compounds of Formula (I), wherein the compound is free of the opposite stereoisomers.

The compounds of Formula (I) provided herein include stereoisomers of the compounds. All stereoisomers, such as enantiomers and diastereomers, are intended unless otherwise indicated.

In some embodiments, the compound of Formula (I) is a compound of Formula (Ia), (Ib), (Ic), or (Id):

[chemical structure]

When the compound of Formula (I) described as the E or trans-isomer, it can be a mixture of compounds of Formula (Ia) and (Id). When the compound of Formula (I) described as the Z or cis-isomer, it can be a mixture of compounds of Formula (Ib) and (Ic).

The compounds of Formula (I) can be prepared by the procedure shown in the Examples—i.e., by oxidizing a halolefin using aqueous NaOCl, an organic solvent (e.g., toluene, xylene, or acetonitrile), and a phase transfer catalyst (e.g., a quaternary ammonium salt or quaternary phosphonium salt).

Any of the embodiments of the compounds described supra (or any combination of embodiments) can be used in the specific compositions (including the mixtures) or methods described infra.

In some embodiments, the compositions described in the sections below comprise at least one compound of Formula (I) (as defined in any of the preceding embodiments or selected from any one of the specific compounds of Formula (I)) and at least one hydrofluoroolefin (HFO), hydrochlorofluoroolefin (HCFO), hydrochlorofluorocarbon (HCFC), hydrofluorocarbon (HFC), hydrofluoroether (HFE), or hydrofluoroolefinic ether (HFOE).

### Refrigerants

The disclosed compounds and compositions can act as a working fluid used to carry heat from a heat source to a heat sink. Such heat transfer compositions may also be useful as a refrigerant in a cycle wherein the fluid undergoes a phase change; that is, from a liquid to a gas and back, or vice versa. Examples of heat transfer systems include but are not limited to air conditioners, freezers, refrigerators, heat pumps, water chillers, flooded evaporator chillers, direct expansion chillers, walk-in coolers, high temperature heat pumps, mobile refrigerators, mobile air conditioning units, immersion cooling systems, data-center cooling systems, and combinations thereof.

Mechanical vapor-compression refrigeration, air conditioning and heat pump systems include an evaporator, a compressor, a condenser, and an expansion device. A refrigeration cycle re-uses refrigerant in multiple steps producing a cooling effect in one step and a heating effect in a different step. The cycle can be described as follows: Liquid refrigerant enters an evaporator through an expansion device, and the liquid refrigerant boils in the evaporator, by withdrawing heat from the environment, at a low temperature to form a gas and produce cooling. Often air or a heat transfer fluid flows over or around the evaporator to transfer the cooling effect caused by the evaporation of the refrigerant in the evaporator to a body to be cooled. The low-pressure gas enters a compressor where the gas is compressed to raise its pressure and temperature. The higher-pressure (compressed) gaseous refrigerant then enters the condenser in which the refrigerant condenses and discharges its heat to the environment. The refrigerant returns to the expansion device through which the liquid expands from the higher-pressure level in the condenser to the low-pressure level in the evaporator, thus repeating the cycle.

A body to be cooled or heated may be defined as any space, location, object or body for which it is desirable to provide cooling or heating. Examples include spaces (open or enclosed) requiring air conditioning, cooling, or heating, such as a room, an apartment, or building, such as an apartment building, university dormitory, townhouse, or other attached house or single family home, hospitals, office buildings, supermarkets, college or university classrooms or administration buildings and automobile or truck passenger compartments. Additionally, a body to be cooled may include electronic devices, such as computer equipment, central processing units (cpu), data-centers, server banks, and personal computers among others.

By “in the vicinity of” is meant that the evaporator of the system containing the refrigerant composition is located either within or adjacent to the body to be cooled, such that air moving over the evaporator would move into or around the body to be cooled. In the process for producing heating, “in the vicinity of” means that the condenser of the system containing the refrigerant composition is located either within or adjacent to the body to be heated, such that the air moving over the evaporator would move into or around the body to be heated. In some embodiments, for heat transfer, “in the vicinity of” may mean that the body to be cooled is immersed directly in the heat transfer composition or tubes containing heat transfer compositions run into around internally, and out of electronic equipment, for instance.

Examples of refrigeration systems the disclosed compounds and compositions may be useful in are equipment including commercial, industrial or residential refrigerators and freezers, ice machines, self-contained coolers and freezers, flooded evaporator chillers, direct expansion chillers, walk-in and reach-in coolers and freezers, and combination systems. In some embodiments, the disclosed compounds and compositions may be used in supermarket refrigeration systems. Additionally, stationary applications may utilize a secondary loop system that uses a primary refrigerant to produce cooling in one location that is transferred to a remote location via a secondary heat transfer fluid.

In some embodiments, the compounds and compositions of the invention are useful in mobile heat transfer systems, including refrigeration, air conditioning, or heat pump systems or apparatus. In some embodiments, the compounds and compositions are useful in stationary heat transfer systems, including refrigeration, air conditioning, or heat pump systems or apparatus.

As used herein, mobile refrigeration, air conditioning, or heat pump systems refers to any refrigeration, air conditioner, or heat pump apparatus incorporated into a transportation unit for the road, rail, sea or air. Mobile air conditioning or heat pumps systems may be used in automobiles, trucks, railcars or other transportation systems. Mobile refrigeration may include transport refrigeration in trucks, airplanes, or rail cars. In addition, apparatus which are meant to provide refrigeration for a system independent of any moving carrier, known as “intermodal” systems, are including in the present inventions. Such intermodal systems include “containers” (combined sea/land transport) as well as “swap bodies” (combined road and rail transport).

As used herein, stationary air conditioning or heat pump systems are systems that are fixed in place during operation. A stationary air conditioning or heat pump system may be associated within or attached to buildings of any variety. These stationary applications may be stationary air conditioning and heat pumps, including but not limited to chillers, heat pumps, including residential and high temperature heat pumps, residential, commercial or industrial air conditioning systems, and including window, ductless, ducted, packaged terminal, and those exterior but connected to the building such as rooftop systems.

Stationary heat transfer may refer to systems for cooling electronic devices, such as immersion cooling systems, submersion cooling systems, phase change cooling systems, data-center cooling systems or simply liquid cooling systems.

In some embodiments, a method is provided for using the present compounds or compositions as a heat transfer fluid. The method comprises transporting said composition from a heat source to a heat sink.

In some embodiments, a method is provided for producing cooling comprising evaporating any of the present compounds or compositions in the vicinity of a body to be cooled, and thereafter condensing said composition.

In some embodiments, a method is provided for producing heating comprising condensing any of the present compounds or compositions in the vicinity of a body to be heated, and thereafter evaporating said compositions.

In some embodiments, the compound or composition is for use in heat transfer, wherein the working fluid is a heat transfer component. Preferably, the compound for use as a heat transfer component has a boiling point range of −60° C. to 300° C. In some embodiments, compound for use as a heat transfer component is selected from (Z)-2,3-difluoro-2-(trifluoromethyl)oxirane, (E)-2,3-difluoro-2-(trifluoromethyl)oxirane, cis-2-fluoro-3-(trifluoromethyl)oxirane, trans-2-fluoro-3-(trifluoromethyl)oxirane, trans-2,3-bis(trifluoromethyl)oxirane, cis-2,3-bis(trifluoromethyl)oxirane, trans-2-(trifluoromethyl)-3-(perfluoroethyl)oxirane, trans-2-(trifluoromethyl)-3-(perfluoropropan-2-yl)oxirane, trans-2,3-bis(perfluoropropyl)oxirane, trans-2-(perfluorobutyl)-3-(perfluoroethyl)oxirane, trans-2,3-bis(perfluorobutyl)oxirane, (Z)-2-(2,2,2-Trifluoroethoxy)-3-fluoro-2-(trifluoromethyl)-3-(perfluoroethyl)oxirane, (E)-2-(2,2,2-Trifluoroethoxy)-3-fluoro-2-(trifluoromethyl)-3-(perfluoroethyl)oxirane, cis-2,3-dichloro-2,3-bis(trifluoromethyl)oxirane, trans-2,3-dichloro-2,3-bis(trifluoromethyl)oxirane, trans-2-fluoro-3-(perfluoropropan-2-yl)oxirane, cis-2-fluoro-3-(perfluoropropan-2-yl)oxirane, cis-2,2,3,3,4,4-hexafluoro-6-oxa-bicyclo[3.1.0]hexane, cis-2,2,3,3-tetrafluoro-5-oxabicyclo[2.1.0]pentane, cis-2,3-difluoro-2-(perfluoroethyl)-3-(perfluoropropyl)oxirane, trans-2,3-difluoro-2-(perfluoroethyl)-3-(perfluoropropyl)oxirane, cis-2,3-difluoro-2-(trifluoromethyl)-3-(perfluoropentyl)oxirane, and trans-2,3-difluoro-2-(trifluoromethyl)-3-(perfluoropentyl)oxirane, or a mixture thereof. In some embodiments, the compound for use as a heat transfer component is (Z)-2,3-difluoro-2-(trifluoromethyl)oxirane, (E)-2,3-difluoro-2-(trifluoromethyl)oxirane, cis-2-fluoro-3-(trifluoromethyl)oxirane, trans-2-fluoro-3-(trifluoromethyl)oxirane, trans-2,3-bis(trifluoromethyl)oxirane, trans-2-(trifluoromethyl)-3-(perfluoroethyl)oxirane, trans-2-fluoro-3-(perfluoropropan-2-yl)oxirane, or a mixture thereof. In some embodiments, the component is one of the preceding compounds of Formula (I), wherein the compound is free of the opposite stereoisomers.

In some embodiments, the composition for use in heat transfer further comprises difluoromethane, 1,1-difluoroethane, 1,1,1,2-tetrafluoroethane, pentafluoroethane, 1,1,1,2,3,3,3-heptafluoropropane, (E)-1,3,3,3-tetrafluoroprop-1-ene (E-HFO-1234ze), 2,3,3,3-tetrafluoroprop-1-ene (HFO-1234yf), (E)-1,1,1,4,4,4-hexafluorobut-2-ene (E-HFO-1336mzz), (Z)-1,1,1,4,4,4-hexafluorobut-2-ene (Z-1336mzz), (E)-1-chloro-3,3,3-trifluoropropene (E-1233zd), (Z)-1-chloro-3,3,3-trifluoropropene (Z-1233zd), (Z)-1-chloro-2,3,3,3-tetrafluoropropene (Z-HCFO-1224yd), (E)-1-chloro-2,3,3,3-tetrafluoropropene (E-HCFO-1224yd), (Z)-1,3,3,3-tetrafluoroprop-1-ene (Z-HFO-1234ze), (E)-1,2,3,3-tetrafluoropropene (E-HFO-1234ye), (Z)-1,2,3,3-tetrafluoropropene (Z-HFO-1234ye), (E)-1,1,1,4,4,5,5,5-octafluoro-2-pentene (E-HFO-1438mzz), (Z)-1,1,1,4,4,5,5,5-octafluoro-2-pentene (Z-HFO-1438mzz), (E)-1,3,4,4,4-pentafluoro-3-(trifluoromethyl)but-1-ene (E-HFO-1438ezy), (Z)-1,3,4,4,4-pentafluoro-3-(trifluoromethyl)but-1-ene (Z-HFO-1438ezy), 1,1,1,3,3-pentafluoropropane (HFC-245fa), 1-methoxyheptafluoropropane (HFE-7000), 1,1,1,3,3-pentafluorobutane (HFC-365mfc), 1,1,1,2,2,3,4,5,5,5-decafluoropentane (HFC-4310mee), 1,1,1,2,2,3,3,4,4-nonafluoro-4-methoxybutane (HFE-7100), methyl perfluoroheptene ether isomers (found as a mixture in Vertrel® HFX-11), or (2E)-1,1,1,4,5,5,5-heptafluoro-4-(trifluoromethyl)pent-2-ene (HFO-153-10mzzy), or a mixture thereof.

In some embodiments, the composition for use in heating or heat transfer comprises about 0.1% to 100%, about 0.1% to about 99%, about 1% to about 99%, about 10% to 99%, about 20% to about 99%, about 30% to about 90%, about 40% to about 99%, about 50% to about 99%, about 60% to about 99%, about 70% to about 99%, about 80% to about 99%, about 90% to about 99%, about 40% to about 90%, about 50% to about 90%, about 60% to about 90%, about 70% to about 90%, about 60% to about 80%, or about 50% to about 70% w/w of the compound of Formula (I) or a mixture of compounds of Formula (I). In some embodiments, the composition consists of the compound of Formula (I), or a mixture of compound of Formula (I).

In some embodiments, the compounds and composition of the invention are for use in refrigeration or air conditioning. Preferably, the compound for use as refrigerant or air conditioning component has a boiling point range of −80° C. to 35° C. For some chiller applications, the boiling point range is preferably also use 0° C. to 35° C.

In some embodiments, the compound (i.e., the refrigerant or air conditioning component) is selected from (Z)-2,3-difluoro-2-(trifluoromethyl)oxirane, (E)-2,3-difluoro-2-(trifluoromethyl)oxirane, trans-2-fluoro-3-(trifluoromethyl)oxirane, trans-2,3-bis(trifluoromethyl)oxirane, or a mixture thereof. In some embodiments, the component is one of the preceding compounds of Formula (I), wherein the compound is free of the opposite stereoisomers.

In some embodiments, the refrigerant or air conditioning composition further comprises difluoromethane (HFC-32), 1,1-difluoroethane (HFC-152a), 1,1,1,2-tetrafluoroethane (HFC-134a), 1,1,2,2-tetrafluoroethane (HFC-134), pentafluoroethane (HFC-125), 1,1,1-trifluoroethane (HFC-143a), 1,1,1,2,3,3,3-heptafluoropropane, (E)-1,3,3,3-tetrafluoroprop-1-ene (E-HFO-1234ze), 2,3,3,3-tetrafluoroprop-1-ene (HFO-1234yf), (E)-1,1,1,4,4,4-hexafluorobut-2-ene (E-1336mzz), (Z)-1,1,1,4,4,4-hexafluorobut-2-ene (Z-1336mzz), (E)-1-chloro-3,3,3-trifluoropropene (E-1233zd), (Z)-1-chloro-3,3,3-trifluoropropene (Z-1233zd), (Z)-1-chloro-2,3,3,3-tetrafluoropropene (Z-HCFO-1224yd), (E)-1-chloro-2,3,3,3-tetrafluoropropene (E-HCFO-1224yd), 3,3,3-trifluoropropene (HFO-1243zf), (Z)-1,3,3,3-tetrafluoroprop-1-ene (Z-HFO-1234ze), (E)-1,2,3,3-tetrafluoropropene (E-HFO-1234ye), (Z)-1,2,3,3-tetrafluoropropene (Z-HFO-1234ye), (E)-1,1,1,4,4,5,5,5-octafluoro-2-pentene (E-HFO-1438mzz), (Z)-1,1,1,4,4,5,5,5-octafluoro-2-pentene (Z-HFO-1438mzz), (E)-1,3,4,4,4-pentafluoro-3-(trifluoromethyl)but-1-ene (E-HFO-1438ezy), (Z)-1,3,4,4,4-pentafluoro-3-(trifluoromethyl)but-1-ene (Z-HFO-1438ezy), 1,1,1,3,3-pentafluoropropane (HFC-245fa), 1-methoxyheptafluoropropane (HFE-7000), 1,1,1,3,3-pentafluorobutane (HFC-365mfc), 1,1,1,2,2,3,3,4,4-nonafluoro-4-methoxybutane (HFE-7100), trans-1,2-dichloroethylene, 2-bromo-1,1,1-trifluoro-2-propene, E-1,2-dichloro-1,2-difluoroethylene, Z-1,2-dichloro-1,2-difluoroethylene, perfluoroethyl perfluoroisopropyl ketone (F-ethyl isopropyl ketone), E-HFO-1,2,3,3,3-pentafluoropropene (E-HFO-1225ye), Z-HFO-1,2,3,3,3-pentafluoropropene (Z-HFO-1225ye), CF₃I, carbon dioxide, nitrogen, and argon.

In some embodiments, the composition comprises about 0.1% to 100%, about 0.1% to about 99%, about 1% to about 99%, about 10% to 99%, about 20% to about 99%, about 10% to about 99%, about 30% to about 99%, about 40% to about 99%, about 50% to about 99%, about 60% to about 99%, about 70% to about 99%, about 80% to about 99%, about 90% to about 99%, about 40% to about 90%, about 50% to about 90%, about 60% to about 90%, about 70% to about 90%, about 60% to about 80%, or about 50% to about 70% w/w of the compound of Formula (I) or a mixture of compounds of Formula (I). In some embodiments, the composition consists of the compound of Formula (I), or a mixture of compound of Formula (I).

In some embodiments, compounds of the present invention may be useful for reducing or eliminating the flammability of flammable refrigerants. Compositions of present invention may further comprise the following flammable refrigerants: difluoromethane, 1,1-difluoroethane, (Z)-1,3,3,3-tetrafluoroprop-1-ene (Z-HFO-1234ze), (E)-1,3,3,3-tetrafluoroprop-1-ene (E-HFO-1234ze), 2,3,3,3-tetrafluoroprop-1-ene, (E)-1,2,3,3-tetrafluoropropene (E-HFO-1234ye), and (Z)-1,2,3,3-tetrafluoropropene (Z-HFO-1234ye).

In some embodiments, provided herein is a method for reducing the flammability of a flammable refrigerant comprising adding a composition comprising a fluorinated epoxide as disclosed herein to a flammable refrigerant.

The compounds or compositions disclosed herein may be useful as a replacement for a currently used (“incumbent”) refrigerant, including but not limited to R-123 (or HFC-123, 2,2-dichloro-1,1,1-trifluoroethane), R-11 (or CFC-11, trichlorofluoromethane), R-12 (or CFC-12, dichlorodifluoromethane), HFC-134a (1,1,1,2-tetrafluoroethane), HFC-32 (difluoromethane), R-22 (chlorodifluoromethane), R-245fa (or HFC-245fa, 1,1,1,3,3-pentafluoropropane), R-114 (or CFC-114, 1,2-dichloro-1,1,2,2-tetrafluoroethane), R-236fa (or HFC-236fa, 1,1,1,3,3,3-hexafluoropropane), R-236ea (or HFC-236ea, 1,1,1,2,3,3-hexafluoropropane), R-124 (or HCFC-124, 2-chloro-1,1,1,2-tetrafluoroethane), among others.

As used herein, the term “incumbent refrigerant” shall be understood to mean the refrigerant for which the heat transfer system was designed to operate, or the refrigerant that is resident in the heat transfer system.

Often replacement refrigerants are most useful if capable of being used in the original refrigeration equipment designed for a different refrigerant, e.g., with minimal to no system modifications. In many applications, some embodiments of the disclosed compositions are useful as refrigerants and provide at least comparable cooling performance (meaning cooling capacity) as the refrigerant for which a replacement is being sought.

In some embodiments is provided a method for operating a heat transfer system or for transferring heat that is designed to operate with an incumbent refrigerant by charging an empty system with a compound or composition of the present invention, or by substantially replacing said incumbent refrigerant with a compound or composition of the present invention.

As used herein, the term “substantially replacing” shall be understood to mean allowing the incumbent refrigerant to drain from the system, or pumping the incumbent refrigerant from the system, and then charging the system with a compound or composition of the present invention. The system may be flushed with one or more quantities of the replacement refrigerant before being charged. It shall be understood that in some embodiments, some small quantity of the incumbent refrigerant may be present in the system after the system has been charged with the compound or composition of the present invention.

In another embodiment is provided a method for recharging a heat transfer system that contains an incumbent refrigerant and a lubricant, said method comprising substantially removing the incumbent refrigerant from the heat transfer system while retaining a substantial portion of the lubricant in said system and introducing one of the present compounds or compositions to the heat transfer system. In some embodiments, the lubricant in the system is partially replaced.

In some embodiments, the compounds and compositions of the present invention may be used to top-off a refrigerant charge in a chiller. For example, if a chiller using HCFC-123 has diminished performance due to leakage of refrigerant, the compounds or compositions as disclosed herein may be added to bring performance back up to specification.

In some embodiments, a heat exchange system containing any the presently disclosed compounds or compositions is provided, wherein said system is selected from the group consisting of air conditioners, freezers, refrigerators, heat pumps, water chillers, flooded evaporator chillers, direct expansion chillers, walk-in coolers, heat pumps, mobile refrigerators, mobile air conditioning units, and systems having combinations thereof.

Additionally, the compounds or compositions provided herein may be useful in secondary loop systems wherein these compositions serve as the primary refrigerant thus providing cooling to a secondary heat transfer fluid that thereby cools a remote location.

The compounds and compositions of the present invention may have some temperature glide in the heat exchangers. Thus, the systems may operate more efficiently if the heat exchangers are operated in counter-current mode or cross-current mode with counter-current tendency. Counter-current tendency means that the closer the heat exchanger can get to counter-current mode the more efficient the heat transfer. Thus, air conditioning heat exchangers, in particular evaporators, are designed to provide some aspect of counter-current tendency.

Therefore, provided herein is an air conditioning or heat pump system wherein said system includes one or more heat exchangers (either evaporators, condensers or both) that operate in counter-current mode or cross-current mode with counter-current tendency.

In some embodiments, provided herein is a refrigeration system wherein said system includes one or more heat exchangers (either evaporators, condensers or both) that operate in counter-current mode or cross-current mode with counter-current tendency.

In some embodiments, the refrigeration, air conditioning or heat pump system is a stationary refrigeration, air conditioning or heat pump system. In some embodiments the refrigeration, air conditioning, or heat pump system is a mobile refrigeration, air conditioning or heat pump system.

Additionally, in some embodiments, the disclosed compounds or compositions may function as primary refrigerants in secondary loop systems that provide cooling to remote locations by use of a secondary heat transfer fluid, which may comprise water, an aqueous salt solution (e.g., calcium chloride), a glycol, carbon dioxide, or a fluorinated hydrocarbon fluid (meaning an HFC, HCFC, HFO, HCFO, CFO, or PFC). In this case, the secondary heat transfer fluid is the body to be cooled as it is adjacent to the evaporator and is cooled before moving to a second remote body to be cooled. In other embodiments, the disclosed compounds or compositions may function as the secondary heat transfer fluid, thus transferring or providing cooling (or heating) to the remote location).

In some embodiments, the compositions provided herein comprise one or more non-refrigerant components (also referred to herein as additives) selected from the group consisting of lubricants, dyes (including UV dyes), solubilizing agents, compatibilizers, stabilizers, tracers, perfluoropolyethers, anti-wear agents, extreme pressure agents, corrosion and oxidation inhibitors, metal surface energy reducers, metal surface deactivators, free radical scavengers, foam control agents, viscosity index improvers, pour point depressants, detergents, viscosity adjusters, and mixtures thereof. Indeed, many of these optional non-refrigerant components fit into one or more of these categories and may have qualities that lend themselves to achieve one or more performance characteristic.

In some embodiments, one or more non-refrigerant components are present in small amounts relative to the overall composition. In some embodiments, the amount of additive(s) concentration in the disclosed compositions is from less than about 0.1 weight percent to as much as about 5 weight percent of the total composition. In some embodiments of the present invention, the additives are present in the disclosed compositions in an amount between about 0.1 weight percent to about 5 weight percent of the total composition or in an amount between about 0.1 weight percent to about 3.5 weight percent. The additive component(s) selected for the disclosed composition is selected on the basis of the utility and/or individual equipment components or the system requirements.

In one embodiment, the lubricant is selected from the group consisting of mineral oil, alkylbenzene, polyol esters, polyalkylene glycols, polyvinyl ethers, polycarbonates, perfluoropolyethers, silicones, silicate esters, phosphate esters, paraffins, naphthenes, polyalpha-olefins, and combinations thereof.

The lubricants as disclosed herein may be commercially available lubricants. For instance, the lubricant may be paraffinic mineral oil, sold by BVA Oils as BVM 100 N, naphthenic mineral oils sold by Crompton Co. under the trademarks Suniso® 1GS, Suniso® 3GS and Suniso® SGS, naphthenic mineral oil sold by Pennzoil under the trademark Sontex® 372LT, naphthenic mineral oil sold by Calumet Lubricants under the trademark Calumet® RO-30, linear alkylbenzenes sold by Shrieve Chemicals under the trademarks Zerol® 75, Zerol® 150 and Zerol® 500 and branched alkylbenzene sold by Nippon Oil as HAB 22, polyol esters (POEs) sold under the trademark Castrol® 100 by Castrol, United Kingdom, polyalkylene glycols (PAGs) such as RL-488A from Dow (Dow Chemical, Midland, Mich.), and mixtures thereof, meaning mixtures of any of the lubricants disclosed in this paragraph.

Notwithstanding the above weight ratios for compositions disclosed herein, it is understood that in some heat transfer systems, while the composition is being used, it may acquire additional lubricant from one or more equipment components of such heat transfer system. For example, in some refrigeration, air conditioning and heat pump systems, lubricants may be charged in the compressor and/or the compressor lubricant sump. Such lubricant would be in addition to any lubricant additive present in the refrigerant in such a system. In use, the refrigerant composition when in the compressor may pick up an amount of the equipment lubricant to change the refrigerant-lubricant composition from the starting ratio.

The non-refrigerant component used with the compositions of the present invention may include at least one dye. The dye may be at least one ultra-violet (UV) dye. As used herein, “ultra-violet” dye is defined as a UV fluorescent or phosphorescent composition that absorbs light in the ultra-violet or “near” ultra-violet region of the electromagnetic spectrum. The fluorescence produced by the UV fluorescent dye under illumination by a UV light that emits at least some radiation with a wavelength in the range of from 10 nanometers to about 775 nanometers may be detected.

UV dye is a useful component for detecting leaks of the composition by permitting one to observe the fluorescence of the dye at or in the vicinity of a leak point in an apparatus (e.g., refrigeration unit, air-conditioner or heat pump). The UV emission, e.g., fluorescence from the dye may be observed under an ultra-violet light. Therefore, if a composition containing such a UV dye is leaking from a given point in an apparatus, the fluorescence can be detected at the leak point, or in the vicinity of the leak point.

In some embodiments, the UV dye may be a fluorescent dye. In some embodiments, the fluorescent dye is selected from the group consisting of naphthalimides, perylenes, coumarins, anthracenes, phenanthracenes, xanthenes, thioxanthenes, naphthoxanthenes, fluoresceins, and derivatives of said dye, and combinations thereof, meaning mixtures of any of the foregoing dyes or their derivatives disclosed in this paragraph.

Another non-refrigerant component which may be used with the compositions of the present invention may include at least one solubilizing agent selected to improve the solubility of one or more dye in the disclosed compositions. In some embodiments, the weight ratio of dye to solubilizing agent ranges from about 99:1 to about 1:1. The solubilizing agents include at least one compound selected from the group consisting of hydrocarbons, hydrocarbon ethers, polyoxyalkylene glycol ethers (such as dipropylene glycol dimethyl ether), amides, nitriles, ketones, chlorocarbons (such as methylene chloride, trichloroethylene, chloroform, or mixtures thereof), esters, lactones, aromatic ethers, fluoroethers, and 1,1,1-trifluoroalkanes and mixtures thereof, meaning mixtures of any of the solubilizing agents disclosed in this paragraph.

In some embodiments, the non-refrigerant component comprises at least one compatibilizer to improve the compatibility of one or more lubricants with the disclosed compositions. The compatibilizer may be selected from the group consisting of hydrocarbons, hydrocarbon ethers, polyoxyalkylene glycol ethers (such as dipropylene glycol dimethyl ether), amides, nitriles, ketones, chlorocarbons (such as methylene chloride, trichloroethylene, chloroform, or mixtures thereof), esters, lactones, aromatic ethers, fluoroethers, 1,1,1-trifluoroalkanes, and mixtures thereof, meaning mixtures of any of the compatibilizers disclosed in this paragraph.

The solubilizing agent and/or compatibilizer may be selected from the group consisting of hydrocarbon ethers consisting of the ethers containing only carbon, hydrogen and oxygen, such as dimethyl ether (DME) and mixtures thereof, meaning mixtures of any of the hydrocarbon ethers disclosed in this paragraph.

The compatibilizer may be linear or cyclic aliphatic or aromatic hydrocarbon compatibilizer containing from 3 to 15 carbon atoms. The compatibilizer may be at least one hydrocarbon, which may be selected from the group consisting of at least propanes, including propylene and propane, butanes, including n-butane and isobutene, pentanes, including n-pentane, isopentane, neopentane and cyclopentane, hexanes, octanes, nonane, and decanes, among others. Commercially available hydrocarbon compatibilizers include but are not limited to those from Exxon Chemical (USA) sold under the trademarks Isopar® H, a mixture of undecane (C₁₁) and dodecane (C₁₂) (a high purity C₁₁ to C₁₂ iso-paraffinic), Aromatic 150 (a C₉ to C₁₁ aromatic) (Aromatic 200 (a C₉ to C₁₅ aromatic) and Naptha 140 (a mixture of C₅ to C₁₁ paraffins, naphthenes and aromatic hydrocarbons) and mixtures thereof, meaning mixtures of any of the hydrocarbons disclosed in this paragraph.

The compatibilizer may alternatively be at least one polymeric compatibilizer. The polymeric compatibilizer may be a random copolymer of fluorinated and non-fluorinated acrylates, wherein the polymer comprises repeating units of at least one monomer represented by the formulae CH₂═C(R¹)CO₂R², CH₂═C(R³)C₆H₄R⁴, and CH₂═C(R⁵)C₆H₄XR⁶, wherein X is oxygen or sulfur; R¹, R³, and R⁵ are independently selected from the group consisting of H and C₁-C₄ alkyl radicals; and R², R⁴, and R⁶ are independently selected from the group consisting of carbon-chain-based radicals containing C, and F, and may further contain H, Cl, ether oxygen, or sulfur in the form of thioether, sulfoxide, or sulfone groups and mixtures thereof. Examples of such polymeric compatibilizers include those commercially available from E. I. du Pont de Nemours and Company, (Wilmington, Del., 19898, USA) under the trademark Zonyl® PHS. Zonyl® PHS is a random copolymer made by polymerizing 40 weight percent CH₂═C(CH₃)CO₂CH₂CH₂(CF₂CF₂)ₘF (also referred to as Zonyl® fluoromethacrylate or ZFM) wherein m is from 1 to 12, primarily 2 to 8, and 60 weight percent lauryl methacrylate (CH₂═C(CH₃)CO₂(CH₂)₁₁CH₃, also referred to as LMA).

In some embodiments, the compatibilizer component contains from about 0.01 to 30 weight percent (based on total amount of compatibilizer) of an additive which reduces the surface energy of metallic copper, aluminum, steel, or other metals and metal alloys thereof found in heat exchangers in a way that reduces the adhesion of lubricants to the metal. Examples of metal surface energy reducing additives include those commercially available from DuPont under the trademarks Zonyl® FSA, Zonyl® FSP, and Zonyl® FSJ.

Another non-refrigerant component which may be used with the compositions of the present invention may be a metal surface deactivator. The metal surface deactivator is selected from the group consisting of areoxalyl bis (benzylidene) hydrazide (CAS reg no. 6629-10-3), N,N′-bis(3,5-di-tert-butyl-4-hydroxyhydrocinnamoylhydrazine (CAS reg no. 32687-78-8), 2,2,′-oxamidobis-ethyl-(3,5-di-tert-butyl-4-hydroxyhydrocinnamate (CAS reg no. 70331-94-1), N,N′-(disalicyclidene)-1,2-diaminopropane (CAS reg no. 94-91-7) and ethylenediaminetetra-acetic acid (CAS reg no. 60-00-4) and its salts, and mixtures thereof, meaning mixtures of any of the metal surface deactivators disclosed in this paragraph.

The non-refrigerant component used with the compositions of the present invention may alternatively be a stabilizer selected from the group consisting of hindered phenols, thiophosphates, butylated triphenylphosphorothionates, organo phosphates, or phosphites, aryl alkyl ethers, terpenes, terpenoids, epoxides, fluorinated epoxides, oxetanes, ascorbic acid, thiols, lactones, thioethers, amines, nitromethane, alkylsilanes, benzophenone derivatives, aryl sulfides, divinyl terephthalic acid, diphenyl terephthalic acid, hydrazones, such as acetaldehyde dimethylhydrazone, ionic liquids, and mixtures thereof, meaning mixtures of any of the stabilizers disclosed in this paragraph.

The stabilizer may be selected from the group consisting of tocopherol; hydroquinone; t-butyl hydroquinone; monothiophosphates; and dithiophosphates, commercially available from Ciba Specialty Chemicals, Basel, Switzerland, hereinafter “Ciba”, under the trademark Irgalube® 63; dialkylthiophosphate esters, commercially available from Ciba under the trademarks Irgalube® 353 and Irgalube® 350, respectively; butylated triphenylphosphorothionates, commercially available from Ciba under the trademark Irgalube® 232; amine phosphates, commercially available from Ciba under the trademark Irgalube® 349 (Ciba); hindered phosphites, commercially available from Ciba as Irgafos® 168 and Tris-(di-tert-butylphenyl)phosphite, commercially available from Ciba under the trademark Irgafos® OPH; (Di-n-octyl phosphite); and iso-decyl diphenyl phosphite, commercially available from Ciba under the trademark Irgafos® DDPP; trialkyl phosphates, such as trimethyl phosphate, triethylphosphate, tributyl phosphate, trioctyl phosphate, and tri(2-ethylhexyl)phosphate; triaryl phosphates including triphenyl phosphate, tricresyl phosphate, and trixylenyl phosphate; and mixed alkyl-aryl phosphates including isopropylphenyl phosphate (IPPP), and bis(t-butylphenyl)phenyl phosphate (TBPP); butylated triphenyl phosphates, such as those commercially available under the trademark Syn-O-Ad® including Syn-O-Ad® 8784; tert-butylated triphenyl phosphates such as those commercially available under the trademark Durad® 620; isopropylated triphenyl phosphates such as those commercially available under the trademarks Durad® 220 and Durad® 110; anisole; 1,4-dimethoxybenzene; 1,4-diethoxybenzene; 1,3,5-trimethoxybenzene; myrcene, alloocimene, limonene (in particular, d-limonene); retinal; pinene; menthol; geraniol; farnesol; phytol; Vitamin A; terpinene; delta-3-carene; terpinolene; phellandrene; fenchene; dipentene; caratenoids, such as lycopene, beta carotene, and xanthophylls, such as zeaxanthin; retinoids, such as hepaxanthin and isotretinoin; bornane; 1,2-propylene oxide; 1,2-butylene oxide; n-butyl glycidyl ether; trifluoromethyloxirane; 1,1-bis(trifluoromethyl)oxirane; 3-ethyl-3-hydroxymethyl-oxetane, such as OXT-101 (Toagosei Co., Ltd); 3-ethyl-3-((phenoxy)methyl)-oxetane, such as OXT-211 (Toagosei Co., Ltd); 3-ethyl-3-((2-ethyl-hexyloxy)methyl)-oxetane, such as OXT-212 (Toagosei Co., Ltd); ascorbic acid; methanethiol (methyl mercaptan); ethanethiol (ethyl mercaptan); Coenzyme A; dimercaptosuccinic acid (DMSA); grapefruit mercaptan ((R)-2-(4-methylcyclohex-3-enyl)propane-2-thiol)); cysteine ((R)-2-amino-3-sulfanyl-propanoic acid); lipoamide (1,2-dithiolane-3-pentanamide); 5,7-bis(1,1-dimethylethyl)-3-[2,3(or 3,4)-dimethylphenyl]-2(3H)-benzofuranone, commercially available from Ciba under the trademark Irganox® HP-136; benzyl phenyl sulfide; diphenyl sulfide; diisopropylamine; dioctadecyl 3,3′-thiodipropionate, commercially available from Ciba under the trademark Irganox® PS 802 (Ciba); didodecyl 3,3′-thiopropionate, commercially available from Ciba under the trademark Irganox® PS 800; di-(2,2,6,6-tetramethyl-4-piperidyl)sebacate, commercially available from Ciba under the trademark Tinuvin® 770; poly-(N-hydroxyethyl-2,2,6,6-tetramethyl-4-hydroxy-piperidyl succinate, commercially available from Ciba under the trademark Tinuvin® 622LD (Ciba); methyl bis tallow amine; bis tallow amine; phenol-alpha-naphthylamine; bis(dimethylamino)methylsilane (DMAMS); tris(trimethylsilyl)silane (TTMSS); vinyltriethoxysilane; vinyltrimethoxysilane; 2,5-difluorobenzophenone; 2′,5′-dihydroxyacetophenone; 2-aminobenzophenone; 2-chlorobenzophenone; benzyl phenyl sulfide; diphenyl sulfide; dibenzyl sulfide; ionic liquids; and mixtures and combinations thereof.

The additive used with the compositions of the present invention may alternatively be an ionic liquid stabilizer. The ionic liquid stabilizer may be selected from the group consisting of organic salts that are liquid at room temperature (approximately 25° C.), those salts containing cations selected from the group consisting of pyridinium, pyridazinium, pyrimidinium, pyrazinium, imidazolium, pyrazolium, thiazolium, oxazolium and triazolium and mixtures thereof; and anions selected from the group consisting of [BF₄]⁻, [PF₆]⁻, [SbF₆]⁻, [CF₃SO₃]⁻, [HCF₂CF₂SO₃]⁻, [CF₃HFCCF₂SO₃]⁻, [HCClFCF₂SO₃]⁻, [(CF₃SO₂)₂N]⁻, [(CF₃CF₂SO₂)₂N]⁻, [(CF₃SO₂)₃C]⁻, [CF₃CO₂]⁻, and F⁻, and mixtures thereof. In some embodiments, ionic liquid stabilizers are selected from the group consisting of emim BF₄ (1-ethyl-3-methylimidazolium tetrafluoroborate); bmim BF₄ (1-butyl-3-methylimidazolium tetraborate); emim PF₆ (1-ethyl-3-methylimidazolium hexafluorophosphate); and bmim PF₆ (1-butyl-3-methylimidazolium hexafluorophosphate), all of which are available from Fluka (Sigma-Aldrich).

In some embodiments, the stabilizer may be a hindered phenol, which is any substituted phenol compound, including phenols comprising one or more substituted or cyclic, straight chain, or branched aliphatic substituent group, such as, alkylated monophenols including 2,6-di-tert-butyl-4-methylphenol; 2,6-di-tert-butyl-4-ethylphenol; 2,4-dimethyl-6-tertbutylphenol; tocopherol; and the like, hydroquinone and alkylated hydroquinones including t-butyl hydroquinone, other derivatives of hydroquinone; and the like, hydroxylated thiodiphenyl ethers, including 4,4′-thio-bis(2-methyl-6-tert-butylphenol); 4,4′-thiobis(3-methyl-6-tertbutylphenol); 2,2′-thiobis(4methyl-6-tert-butylphenol); and the like, alkylidene-bisphenols including: 4,4′-methylenebis(2,6-di-tert-butylphenol); 4,4′-bis(2,6-di-tert-butylphenol); derivatives of 2,2′- or 4,4-biphenoldiols; 2,2′-methylenebis(4-ethyl-6-tertbutylphenol); 2,2′-methylenebis(4-methyl-6-tertbutylphenol); 4,4-butylidenebis(3-methyl-6-tert-butylphenol); 4,4-isopropylidenebis(2,6-di-tert-butylphenol); 2,2′-methylenebis(4-methyl-6-nonylphenol); 2,2′-isobutylidenebis(4,6-dimethylphenol; 2,2′-methylenebis(4-methyl-6-cyclohexylphenol, 2,2- or 4,4-biphenyldiols including 2,2′-methylenebis(4-ethyl-6-tert-butylphenol); butylated hydroxytoluene (BHT, or 2,6-di-tert-butyl-4-methylphenol), bisphenols comprising heteroatoms including 2,6-di-tert-alpha-dimethylamino-p-cresol, 4,4-thiobis(6-tert-butyl-m-cresol); and the like; acylaminophenols; 2,6-di-tert-butyl-4(N,N′-dimethylaminomethylphenol); sulfides including; bis(3-methyl-4-hydroxy-5-tert-butylbenzyl)sulfide; bis(3,5-di-tert-butyl-4-hydroxybenzyl)sulfide and mixtures thereof, meaning mixtures of any of the phenols disclosed in this paragraph.

The non-refrigerant component which is used with compositions of the present invention may alternatively be a tracer. The tracer may be two or more tracer compounds from the same class of compounds or from different classes of compounds. In some embodiments, the tracer is present in the compositions at a total concentration of about 50 parts per million by weight (ppm) to about 1000 ppm, based on the weight of the total composition. In other embodiments, the tracer is present at a total concentration of about 50 ppm to about 500 ppm. Alternatively, the tracer is present at a total concentration of about 100 ppm to about 300 ppm.

The tracer may be selected from the group consisting of hydrofluorocarbons (HFCs), deuterated hydrofluorocarbons, perfluorocarbons, fluoroethers, brominated compounds, iodated compounds, alcohols, aldehydes and ketones, nitrous oxide and combinations thereof. Alternatively, the tracer may be selected from the group consisting of fluoroethane, 1,1,-difluoroethane, 1,1,1-trifluoroethane, 1,1,1,3,3,3-hexafluoropropane, 1,1,1,2,3,3,3-heptafluoropropane, 1,1,1,3,3-pentafluoropropane, 1,1,1,3,3-pentafluorobutane, 1,1,1,2,3,4,4,5,5,5-decafluoropentane, 1,1,1,2,2,3,4,5,5,6,6,7,7,7-tridecafluoroheptane, iodotrifluoromethane, deuterated hydrocarbons, deuterated hydrofluorocarbons, perfluorocarbons, fluoroethers, brominated compounds, iodated compounds, alcohols, aldehydes, ketones, nitrous oxide (N₂O) and mixtures thereof. In some embodiments, the tracer is a blend containing two or more hydrofluorocarbons, or one hydrofluorocarbon in combination with one or more perfluorocarbons.

The tracer may be added to the compositions of the present invention in predetermined quantities to allow detection of any dilution, contamination or other alteration of the composition.

The additive which may be used with the compositions of the present invention may alternatively be a perfluoropolyether as described in detail in US 2007-0284555, the disclosure of which is incorporated herein by reference in its entirety.

It will be recognized that certain of the additives referenced above as suitable for the non-refrigerant component have been identified as potential refrigerants. However, in accordance with this invention, when these additives are used, they are not present at an amount that would affect the novel and basic characteristics of the refrigerant mixtures of this invention.

In some embodiments, the refrigerant compositions disclosed herein may be prepared by any convenient method to combine the desired amounts of the individual components as is standard in the art. A preferred method is to weigh the desired component amounts and thereafter combine the components in an appropriate vessel. Agitation may be used, if desired.

Compounds and compositions of the present invention have zero ozone depletion potential and low global warming potential (GWP). Additionally, the compounds and compositions of the present invention may have global warming potentials that are less than many hydrofluorocarbon refrigerants currently in use. Therefore, in accordance with the present invention, the compounds and compositions described herein may be useful in methods for producing cooling, producing heating, and transferring heat.

### High Temperature Heat Pumps

The compounds and compositions of the present invention may also be useful in method for producing heating in a high temperature heat pump having a heat exchanger. The method comprises extracting heat from a working fluid, thereby producing a cooled working fluid wherein said working fluid comprises a compound or composition provided herein.

Of note are high temperature heat pumps that may be used to heat air, water, another heat transfer medium or some portion of an industrial chemical process, such as a piece of equipment, storage area or chemical process stream. These high temperature heat pumps can generally operate at condenser temperatures greater than about 55° C. The maximum condenser operating temperature that can be achieved in a high temperature heat pump depends on the working fluid used. This maximum condenser operating temperature is limited by the normal boiling characteristics of the working fluid and, also by the pressure to which the heat pump's compressor can raise the vapor working fluid pressure. This maximum pressure is also related to the working fluid used in the heat pump.

Also of note are heat pumps that are used to produce heating and cooling simultaneously. For instance, a single heat pump unit may produce hot water for domestic use and may also produce cooling for comfort air conditioning in the summer.

Heat pumps, including both flooded evaporator and direct expansion, may be coupled with an air handling and distribution system to provide comfort air conditioning (cooling and dehumidifying the air) and/or heating to residence (single family or attached homes) and large commercial buildings, including hotels, office buildings, hospitals, schools, universities, and the like. In another embodiment, heat pumps may be used to heat water.

It should be noted that for a single component working fluid composition, the composition of the vapor working fluid in the evaporator and condenser is the same as the composition of the liquid working fluid in the evaporator and condenser. In this case, evaporation will occur at a constant temperature. However, if a working fluid blend (or mixture) is used, as in the present invention, the liquid working fluid and the working fluid vapor in the evaporator (or in the condenser) may have different compositions. This may lead to inefficient systems and difficulties in servicing the equipment, thus a single component working fluid is more desirable. An azeotrope or azeotrope-like composition will function essentially as a single component working fluid in a heat pump, such that the liquid composition and the vapor composition are essentially the same reducing any inefficiency that might arise from the use of a non-azeotropic or non-azeotrope-like composition.

Examples of compressors useful in the present invention include dynamic compressors. Of note as examples of dynamic compressors are centrifugal compressors. A centrifugal compressor uses rotating elements to accelerate the working fluid radially, and typically includes an impeller and diffuser housed in a casing. Centrifugal compressors usually take working fluid in at an impeller eye, or central inlet of a circulating impeller, and accelerate it radially outward through passages. Some static pressure rise occurs in the impeller, but most of the pressure rise occurs in the diffuser section of the casing, where velocity is converted to static pressure. Each impeller-diffuser set is a stage of the compressor. Centrifugal compressors are built with from 1 to 12 or more stages, depending on the final pressure desired and the volume of refrigerant to be handled.

The pressure ratio, or compression ratio, of a compressor is the ratio of absolute discharge pressure to the absolute inlet pressure. Pressure delivered by a centrifugal compressor is practically constant over a relatively wide range of capacities. The pressure a centrifugal compressor can develop depends on the tip speed of the impeller. Tip speed is the speed of the impeller measured at its tip and is related to the diameter of the impeller and its revolutions per minute. The tip speed required in a specific application depends on the compressor work that is required to elevate the thermodynamic state of the working fluid from evaporator to condenser conditions. Volumetric flow capacity of a centrifugal compressor is determined by the size of the passages through the impeller. This makes the size of the compressor more dependent on the pressure required than the volumetric flow capacity required.

Also of note as examples of dynamic compressors are axial compressors. A compressor in which the fluid enters and leaves in the axial direction is called an axial flow compressor. Axial compressors are rotating, airfoil- or blade-based compressors in which a working fluid principally flows parallel to the axis of rotation. This is in contrast with other rotating compressors such as centrifugal or mixed-flow compressors in which a working fluid may enter axially but will have a significant radial component on exit. Axial flow compressors produce a continuous flow of compressed gas, and have the benefits of high efficiencies and large mass flow capacity, particularly in relation to their cross-section. They do, however, require several rows of airfoils to achieve large pressure rises making them complex and expensive relative to other designs.

Compressors useful in the present invention also include positive displacement compressors. Positive displacement compressors draw vapor into a chamber, and the chamber decreases in volume to compress the vapor. After being compressed, the vapor is forced from the chamber by further decreasing the volume of the chamber to zero or nearly zero.

An example of positive displacement compressor is a reciprocating compressor. Reciprocating compressors use pistons driven by a crankshaft. They can be either stationary or portable, can be single or multi-staged, and can be driven by electric motors or internal combustion engines. Small reciprocating compressors from 5 to 30 hp are seen in automotive applications and are typically for intermittent duty. Larger reciprocating compressors up to 100 hp are found in large industrial applications. Discharge pressures can range from low pressure to very high pressure (above 5000 psi or 35 MPa).

Also of note as examples of positive displacement compressors are screw compressors. Screw compressors use two meshed rotating positive-displacement helical screws to force the gas into a smaller space. Screw compressors are usually for continuous operation in commercial and industrial application and may be either stationary or portable. Their application can be from 5 hp (3.7 kW) to over 500 hp (375 kW) and from low pressure to very high pressure (above 1200 psi or 8.3 MPa).

Also of note as examples of positive displacement compressors are scroll compressors. Scroll compressors are similar to screw compressors and include two interleaved spiral-shaped scrolls to compress the gas. The output is more pulsed than that of a rotary screw compressor.

The compositions described herein may enable the design and operation of dynamic (e.g. centrifugal) or positive displacement (e.g. screw or scroll) heat pumps for upgrading heat available at low temperatures to meet demands for heating at higher temperatures. The available low temperature heat is supplied to the evaporator and the high temperature heat is extracted at the condenser or working fluid cooler (in a supercritical or transcritical mode). For example, waste heat can be available to be supplied to the evaporator of a heat pump operating at 25° C. at a location (e.g. a hospital) where heat from the condenser, operating at 85° C., can be used to heat water (e.g. for hydronic space heating or other service).

In some cases heat may be available from various other sources (e.g. waste heat from process streams, geothermal heat or solar heat) at temperatures higher than suggested above, while heating at even higher temperatures may be required.

In some embodiments, the heat exchanger is a supercritical working fluid cooler or just working fluid cooler. In some embodiments, the heat exchanger is a condenser.

In some embodiments, provided is a method for producing heating in a high temperature heat pump comprising condensing a vapor working fluid comprising a composition provided herein, in a condenser, thereby producing a liquid working fluid. Of note are methods wherein a vapor working fluid consisting essentially of a composition provided herein is condensed.

The compounds and compositions provided herein may meet the need for a non-flammable high temperature heat pump working fluid with reduced GWP.

Some high temperature heat pumps operated with the compounds and/or compositions provided herein as the working fluid have vapor pressures below the threshold necessitating compliance with provisions of the ASME Boiler and Pressure Vessel Code. Such compounds and/or compositions are desirable for use in high temperature heat pumps. Of note are compositions where the working fluid consists essentially of from about 1 to about 100 weight percent of the fluorinated and perflourinated epoxides provided herein (e.g., compounds of Formula I).

In some embodiments, the method for producing heating in a heat pump having a condenser or working fluid cooler, further comprises passing a heat transfer medium through the condenser or working fluid cooler, whereby cooling (and sometimes condensation) of the working fluid heats the heat transfer medium, and passing the heated heat transfer medium from the condenser or working fluid cooler to a body to be heated.

A heating component for use in a composition for use in heating preferably has a boiling point of −50° C. to 50° C. In some embodiments, the compound of Formula (I) for use as a heating component is selected from (Z)-2,3-difluoro-2-(trifluoromethyl)oxirane, (E)-2,3-difluoro-2-(trifluoromethyl)oxirane, trans-2-fluoro-3-(trifluoromethyl)oxirane, trans-2,3-bis(trifluoromethyl)oxirane, trans-2-(trifluoromethyl)-3-(perfluoroethyl)oxirane, cis-2-fluoro-3-(trifluoromethyl)oxirane, trans-2-fluoro-3-(perfluoropropan-2-yl)oxirane, or a mixture thereof. In some embodiments, the composition for use in heating comprises a compound of Formula (I) selected from (Z)-2,3-difluoro-2-(trifluoromethyl)oxirane, (E)-2,3-difluoro-2-(trifluoromethyl)oxirane, trans-2-fluoro-3-(trifluoromethyl)oxirane, trans-2,3-bis(trifluoromethyl)oxirane, trans-2-(trifluoromethyl)-3-(perfluoroethyl)oxirane, cis-2-fluoro-3-(trifluoromethyl)oxirane, trans-2-fluoro-3-(perfluoropropan-2-yl)oxirane, or a mixture thereof. In some embodiments, the compound of Formula (I) is trans-2,3-bis(trifluoromethyl)oxirane. In some embodiments, the component is one of the preceding compounds of Formula (I), wherein the compound is free of the opposite stereoisomers.

In some embodiments, the composition for use in heating further comprises difluoromethane (HFC-32), 1,1-difluoroethane (HFC-152a), 1,1,1,2-tetrafluoroethane (HFC-134a), 1,1,2,2-tetrafluoroethane (HFC-134), pentafluoroethane (HFC-125), 1,1,1-trifluoroethane (HFC-143a), 1,1,1,2,3,3,3-heptafluoropropane (HFC-227ea), (E)-1,3,3,3-tetrafluoroprop-1-ene (E-HFO-1234ze), 2,3,3,3-tetrafluoroprop-1-ene, 3,3,3-trifluoropropene (HFO-1243zf), (E)-1,1,1,4,4,4-hexafluorobut-2-ene (E-1336mzz), (Z)-1,1,1,4,4,4-hexafluorobut-2-ene (Z-1336mzz), (E)-1-chloro-3,3,3-trifluoropropene (E-1233zd), (Z)-1-chloro-3,3,3-trifluoropropene (Z-1233zd), (Z)-1-chloro-2,3,3,3-tetrafluoropropene (Z-HCFO-1224yd), (E)-1-chloro-2,3,3,3-tetrafluoropropene (E-HCFO-1224yd), (Z)-1,3,3,3-tetrafluoroprop-1-ene (Z-HFO-1234ze), (E)-1,2,3,3-tetrafluoropropene (E-HFO-1234ye), (Z)-1,2,3,3-tetrafluoropropene (Z-HFO-1234ye), (E)-1,1,1,4,4,5,5,5-octafluoro-2-pentene (E-HFO-1438mzz), (Z)-1,1,1,4,4,5,5,5-octafluoro-2-pentene (Z-HFO-1438mzz), (E)-1,3,4,4,4-pentafluoro-3-(trifluoromethyl)but-1-ene (E-HFO-1438ezy), (Z)-1,3,4,4,4-pentafluoro-3-(trifluoromethyl)but-1-ene (Z-HFO-1438ezy), 1,1,1,3,3-pentafluoropropane (HFC-245fa), 1-methoxyheptafluoropropane (HFE-7000), 1,1,1,3,3-pentafluorobutane (HFC-365mfc), 1,1,1,2,2,3,4,5,5,5-decafluoropentane (HFC-4310mee), 1,1,1,2,2,3,3,4,4-nonafluoro-4-methoxybutane (HFE-7100).

A body to be heated may be any space, object or fluid that may be heated. In one embodiment, a body to be heated may be a room, building, or the passenger compartment of an automobile. Alternatively, in others embodiments, a body to be heated may be a secondary loop fluid, heat transfer medium, or heat transfer fluid.

In some embodiments, the heat transfer medium is water and the body to be heated is water. In some embodiments, the heat transfer medium is water and the body to be heated is air for space heating. In some embodiments, the heat transfer medium is an industrial heat transfer liquid and the body to be heated is a chemical process stream.

In some embodiments, the method to produce heating further comprises compressing the working fluid vapor in a dynamic (e.g. axial or centrifugal) compressor or in a positive displacement (e.g. reciprocating, screw or scroll) compressor.

In some embodiments, the method for producing heating in a heat pump having a condenser further comprises passing a fluid to be heated through the condenser, thus heating the fluid. In some embodiments, the fluid is air, and the heated air from the condenser is passed to a space to be heated. In some embodiments, the fluid is a portion of a process stream, and the heated portion is returned to the process.

In some embodiments, the heat transfer medium is selected from water or glycol. The glycol can be, for example, ethylene glycol or propylene glycol. Of particular note are embodiments wherein the heat transfer medium is water and the body to be heated is air for space heating.

In some embodiments, the heat transfer medium is an industrial heat transfer liquid, and the body to be heated is a chemical process stream, which, as used herein, chemical process stream includes process lines and process equipment such as distillation columns. Of note are industrial heat transfer liquids including ionic liquids, various brines such as aqueous calcium chloride or sodium chloride, glycols such as propylene glycol or ethylene glycol, methanol, and other heat transfer media such as those listed in section 4 of the 2006 ASHRAE

Handbook on Refrigeration. In another embodiment, the heat transfer medium is also an epoxide compound or composition as disclosed herein.

In some embodiments, the method for producing heating comprises extracting heat in a flooded evaporator high temperature heat pump. In this method, the liquid working fluid is evaporated to form a working fluid vapor in the vicinity of a first heat transfer medium. The first heat transfer medium is a warm liquid, such as water, which is transported into the evaporator via a pipe from a low temperature heat source. The warm liquid is cooled and is returned to the low temperature heat source or is passed to a body to be cooled, such as a building. The working fluid vapor is then condensed in the vicinity of a second heat transfer medium, which is a chilled liquid which is brought in from the vicinity of a body to be heated (heat sink). The second heat transfer medium cools the working fluid such that it is condensed to form a liquid working fluid. In this method a flooded evaporator heat pump may also be used to heat domestic or service water or a chemical process stream.

In some embodiments, the method for producing heating comprises producing heating in a direct expansion high temperature heat pump. In this method, working fluid liquid is passed through an evaporator and evaporates to produce a working fluid vapor. A first liquid heat transfer medium is cooled by the evaporating working fluid. The first liquid heat transfer medium is passed out of the evaporator to a low temperature heat source or a body to be cooled. The working fluid vapor is then condensed or cooled in the vicinity of a second heat transfer medium, which is a chilled liquid which is brought in from the vicinity of a body to be heated (heat sink). The second heat transfer medium cools the working fluid such that it is condensed to form a liquid working fluid. In this method, a direct expansion heat pump may also be used to heat domestic or service water or a chemical process stream.

In some embodiments of the method for producing heating, the high temperature heat pump includes a compressor which is a centrifugal compressor.

In some embodiments, a method is provided for raising the maximum feasible condenser operating temperature in a high temperature heat pump apparatus comprising charging the high temperature heat pump with a working fluid comprising a composition provided herein.

In some embodiments, the heat pump apparatus comprises an evaporator, a compressor, a condenser (or working fluid cooler) and a pressure reduction device, all of which are in fluid communication in the order listed and through which a working fluid flows from one component to the next in a repeating cycle.

In some embodiments, the heat pump apparatus comprises (a) an evaporator through which a working fluid flows and is evaporated; (b) a compressor in fluid communication with the evaporator that compresses the evaporated working fluid to a higher pressure; (c) a condenser in fluid communication with the compressor through which the high pressure working fluid vapor flows and is condensed; and (d) a pressure reduction device in fluid communication with the condenser wherein the pressure of the condensed working fluid is reduced and said pressure reduction device further being in fluid communication with the evaporator such that the working fluid may repeat flow through components (a), (b), (c) and (d) in a repeating cycle; wherein the working fluid comprises a composition provided herein.

For high temperature condenser operation (e.g., associated with high temperature lifts and high compressor discharge temperatures) formulations of working fluid (e.g. a composition provided herein) and lubricants with high thermal stability (e.g., possibly in combination with oil cooling or other mitigation approaches) will be advantageous.

For high temperature condenser operation (e.g., associated with high temperature lifts and high compressor discharge temperatures) use of magnetic centrifugal compressors (e.g., Danfoss-Turbocor type) that do not require the use of lubricants will be advantageous.

For high temperature condenser operation (e.g., associated with high temperature lifts and high compressor discharge temperatures) use of compressor materials (e.g., shaft seals and the like) with high thermal stability may also be required.

In some embodiments, certain refrigeration, air-conditioning, or heat pump system additives may optionally be added, as desired, to the working fluids as disclosed herein (i.e., a composition provided herein) in order to enhance performance and system stability. These additives are known in the field of refrigeration and air-conditioning, and include, but are not limited to, anti-wear agents, extreme pressure lubricants, corrosion and oxidation inhibitors, metal surface deactivators, free radical scavengers, and foam control agents. In general, these additives may be present in the working fluids in small amounts relative to the overall composition. Typically concentrations of from less than about 0.1 weight percent to as much as about 3 weight percent of each additive are used. These additives are selected on the basis of the individual system requirements. These additives include members of the triaryl phosphate family of EP (extreme pressure) lubricity additives, such as butylated triphenyl phosphates (BTPP), or other alkylated triaryl phosphate esters, e.g. Syn-O-Ad 8478 from Akzo Chemicals, tricresyl phosphates and related compounds. Additionally, the metal dialkyl dithiophosphates (e.g., zinc dialkyl dithiophosphate (or ZDDP); Lubrizol 1375 and other members of this family of chemicals may be used in compositions of the present invention. Other antiwear additives include natural product oils and asymmetrical polyhydroxyl lubrication additives, such as Synergol TMS (International Lubricants). Similarly, stabilizers such as antioxidants, free radical scavengers, and water scavengers may be employed. Compounds in this category can include, but are not limited to, butylated hydroxy toluene (BHT), epoxides, and mixtures thereof. Corrosion inhibitors include dodecyl succinic acid (DDSA), amine phosphate (AP), oleoyl sarcosine, imidazone derivatives and substituted sulfphonates. Metal surface deactivators include areoxalyl bis(benzylidene) hydrazide (CAS reg no. 6629-10-3), N,N′-bis(3,5-di-tert-butyl-4-hydroxyhydrocinnamoylhydrazine (CAS reg no. 32687-78-8), 2,2,′-oxamidobis-ethyl-(3,5-di-tert-butyl-4-hydroxyhydrocinnamate (CAS reg no. 70331-94-1), N,N′-(disalicyclidene)-1,2-diaminopropane (CAS reg no. 94-91-7) and ethylenediaminetetra-acetic acid (CAS reg no. 60-00-4) and its salts, and mixtures thereof.

Of note are stabilizers and ionic liquid stabilizers comprising at least one compound selected from the group of stabilizers and ionic liquid stabilizers described above under “Refrigerants”.

### Organic Rankine Cycles

The compounds and compositions of the invention may also be useful in processes for converting heat to mechanical work in a power cycle (e.g., an organic Rankine cycle). The power cycle includes the steps of heating a working fluid with a heat source to a temperature sufficient to pressurize the working fluid and causing the pressurized working fluid to perform mechanical work. In some embodiments, the process may utilize a sub-critical power cycle, trans-critical power cycle, or a super-critical power cycle.

An Organic Rankine Cycle (ORC) system is named for its use of organic working fluids that enable such a system to capture heat from low temperature heat sources such as geothermal heat, biomass combustors, industrial waste heat, and the like. The captured heat maybe converted by the ORC system into mechanical work and/or electricity. Organic working fluids are selected for their liquid-vapor phase change characteristics, such as having a lower boiling temperature than water.

A typical ORC system includes an evaporator for absorbing heat to evaporate a liquid organic working fluid into a vapor, an expansion device, such as a turbine, through which the vapor expands, a condenser to condense the expanded vapor back into a liquid, and a compressor or liquid pump to cycle the liquid working fluid back through the evaporator to repeat the cycle. As the organic fluid vapor expands through the turbine, it turns the turbine which in turn rotates an output shaft. The rotating output shaft may be further connected through mechanical linkage to produce mechanical energy or turn a generator to produce electricity.

The organic working fluid undergoes the following cycle in an ORC system: near adiabatic pressure rise through the compressor, near isobaric heating through the evaporator, near adiabatic expansion in the expander, and near isobaric heat rejection in the condenser.

Working fluids for use in ORC systems exhibit thermodynamic properties that are suitable for use with low temperature heat sources, have non-flammable characteristics, and no Ozone Depletion Potential (ODP).

In some embodiments, the present invention relates to a process of using a working fluid (e.g., a compound or composition provided herein) comprising a fluorinated or perfluorinated compound provided herein to convert heat to mechanical work by using a sub-critical power cycle. The ORC system is operating in a sub-critical cycle when the working fluid receives heat at a pressure lower than the critical pressure of the working fluid and the working fluid remains below its critical pressure throughout the entire cycle. This process comprises the following steps: (a) compressing a liquid working fluid to a pressure below its critical pressure; (b) heating the compressed liquid working fluid from step (a) using heat supplied by the heat source to form a vapor working fluid; (c) expanding the vapor working fluid from step (b) in an expansion device to generate mechanical work; (d) cooling the expanded working fluid from step (c) to form a cooled liquid working fluid; and (e) cycling the cooled liquid working fluid from step (d) to step (a) to repeat the cycle.

In the case of sub-critical cycle operations, most heat supplied to the working fluid is supplied during evaporation of the working fluid. As a result, when the working fluid consists of a single fluid component or when the working fluid is a near-azeotropic multicomponent fluid blend, the working fluid temperature is essentially constant during transfer of heat from the heat source to the working fluid.

In contrast with the subcritical cycle, the working fluid temperature can vary when the fluid is heated isobarically without phase change at a pressure above its critical pressure. Accordingly, when the heat source temperature varies, use of a fluid above its critical pressure to extract heat from a heat source allows better matching between the heat source temperature and the working fluid temperature compared to the case of sub-critical heat extraction. As a result, efficiency of the heat exchange system between a temperature-varying heat source and a single component or near-azeotropic working fluid in a super-critical cycle or a trans-critical cycle is often higher than that of a sub-critical cycle (see Chen, et al., Energy, 36, (2011) 549-555 and references therein).

In another embodiment, the present invention relates to a process of using a working fluid provided herein to convert heat energy to mechanical work by using a trans-critical power cycle. The ORC system is operating as a trans-critical cycle when the working fluid receives heat at a pressure higher than the critical pressure of the working fluid. In a trans-critical cycle, the working fluid does not remain at a pressure higher than its critical pressure throughout the entire cycle. This process comprises the following steps: (a) compressing a liquid working fluid to a pressure above the working fluid's critical pressure; (b) heating the compressed working fluid from step (a) using heat supplied by the heat source; (c) expanding the heated working fluid from step (b) to lower the pressure of the working fluid below its critical pressure to generate mechanical work; (d) cooling the expanded working fluid from step (c) to form a cooled liquid working fluid; and (e) cycling the cooled liquid working fluid from step (d) to step (a) to repeat the cycle.

In the first step of the trans-critical power cycle system, described above, the working fluid in liquid phase is compressed to above its critical pressure. In a second step, said working fluid is passed through a heat exchanger to be heated to a higher temperature before the fluid enters the expander wherein the heat exchanger is in thermal communication with said heat source. The heat exchanger receives heat energy from the heat source by any known means of thermal transfer. The ORC system working fluid circulates through the heat supply heat exchanger where the fluid gains heat.

In the next step, at least a portion of the heated working fluid is removed from the heat exchanger and is routed to the expander where fluid expansion results in conversion of at least portion of the heat energy content of the working fluid into mechanical energy, such as shaft energy. The pressure of the working fluid is reduced to below the critical pressure of the working fluid, thereby producing vapor phase working fluid.

In the next step, the working fluid is passed from the expander to a condenser, wherein the vapor phase working fluid is condensed to produce liquid phase working fluid. The above steps form a loop system and can be repeated many times.

Additionally, for a trans-critical power cycle, there are several different modes of operation. In one mode of operation, in the first step of a trans-critical power cycle, the working fluid is compressed above the critical pressure of the working fluid substantially isentropically. In the next step, the working fluid is heated under a substantially constant pressure (isobaric) condition to above its critical temperature. In the next step, the working fluid is expanded substantially isentropically at a temperature that maintains the working fluid in the vapor phase. At the end of the expansion the working fluid is a superheated vapor at a temperature below its critical temperature. In the last step of this cycle, the working fluid is cooled and condensed while heat is rejected to a cooling medium. During this step the working fluid is condensed to a liquid. The working fluid could be subcooled at the end of this cooling step.

In another mode of operation of a trans-critical ORC power cycle, in the first step, the working fluid is compressed above the critical pressure of the working fluid, substantially isentropically. In the next step the working fluid is then heated under a substantially constant pressure condition to above its critical temperature, but only to such an extent that in the next step, when the working fluid is expanded substantially isentropically, and its temperature is reduced, the working fluid is sufficiently close to being a saturated vapor that partial condensation or misting of the working fluid may occur. At the end of this step, however, the working fluid is still a slightly superheated vapor. In the last step, the working fluid is cooled and condensed while heat is rejected to a cooling medium. During this step the working fluid is condensed to a liquid. The working fluid could be subcooled at the end of this cooling/condensing step.

In another mode of operation of a trans-critical ORC power cycle, in the first step, the working fluid is compressed above the critical pressure of the working fluid, substantially isentropically. In the next step, the working fluid is heated under a substantially constant pressure condition to a temperature either below or only slightly above its critical temperature. At this stage, the working fluid temperature is such that when the working fluid is expanded substantially isentropically in the next step, the working fluid is partially condensed. In the last step, the working fluid is cooled and fully condensed and heat is rejected to a cooling medium. The working fluid may be subcooled at the end of this step.

While the above embodiments for a trans-critical ORC cycle show substantially isentropic expansions and compressions, and substantially isobaric heating or cooling, other cycles wherein such isentropic or isobaric conditions are not maintained but the cycle is nevertheless accomplished, is within the scope of the present invention.

Another embodiment of the present invention relates to a process of using a working fluid comprising a composition provided herein to convert heat energy to mechanical work by using a super-critical power cycle. An ORC system is operating as a super-critical cycle when the working fluid used in the cycle is at pressures higher than its critical pressure throughout the cycle. The working fluid of a super-critical ORC does not pass through a distinct vapor-liquid two-phase transition as in a sub-critical or trans-critical ORC. This method comprises the following steps: (a) compressing a working fluid from a pressure above its critical pressure to a higher pressure; (b) heating the compressed working fluid from step (a) using heat supplied by the heat source; (c) expanding the heated working fluid from step (b) to lower the pressure of the working fluid to a pressure above its critical pressure and generate mechanical work; (d) cooling the expanded working fluid from step (c) to form a cooled working fluid above its critical pressure; and (e) cycling the cooled working fluid from step (d) to step (a) for compression.

Typically for super-critical cycles, the temperature to which the working fluid is heated using heat from the heat source is in the range of from about 190° C. to about 300° C., preferably from about 200° C. to about 250° C., more preferably from about 200° C. to 225° C. The pressure of the working fluid in the expander is reduced from the expander inlet pressure to the expander outlet pressure. Typical expander inlet pressures for super-critical cycles are within the range of from about 2 MPa to about 7 MPa, preferably from about 2 MPa to about 5 MPa, and more preferably from about 3 MPa to about 4 MPa. Typical expander outlet pressures for super-critical cycles are within about 0.01 MPa above the critical pressure.

The working fluids of the present invention (i.e., compositions provided herein) may be used in ORC systems to generate mechanical work from heat extracted or received from relatively low temperature heat sources such as low pressure steam, industrial waste heat, solar energy, geothermal hot water, low-pressure geothermal steam (primary or secondary arrangements), or distributed power generation equipment utilizing fuel cells or prime movers such as turbines, micro-turbines, or internal combustion engines. One source of low-pressure steam could be the system known as a binary geothermal Rankine cycle. Large quantities of low-pressure steam can be found in numerous locations, such as in fossil fuel powered electrical generating power plants.

A working fluid component for use in converting heat into mechanical work preferably has a boiling point of 0° C. to 150° C., more preferably, 10° C. to 75° C. In some embodiments, the compound for use in an ORC system (e.g., as a working fluid component to convert heat into mechanical work) is selected from (Z)-2,3-difluoro-2-(trifluoromethyl)oxirane, (E)-2,3-difluoro-2-(trifluoromethyl)oxirane, cis-2-fluoro-3-(trifluoromethyl)oxirane, trans-2-fluoro-3-(trifluoromethyl)oxirane, trans-2,3-bis(trifluoromethyl)oxirane, cis-2,3-bis(trifluoromethyl)oxirane, trans-2-(trifluoromethyl)-3-(perfluoroethyl)oxirane, trans-2-(trifluoromethyl)-3-(perfluoropropan-2-yl)oxirane, trans-2,3-bis(perfluoropropyl)oxirane, trans-2-(perfluorobutyl)-3-(perfluoroethyl)oxirane, trans-2,3-bis(perfluorobutyl)oxirane, (Z)-2-(2,2,2-Trifluoroethoxy)-3-fluoro-2-(trifluoromethyl)-3-(perfluoroethyl)oxirane, (E)-2-(2,2,2-Trifluoroethoxy)-3-fluoro-2-(trifluoromethyl)-3-(perfluoroethyl)oxirane, cis-2,3-dichloro-2,3-bis(trifluoromethyl)oxirane, trans-2,3-dichloro-2,3-bis(trifluoromethyl)oxirane, trans-2-fluoro-3-(perfluoropropan-2-yl)oxirane, cis-2-fluoro-3-(perfluoropropan-2-yl)oxirane, cis-2,2,3,3,4,4-hexafluoro-6-oxa-bicyclo[3.1.0]hexane, cis-2,2,3,3-tetrafluoro-5-oxabicyclo[2.1.0]pentane, cis-2,3-difluoro-2-(perfluoroethyl)-3-(perfluoropropyl)oxirane, trans-2,3-difluoro-2-(perfluoroethyl)-3-(perfluoropropyl)oxirane, cis-2,3-difluoro-2-(trifluoromethyl)-3-(perfluoropentyl)oxirane, and trans-2,3-difluoro-2-(trifluoromethyl)-3-(perfluoropentyl)oxirane, or a mixture thereof. In some embodiments, the component is one of the preceding compounds of Formula (I), wherein the compound is free of the opposite stereoisomers.

In some embodiments, the composition further comprises difluoromethane, 1,1-difluoroethane, 1,1,1,2-tetrafluoroethane, pentafluoroethane, 1,1,1,2,3,3,3-heptafluoropropane, (E)-1,3,3,3-tetrafluoroprop-1-ene (E-HFO-1234ze), 2,3,3,3-tetrafluoroprop-1-ene (HFO-1234yf), (E)-1,1,1,4,4,4-hexafluorobut-2-ene (E-HFO-1336mzz), (Z)-1,1,1,4,4,4-hexafluorobut-2-ene (Z-1336mzz), (E)-1-chloro-3,3,3-trifluoropropene (E-1233zd), (Z)-1-chloro-3,3,3-trifluoropropene (Z-1233zd), (Z)-1-chloro-2,3,3,3-tetrafluoropropene (Z-HCFO-1224yd), (E)-1-chloro-2,3,3,3-tetrafluoropropene (E-HCFO-1224yd), (Z)-1,3,3,3-tetrafluoroprop-1-ene (Z-HFO-1234ze), (E)-1,2,3,3-tetrafluoropropene (E-HFO-1234ye), (Z)-1,2,3,3-tetrafluoropropene (Z-HFO-1234ye), (E)-1,1,1,4,4,5,5,5-octafluoro-2-pentene (E-HFO-1438mzz), (Z)-1,1,1,4,4,5,5,5-octafluoro-2-pentene (Z-HFO-1438mzz), (E)-1,3,4,4,4-pentafluoro-3-(trifluoromethyl)but-1-ene (E-HFO-1438ezy), (Z)-1,3,4,4,4-pentafluoro-3-(trifluoromethyl)but-1-ene (Z-HFO-1438ezy), 1,1,1,3,3-pentafluoropropane (HFC-245fa), 1-methoxyheptafluoropropane (HFE-7000), 1,1,1,3,3-pentafluorobutane (HFC-365mfc), 1,1,1,2,2,3,4,5,5,5-decafluoropentane (HFC-4310mee), 1,1,1,2,2,3,3,4,4-nonafluoro-4-methoxybutane (HFE-7100).

In some embodiments, the composition for use in an ORC system comprises about 0.1% to 100%, about 0.1% to about 99%, about 1% to about 99%, about 10% to 99%, about 10% to about 99%, about 20% to about 99%, about 30% to about 90%, about 40% to about 99%, about 50% to about 99%, about 60% to about 99%, about 70% to about 99%, about 80% to about 99%, about 90% to about 99%, about 40% to about 90%, about 50% to about 90%, about 60% to about 90%, about 70% to about 90%, about 60% to about 80%, or about 50% to about 70% w/w of the compound of Formula (I) or a mixture of compounds of Formula (I).

Other sources of heat include waste heat recovered from gases exhausted from mobile internal combustion engines (e.g. truck or rail or marine diesel engines), waste heat from exhaust gases from stationary internal combustion engines (e.g. stationary diesel engine power generators), waste heat from fuel cells, heat available at combined heating, cooling and power or district heating and cooling plants, waste heat from biomass fueled engines, heat from natural gas or methane gas burners or methane-fired boilers or methane fuel cells (e.g. at distributed power generation facilities) operated with methane from various sources including biogas, landfill gas and coal-bed methane, heat from combustion of bark and lignin at paper/pulp mills, heat from incinerators, heat from low pressure steam at conventional steam power plants (to drive “bottoming” Rankine cycles), and geothermal heat.

In one embodiment of the Rankine cycles of this invention, geothermal heat is supplied to the working fluid circulating above ground (e.g. binary cycle geothermal power plants). In another embodiment of the Rankine cycles of this invention, a novel working fluid composition of this invention is used both as the Rankine cycle working fluid and as a geothermal heat carrier circulating underground in deep wells with the flow largely or exclusively driven by temperature-induced fluid density variations, known as “the thermosyphon effect” (e.g. see Davis, A. P. and E. E. Michaelides: “Geothermal power production from abandoned oil wells”, Energy, 34 (2009) 866-872; Matthews, H. B. U.S. Pat. No. 4,142,108-Feb. 27, 1979)

Other sources of heat include solar heat from solar panel arrays including parabolic solar panel arrays, solar heat from concentrated solar power plants, heat removed from photovoltaic (PV) solar system to cool the PV system to maintain a high PV system efficiency.

In other embodiments, the present invention also uses other types of ORC system, for example, small scale (e.g. 1-500 kW, preferably 5-250 kW) Rankine cycle system using micro-turbines or small size positive displacement expanders (e.g. Tahir, Yamada and Hoshino: “Efficiency of compact organic Rankine cycle system with rotary-vane-type expander for low-temperature waste heat recovery”, Intl J. of Civil and Environ. Eng 2:1 2010), combined, multistage, and cascade Rankine Cycles, and Rankine Cycle system with recuperators to recover heat from the vapor exiting the expander.

Other sources of heat include at least one operation associated with at least one industry selected from the group consisting of: marine shipping, oil refineries, petrochemical plants, oil and gas pipelines, chemical industry, commercial buildings, hotels, shopping malls, supermarkets, bakeries, food industries, restaurants, paint curing ovens, furniture making, plastics molders, cement kilns, lumber kilns, calcining operations, steel industry, glass industry, foundries, smelting, air-conditioning, refrigeration, and central heating.

### Fire Extinguishing and Fire Suppression

The compounds and compositions provided herein may be used alone or in admixture with each other or in blends with other fire extinguishing agents for use in methods of fire extinguishing or fire suppression. Among the other agents with which the fluorinated and perfluorinated epoxides of this invention may be blended are chlorine and/or bromine containing compounds such as Halon 1301 (CF₃Br), Halon 1211 (CF₂BrCl), Halon 2402 (CF₂BrCF₂Br), Halon 251 (CF₃CF₂Cl) and CF₃CHFBr.

In some embodiments, the present application provides a process for extinguishing or suppressing a flame comprising dispensing a compound or composition provided herein at said flame.

Preferably, the fire suppression or fire extinguishing agent has a boiling point of −100° C. to about 45-50° C. Preferably, in a streaming application, the compound has a boiling point of −20° C. to 50° C.

In some embodiments, the compound for use in fire extinguishing or fire suppression is (E)-2,3-difluoro-2-(trifluoromethyl)oxirane, (Z)-2,3-difluoro-2-(trifluoromethyl)oxirane, trans-2-fluoro-3-(trifluoromethyl)oxirane, trans-2,3-bis(trifluoromethyl)oxirane, trans-2-(trifluoromethyl)-3-(perfluoroethyl)oxirane, cis-2-fluoro-3-(trifluoromethyl)oxirane, trans-2-fluoro-3-(perfluoropropan-2-yl)oxirane, or a mixture thereof. In some embodiments, the component is one of the preceding compounds of Formula (I), wherein the compound is free of the opposite stereoisomers.

In some embodiments, the composition provided herein for use in fire extinguishing or fire suppression comprises (a) a fluoroepoxide of Formula (I) selected from (E)-2,3-difluoro-2-(trifluoromethyl)oxirane, (Z)-2,3-difluoro-2-(trifluoromethyl)oxirane, trans-2-fluoro-3-(trifluoromethyl)oxirane, trans-2,3-bis(trifluoromethyl)oxirane, trans-2-(trifluoromethyl)-3-(perfluoroethyl)oxirane, cis-2-fluoro-3-(trifluoromethyl)oxirane, trans-2-fluoro-3-(perfluoropropan-2-yl)oxirane, or a mixture thereof; and (b) one or more of 2-bromo-1,1,1-trifluoro-2-propene, E-1,2-dichloro-1,2-difluoroethylene, Z-1,2-dichloro-1,2-difluoroethylene, E-1-chloro-3,3,3-trifluoropropene, Z-1-chloro-3,3,3-trifluoropropene, E-1,1,1,4,4,4-hexafluoro-2-butene, Z-1,1,1,4,4,4-hexafluoro-2-butene, perfluoroethyl perfluoroisopropyl ketone (F-ethyl isopropyl ketone), E-1,3,4,4,4-pentafluoro-3-(trifluoromethyl)-1-butene), E-1,2,3,3,3-pentafluoropropene, Z-1,2,3,3,3-pentafluoropropene, E-1-chloro-2,3,3,3-tetrafluoropropene, Z-1-chloro-2,3,3,3-tetrafluoropropene, CF₃I, carbon dioxide, nitrogen, and argon.

In some embodiments, the composition provided herein for use in fire extinguishing or fire suppression comprises a fluoroepoxide of Formula (I) which is trans-2,3-bis(trifluoromethyl)oxirane.

In some embodiments, the composition provided herein for use in fire extinguishing or fire suppression comprises (a) a fluoroepoxide of Formula (I) which is trans-2,3-bis(trifluoromethyl)oxirane and (b) one or more of 2-bromo-1,1,1-trifluoro-2-propene, E-1,2-dichloro-1,2-difluoroethylene, Z-1,2-dichloro-1,2-difluoroethylene, E-1-chloro-3,3,3-trifluoropropene, Z-1-chloro-3,3,3-trifluoropropene, E-1,1,1,4,4,4-hexafluoro-2-butene, Z-1,1,1,4,4,4-hexafluoro-2-butene, perfluoroethyl perfluoroisopropyl ketone (F-ethyl isopropyl ketone), E-1,3,4,4,4-pentafluoro-3-(trifluoromethyl)-1-butene), E-1,2,3,3,3-pentafluoropropene, Z-1,2,3,3,3-pentafluoropropene, E-1-chloro-2,3,3,3-tetrafluoropropene, Z-1-chloro-2,3,3,3-tetrafluoropropene, CF₃I, carbon dioxide, nitrogen, and argon.

In some embodiments, the fire extinguishing or fire suppression composition provided herein comprises about 5% to 100%, about 0.1% to 100%, about 0.1% to about 99%, about 1% to about 99%, about 10% to 99%, about 10% to about 99%, about 20% to about 99%, about 30% to about 90%, about 40% to about 99%, about 50% to about 99%, about 60% to about 99%, about 70% to about 99%, about 80% to about 99%, about 90% to about 99%, about 40% to about 90%, about 50% to about 90%, about 60% to about 90%, about 70% to about 90%, about 60% to about 80%, or about 50% to about 70% w/w of the compound of Formula (I) or a mixture of compounds of Formula (I).

The present application further provides a method of reducing the flammability of a fluid, comprising adding a compound or composition provided herein to the fluid

The maximum amount of the fluorinated and perfluorinated epoxides to be employed will be governed by matters of economics and potential toxicity to living things. About 15% (v/v) of the fluorinated or perfluorinated epoxides in air provides a convenient maximum concentration for use of fluorinated or perfluorinated epoxide and mixtures and blends thereof in occupied areas. Concentrations above 15% (v/v) in air may be employed in unoccupied areas, with the exact level being determined by the particular combustible material, the fluorinated or perfluorinated epoxide (or mixture or blend thereof) chosen and the conditions of combustion. The preferred concentration of the fluorinated or perfluorinated epoxides, mixtures and blends in accordance with this invention lies in the range of about 5 to 10% (v/v) in air.

The present application further provides a system for preventing or suppressing a flame comprising a vessel containing a fire extinguishing or fire suppression compound or composition provided herein and a nozzle to dispense said compound or composition toward an anticipated or actual location of said flame.

Thus, these agents may be used in a total flooding fire extinguishing system in which the agent is introduced to an enclosed region (e.g., a room or other enclosure) surrounding a fire at a concentration sufficient to extinguish the fire. In accordance with a total flooding system apparatus, equipment or even rooms or enclosures may be provided with a source of agent and appropriate piping, valves, and controls so as automatically and/or manually to be introduced at appropriate concentrations in the event that fire should break out. Thus, as is known to those skilled in the art, the fire extinguishant may be pressurized with nitrogen or other inert gas at up to about 1200 psig at ambient conditions.

Alternatively, the fluorinated and perfluorinated epoxides of the present invention may be applied to a fire through the use of conventional portable fire extinguishing equipment. It is usual to increase the pressure in portable fire extinguishers with nitrogen or other inert gasses in order to ensure that the agent is completely expelled from the extinguisher. Fire extinguishing systems in accordance with this invention may be conveniently pressurized at any desirable pressure up to about 600 psig at ambient conditions.

A further aspect provides methods of suppressing a flame, said methods comprising contacting a flame with a fluid comprising a compound or composition of the present disclosure. Any suitable methods for contacting the flame with the present composition may be used. For example, an inventive composition of the present disclosure may be sprayed, poured, and the like onto the flame, or at least a portion of the flame may be immersed in the flame suppression composition. In light of the teachings herein, those of skill in the art will be readily able to adapt a variety of conventional apparatus and methods of flame suppression for use in the present disclosure.

In some embodiments, the present application provides methods of extinguishing or suppressing a fire in a total-flood application, comprising providing a compound provided herein or a composition provided herein comprising a fluorinated or perfluorinated epoxide of the invention; disposing the composition in a pressurized discharge system; and discharging the composition into an area to extinguish or suppress fires in that area.

Another embodiment provides methods of inerting a space to prevent a fire or explosion comprising providing an agent comprising an inventive composition of the present disclosure;

disposing the agent in a pressurized discharge system; and discharging the agent into the space to prevent a fire or explosion from occurring.

The term “extinguishment” is usually used to denote complete elimination of a fire; whereas, “suppression” is often used to denote reduction, but not necessarily total elimination, of a fire or explosion. As used herein, terms “extinguishment” and “suppression” will be used interchangeably. There are four general types of halocarbon fire and explosion protection applications:
1) In total-flood fire extinguishment and/or suppression applications, the agent is discharged into a space to achieve a concentration sufficient to extinguish or suppress an existing fire. Total flooding use includes protection of enclosed, potentially occupied spaces such, as computer rooms as well as specialized, often unoccupied spaces such as aircraft engine nacelles and engine compartments in vehicles.
2) In streaming applications, the agent is applied directly onto a fire or into the region of a fire. This is usually accomplished using manually operated wheeled or portable units. A second method, included as a streaming application, uses a “localized” system, which discharges the agent toward a fire from one or more fixed nozzles. Localized systems may be activated either manually or automatically.
3) In explosion suppression, an inventive composition of the present disclosure is discharged to suppress an explosion that has already been initiated. The term “suppression” is normally used in this application because the explosion is usually self-limiting. However, the use of this term does not necessarily imply that the explosion is not extinguished by the agent. In this application, a detector is usually used to detect an expanding fireball from an explosion, and the agent is discharged rapidly to suppress the explosion. Explosion suppression is used primarily, but not solely, in defense applications.
4) In inertion, an inventive composition of the present disclosure is discharged into a space to prevent an explosion or a fire from being initiated. Often, a system similar or identical to that used for total-flood fire extinguishment or suppression is used. Usually, the presence of a dangerous condition (for example, dangerous concentrations of flammable or explosive gases) is detected, and the inventive composition of the present disclosure is then discharged to prevent the explosion or fire from occurring until the condition can be remedied.

The extinguishing method can be carried out by introducing the composition into an enclosed area surrounding a fire. Any of the known methods of introduction can be utilized provided that appropriate quantities of the composition are metered into the enclosed area at appropriate intervals. For example, a composition can be introduced by streaming, e.g., using conventional portable (or fixed) fire extinguishing equipment; by misting; or by flooding, e.g., by releasing (using appropriate piping, valves, and controls) the composition into an enclosed area surrounding a fire. The composition can optionally be combined with an inert propellant provided herein including but not limited to, nitrogen, argon, decomposition products of glycidyl azide polymers or carbon dioxide, to increase the rate of discharge of the composition from the streaming or flooding equipment utilized.

Preferably, the extinguishing process involves introducing an inventive composition of the present disclosure to a fire or flame in an amount sufficient to extinguish the fire or flame. One skilled in this field will recognize that the amount of flame suppressant needed to extinguish a particular fire will depend upon the nature and extent of the hazard. When the flame suppressant is to be introduced by flooding, cup burner test data are useful in determining the amount or concentration of flame suppressant required to extinguish a particular type and size of fire.

Laboratory tests useful for determining effective concentration ranges of an inventive composition when used in conjunction with extinguishing or suppressing a fire in a total-flood application or fire inertion are described, for example, in U.S. Pat. No. 5,759,430.

The present application further provides sprayable compositions. In some embodiments, the sprayable composition comprises one or more of the fluorinated or perfluorinated epoxides provided herein and one or more additional agents. In some embodiments, the sprayable composition contains a bitterant to discourage inhalation abuse of the aerosol composition. Suitable bitterants include but are not limited to denatonium benzoate. In an additional embodiment, the composition contains a solvent to aid in dissolution of aerosol ingredients. Suitable solvents include but are not limited to acetone, water, and alcohols (including but not limited to methanol, ethanol, n-propanol and isopropanol).

### Propellants

The present invention further provides the fluorinated and perfluorinated compounds and compositions provided herein for use as a propellant in sprayable composition. Additionally, the present invention relates to a sprayable composition comprising an inventive composition as described herein. The active ingredient to be sprayed together with inert ingredients, solvents, and other materials may also be present in a sprayable composition. Preferably, the sprayable composition is an aerosol. Suitable active materials to be sprayed include, without limitations, cosmetic materials, such as deodorants, perfumes, hair sprays, cleaners, and polishing agents as well as medicinal materials such as anti-asthma and anti-halitosis medications.

The present invention further relates to a process for producing aerosol products comprising the step of adding a compound or composition as described herein to active ingredients in an aerosol container, wherein said compound or composition functions as a propellant.

Suitability of a compound for use as an aerosol propellant component involves consideration of its boiling point, solvency properties, toxicity, and stability. For example, fluorocarbon (FC) propellants include CFC-12, and CFC-11 (typically in combination with CFC-12), PFC-C-318, CFC-114, CFC-113, HFC-134a, HFC-134, HFC-152a, HFO-1234ze, and HFC-1234yf. These FC propellants are discussed, for example, in Chapter 3 “Fluorocarbon Propellants—Current and Alternative,” of Handbook of Aerosol Technology, PA Sanders, Kreiger Publ Co., FL, 2nd ed. 1987, the disclosure of which is incorporated herein by reference in its entirety.

In some embodiments, the compounds and compositions provided herein may be useful as a co-propellant in a sprayable composition.

In some embodiments, the present application provides a sprayable composition comprising a propellant component and a co-propellant component comprising a compound of Formula (I) as described herein.

In some embodiments, the compound provided herein (e.g., the compound of Formula (I)) useful as a co-propellant in a sprayable composition) is (Z)-2,3-difluoro-2-(trifluoromethyl)oxirane, (E)-2,3-difluoro-2-(trifluoromethyl)oxirane, trans-2-fluoro-3-(trifluoromethyl)oxirane, trans-2,3-bis(trifluoromethyl)oxirane, cis-2,3-bis(trifluoromethyl)oxirane, trans-2-(trifluoromethyl)-3-(perfluoroethyl)oxirane, trans-2-(trifluoromethyl)-3-(perfluoropropan-2-yl)oxirane, trans-2,3-bis(perfluoropropyl)oxirane, trans-2-(perfluorobutyl)-3-(perfluoroethyl)oxirane, trans-2,3-bis(perfluorobutyl)oxirane, 2-(2,2,2-Trifluoroethoxy)-3-fluoro-2-(trifluoromethyl)-3-(perfluoroethyl)oxirane, 2,3-dichloro-2,3-bis(trifluoromethyl)oxirane, trans-2-fluoro-3-(perfluoropropan-2-yl)oxirane, cis-2-fluoro-3-(perfluoropropan-2-yl)oxirane, 2,2,3,3,4,4-hexafluoro-6-oxa-bicyclo[3.1.0]hexane, 2,3,3-tetrafluoro-5-oxabicyclo[2.1.0]pentane, 2,3-difluoro-2-(perfluoroethyl)-3-(perfluoropropyl)oxirane, 2,3-difluoro-2-(trifluoromethyl)-3-(perfluoropentyl)oxirane, and trans-2,3-bis(perfluorobutyl)oxirane. In some embodiments, the component is one of the preceding compounds of Formula (I), wherein the compound is free of the opposite stereoisomers.

In some embodiments, the sprayable composition further comprises a propellant component selected from carbon dioxide, difluoromethane (CF₂H₂, HFC-32), trifluoromethane (CF₃H, HFC-23), difluoroethane (CHF₂CH₃, HFC-152a), trifluoroethane (CH₃CF₃, HFC-143a; or CHF₂CH₂F, HFC-143), tetrafluoroethane (CF₃CH₂F, HFC-134a; or CF₂HCF₂H, HFC-134), pentafluoroethane (CF₃CF₂H, HFC-125), 1,3,3,3-tetrafluoro-1-propene (HFO-1234ze), 2,3,3,3-tetrafluoro-1-propene (HFO-1234yf), 1,2,3,3,3-pentafluoropropene (HFO-1225ye), 1,1,3,3,3-pentafluoropropene (HFO-1225ze) and hydrocarbons (e.g., propane, butanes, or pentanes) or dimethyl ether, or mixtures thereof.

In some embodiments, the sprayable composition further comprises one or more additional co-propellant components selected from dichlorodifluoromethane (CFC-12), trichlorofluoromethane (CFC-11), octafluorocyclobutane (C-318), 1,2-dichloro-1,1,2,2-tetrafluoroethane (CFC-114), 1,1,2-trichloro-1,2,2-trifluoroethane (CFC-113), dimethoxyethane (DME), ethers (e.g., dimethyl ether or diethyl ether), hydrofluorocarbon (e.g., difluoromethane (HFC-32), 1,1,1,2-tetrafluoroethane (HFC-134a), HFC-134a, 1,1,2,2-tetrafluoroethane (HFC-134), 1,1-difluoroethan (HFC-152a), 1,1,1,3,3,3-hexafluoropropane (HFC-236fa), or 1,1,1,2,3,3,3-heptafluoropropane (HFC-227ea)), hydrocarbon (e.g., propane, cyclopropane, n-butane, isobutane, cyclobutene, n-pentane, 2-methylbutane, cyclopentane, n-hexane, 2-methylpentane, or 2,3-dimethylbutane), or a hydrofluoroolefin (e.g., E-1,3,3,3-tetrafluoro-1-propene (E-HFO-1234ze), or 2,3,3,3-tetrafluoro-1-propene (HFO-1234yf), or a hydrofluoroolefin (e.g., HFO-1225ye isomers, HFO-1234yf isomers, HFO-1234ze isomers, HFO-1336mzz isomers, HCFO-1233zd isomers, or 1,2-dichloro-1,2-difluoroethylene isomers, 2,3,3,3-tetrafluoropropene (HFO-1234yf) (E)-1,3,3,3-tetrafluoroprop-1-ene (E-HFO-1234ze), (Z)-1,3,3,3-tetrafluoroprop-1-ene (Z-HFO-1234ze), or 2-chloro-3,3,3-trifluoropropene (HFO-1243zf), and inert gases (e.g., carbon dioxide, nitrogen or argon), or mixtures thereof.

In some embodiments, the weight ratio of co-propellant component to propellant component is from about 5:95 to about 95:5.

In some embodiments, the present application provides a method of spraying an active material, comprising spraying any of the compositions described herein.

### Foam Blowing Agents

The present application further provides foam blowing agent compositions and foamable compositions. In some embodiments, the foam blowing agent composition or foamable composition comprises one or more of the fluorinated or perfluorinated epoxides provided herein and one or more additional agents. In some embodiments, the foamable composition is preferably a thermoset or thermoplastic foam composition, prepared using the compounds or compositions of the present disclosure. In such foam embodiments, one or more of the present compounds or compositions are included as or part of a blowing agent in a foamable composition, which composition preferably includes one or more additional components capable of reacting and/or foaming under the proper conditions to form a foam or cellular structure. Another aspect relates to foam, and preferably closed cell foam, prepared from a polymer foam formulation containing a blowing agent comprising the compositions of the present disclosure.

Closed-cell polyisocyanate-based foams are widely used for insulation purposes, for example, in building construction and in the manufacture of energy efficient electrical appliances. In the construction industry, polyurethane (polyisocyanurate) board stock is used in roofing and siding for its insulation and load-carrying capabilities. Poured and sprayed polyurethane foams are widely used for a variety of applications including insulating roofs, insulating large structures such as storage tanks, insulating appliances such as refrigerators and freezers, insulating refrigerated trucks and railcars, etc.

A second type of insulating foam is thermoplastic foam, primarily polystyrene foam. Polyolefin foams (e.g., polystyrene, polyethylene, and polypropylene) are widely used in insulation and packaging applications. These thermoplastic foams were generally made with CFC-12 (dichlorodifluoromethane) as the blowing agent. More recently HCFCs (HCFC-22, chlorodifluoromethane) or blends of HCFCs (HCFC-22/HCFC-142b) or HFCs (HFC-152a) have been employed as blowing agents for polystyrene.

A third type of insulating foam is phenolic foam. These foams, which have attractive flammability characteristics, have been generally made with CFC-11 (trichlorofluoromethane) and CFC-113 (1,1,2-trichloro-1,2,2-trifluoroethane) blowing agents.

In addition to closed-cell foams, open-cell foams are also of commercial interest, for example in the production of fluid-absorbent articles. U.S. Pat. No. 6,703,431 (Dietzen, et. al.) describes open-cell foams based on thermoplastics polymers that are useful for fluid-absorbent hygiene articles such as wound contact materials. U.S. Pat. No. 6,071,580 (Bland, et. al.) describes absorbent extruded thermoplastic foams which can be employed in various absorbency applications. Open-cell foams have also found application in evacuated or vacuum panel technologies, for example in the production of evacuated insulation panels as described in U.S. Pat. No. 5,977,271 (Malone). Using open-cell foams in evacuated insulation panels, it has been possible to obtain R-values of 10 to 15 per inch of thickness depending upon the evacuation or vacuum level, polymer type, cell size, density, and open cell content of the foam. These open-cell foams have traditionally been produced employing CFCs, HCFCs, or more recently, HFCs as blowing agents.

Multimodal foams are also of commercial interest, and are described, for example, in U.S. Pat. No. 6,787,580 (Chonde, et. al.) and U.S. Pat. No. 5,332,761 (Paquet, et. al.). A multimodal foam is a foam having a multimodal cell size distribution, and such foams have particular utility in thermally insulating articles since they often have higher insulating values (R-values) than analogous foams having a generally uniform cell size distribution. These foams have been produced employing CFCs, HCFCs, and, more recently, HFCs as the blowing agent.

All of these various types of foams require blowing (expansion) agents for their manufacture. Insulating foams depend on the use of halocarbon blowing agents, not only to foam the polymer, but primarily for their low vapor thermal conductivity, a very important characteristic for insulation value.

The methods of forming a foam generally comprise providing a blowing agent composition of the present disclosure, adding (e.g., directly or indirectly) the blowing agent composition to a foamable composition, and reacting and/or expanding the foamable composition under the conditions effective to form a foam or cellular structure. Any of the methods well known in the art, such as those described in “Polyurethanes Chemistry and Technology,” Volumes I and II, Saunders and Frisch, 1962, John Wiley and Sons, New York, N.Y., which is incorporated herein by reference, may be used or adapted for use in accordance with the foam embodiments.

Accordingly, in some embodiments, the present application provides a composition for use as a foam blowing agent, comprising a blowing agent component which is a compound of Formula (I). In some embodiments, the blowing agent component (i.e., the foam blowing agent) is (Z)-2,3-difluoro-2-(trifluoromethyl)oxirane, (E)-2,3-difluoro-2-(trifluoromethyl)oxirane, trans-2-fluoro-3-(trifluoromethyl)oxirane, trans-2,3-bis(trifluoromethyl)oxirane, trans-2-(trifluoromethyl)-3-(perfluoroethyl)oxirane, or a mixture thereof. In some embodiments, the component is one of the preceding compounds of Formula (I), wherein the compound is free of the opposite stereoisomers.

In some embodiments, the foam blowing agent composition further comprises one or more of methyl formate, dimethoxymethane, E-1,1,1,4,4,4-hexafluoro-2-butene, Z-1,1,1,4,4,4-hexafluoro-2-butene, E-1-chloro-3,3,3-trifluoropropene, Z-1-chloro-3,3,3-trifluoropropene, 1,1,1,2-tetrafluoroethane, 1,1-difluoroethane, dimethyl ether, ethanol, n-propane, n-butane, isobutane, n-pentane, isopentane, cyclopentane, CFO-1112, 1,2-dichloro-1,2-difluoroethylene, carbon dioxide, or water.

In some embodiments, the foam blowing agent composition comprises about 0.1% to 100%, about 0.1% to about 99%, about 1% to about 99%, about 10% to 99%, about 10% to about 99%, about 20% to about 99%, about 30% to about 90%, about 40% to about 99%, about 50% to about 99%, about 60% to about 99%, about 70% to about 99%, about 80% to about 99%, about 90% to about 99%, about 40% to about 90%, about 50% to about 90%, about 60% to about 90%, about 70% to about 90%, about 60% to about 80%, or about 50% to about 70% w/w of the compound of Formula (I) or a mixture of compounds of Formula (I).

The present application further provides a foamable composition for use in formation of a foam, comprising the foam blowing agent composition described herein and one or more additional components capable of reacting and/or foaming under the proper conditions to form a foam or cellular structure.

In some embodiments, the one or more additional components comprises an isocyanate, at least one polyol, and at least one catalyst.

In some embodiments, the one or more additional components comprises a resin, wherein said resin is polystyrene, polypropylene or polyethylene.

In some embodiments, the foamable composition further comprises a nucleating agent, a fire retardant, or a combination thereof.

Representative foamed products that can be made in accordance with the present disclosure include, for example: (1) polystyrene foam sheet for the production of disposable thermoformed packaging materials; e.g., as disclosed in York, U.S. Pat. No. 5,204,169; (2) extruded polystyrene foam boards for use as residential and industrial sheathing and roofing materials, which may be from about 0.5 to 6 inches (1.25 to 15 cm) thick, up to 4 feet (122 cm) wide, with cross-sectional areas of from 0.17 to 3 square feet (0.016 to 0.28 square meter), and up to 27 feet (813 meters) long, with densities of from about 1.5 to 10 pounds per cubic foot (pcf) (25 to 160 kilograms per cubic meter (kg/m³); (3) expandable foams in the form of large billets which may be up to about 2 feet (61 cm) thick, often at least 1.5 feet 46 cm) thick, up to 4 feet (1.22 meters) wide, up to 16 feet (4.8 meters) long, having a cross-sectional area of about 2 to 8 square feet (0.19 to 0.74 square meter) and a density of from 6 to 15 pcf (96 to 240 kg/m³). Such foamed products are more fully described by Stockdopole and Welsh in the Encyclopedia of Polymer Science and Engineering, vol. 16, pages 193-205, John Wiley & Sons, 1989; hereby incorporated by reference.

In certain embodiments, it is often desirable to employ certain other ingredients in preparing foams. Among these additional ingredients are, catalysts, surfactants, flame retardants, preservatives, colorants, antioxidants, reinforcing agents, fillers, antistatic agents, solubilizing agents, IR attenuating agents, nucleating agents, cell controlling agents, extrusion aids, stabilizing agents, thermally insulating agents, plasticizers, viscosity modifiers, impact modifiers, gas barrier resins, polymer modifiers, rheology modifiers, antibacterial agents, vapor pressure modifiers, UV absorbers, cross-linking agents, permeability modifiers, bitterants, propellants, and the like.

Polyurethane foams are generally prepared by combining and reacting an isocyanate with a polyol in the presence of a blowing or expanding agent and auxiliary chemicals added to control and modify both the polyurethane reaction itself and the properties of the final polymer. For processing convenience, these materials can be premixed into two non-reacting parts typically referred to as the “A-side” and the “B-side.”

The term “A-side” is intended to mean isocyanate or isocyanate containing mixture. An isocyanate containing mixture may include the isocyanate, the blowing or expanding agent and auxiliary chemicals, like catalysts, surfactants, stabilizers, chain extenders, cross-linkers, water, fire retardants, smoke suppressants, pigments, coloring materials, fillers, etc.

The term “B-side” is intended to mean polyol or polyol containing mixture. A polyol containing mixture usually includes the polyol, the blowing or expanding agent and auxiliary chemicals, like catalysts, surfactants, stabilizers, chain extenders, cross-linkers, water, fire retardants, smoke suppressants, pigments, coloring materials, fillers, etc.

To prepare the foam, appropriate amounts of A-side and B-side are then combined to react.

When preparing a foam by a process disclosed herein, it is generally preferred to employ a minor amount of a surfactant to stabilize the foaming reaction mixture until it cures. Such surfactants may comprise, for example, a liquid or solid organosilicone compound. Other surfactants include, but are not limited to, polyethylene glycol ethers of long chain alcohols, tertiary amine or alkanolamine salts of long chain alkyl acid sulfate esters, alkyl sulfonic esters and alkyl arylsulfonic acids. The surfactants are employed in amounts sufficient to stabilize the foaming reaction mixture against collapse and to prevent the formation of large, uneven cells. About 0.2 to about 5 parts or even more of the surfactant per 100 parts by weight of polyol are usually sufficient.

One or more catalysts for the reaction of the polyol with the polyisocyanate may also be used. Any suitable urethane catalyst may be used, including but not limited to, tertiary amine compounds and organometallic compounds. Such catalysts are used in an amount which measurably increases the rate of reaction of the polyisocyanate. Typical amounts are about 0.1 to about 5 parts of catalyst per 100 parts by weight of polyol.

Thus, in some embodiments, the invention is directed to a closed cell foam prepared by foaming a foamable composition in the presence of a blowing agent described herein.

Another aspect is for a foam premix composition comprising a polyol and a blowing agent described herein.

Additionally, one aspect is for a method of forming a foam comprising:

(a) adding to a foamable composition a blowing agent described above; and

(b) reacting the foamable composition under conditions effective to form a foam.

In the context of polyurethane foams, the terms “foamable composition” and “foamable component” shall be understood herein to mean isocyanate or an isocyanate-containing mixture. In the context of polystyrene foams, the terms “foamable composition” and “foamable component” shall be understood herein to mean a polyolefin or a polyolefin-containing mixture.

A further aspect is for a method of forming a polyisocyanate-based foam comprising reacting at least one organic polyisocyanate with at least one active hydrogen-containing compound in the presence of a blowing agent described above. Another aspect is for a polyisocyanate foam produced by said method.

Accordingly, the present application provides a process of forming a foam, comprising reacting or extruding a foam blowing composition provided herein under conditions effective to form a foam.

The present application further provides a process of forming a polyisocyanate-based foam, comprising reacting the foam blowing composition provided herein under conditions effective to form a foam, wherein the one or more additional components of the composition comprises at least one organic polyisocyanate and at least one active hydrogen-containing compound.

In some embodiments, the at least one active hydrogen-containing compound is preblended with the blowing agent before reacting with at least one polyisocyanate.

In some embodiments, the at least one active hydrogen-containing compound/blowing agent blend contains at least 2 to about 50 wt. % blowing agent, based on the total weight of active hydrogen-containing compound and blowing agent.

In some embodiments, the at least one active hydrogen-containing compound/blowing agent blend contains at least 2 to about 25 wt. % blowing agent, based on the total weight of active hydrogen-containing compound and blowing agent.

The present application further provides a method of producing a polyurethane foam, comprising reacting the foam blowing composition provided herein under conditions effective to form a foam, wherein the one or more additional components of the composition comprises at least one active hydrogen containing compound, which is mixed with the blowing agent to form a B side mixture, and at least one organic polyisocyanate that forms an A side mixture.

In some embodiments, the at least one organic polyisocyanate, the at least one active hydrogen-containing compound, and the blowing agent component are blended simultaneously.

In some embodiments, the reacting step is performed in the presence of at least one catalyst.

In some embodiments, the B side further comprises at least one auxiliary component, said auxiliary component selected from the group consisting of a surfactant, a flame retardant, a preservative, a colorant, an antioxidant, a reinforcing agent, a filler, an antistatic agent, or a combination thereof.

In some embodiments, the at least surfactant is present in a range of from about 0.2 to about 5 parts surfactant per 100 parts by weight polyol.

In some embodiments, the at least one surfactant is a liquid or solid organosilicone compound, a polyethylene glycol ether of a long chain alcohol, a tertiary amine or alkanolamine salt of a long chain alkyl acid sulfate ester, an alkyl sulfonic ester, or an alkyl arylsulfonic acid.

In some embodiments, the at least one catalyst is present in a range of from about 0.1 to about 5 parts catalyst per 100 parts by weight of polyol.

The present application further provides a process of forming a thermoplastic foam, comprising the steps of: (a) forming a melt comprising a foamable composition, wherein said foamable composition comprises polystyrene, polyethylene, or polypropylene; (b) blending the blowing agent component with the melt to form a composition provided herein at nonfoaming temperatures and pressures as a plasticized melt; (c) passing the plasticized mass at a controlled rate, temperature and pressure through a die and into an expansion zone to form an extrudate; (d) allowing the extrudate to foam in the expansion zone, maintaining the expanding extrudate under such temperatures and pressures for a time sufficient for the viscosity of the extrudate to increase such that the cell size and density of the foam remain substantially unchanged and substantially free of ruptured cells at 25° C. and atmospheric pressure.

In some embodiments, the foamable composition further comprises a nucleating agent, a fire retardant, or a combination thereof.

In some embodiments, the plasticized mass comprises about 80 to about 99 wt. % polystyrene resin, about 2 to about 20 wt. % blowing agent, and about 0.1 to about 5 wt. % nucleating agent.

In some embodiments, the plasticized mass comprises about 80 to about 99 wt. % polypropylene resin, about 2 to about 20 wt. % blowing agent, and about 0.1 to about 5 wt. % nucleating agent.

In some embodiments, the plasticized mass comprises about 80 to about 99 wt. % polyethylene resin, about 2 to about 20 wt. % blowing agent, and about 0.1 to about 5 wt. % nucleating agent.

### Solvents & Cleaning Fluids

The compounds and compositions provided herein may also be used as inert media for polymerization reactions, fluids for removing particulates from metal surfaces, as carrier fluids that may be used, for example, to place a fine film of lubricant on metal parts or as buffing abrasive agents to remove buffing abrasive compounds from polished surfaces such as metal. The compounds and compositions of the invention may also be used as displacement drying agents for removing water, such as from jewelry or metal parts, as resist developers in conventional circuit manufacturing techniques including chlorine-type developing agents, or as strippers for photoresists when used with, for example, a chlorohydrocarbon such as 1,1,1-trichloroethane or trichloroethylene.

In some embodiments, the compounds and compositions provided herein may have utility as novel solvents, carrier fluids, dewatering agents, degreasing solvents or defluxing solvents. It is desirable to identify new agents for these applications with reduced global warming potential.

In some embodiments, the compound or composition provided herein is a compound or composition for use as a solvent. Preferably, the solvent component has a boiling point of 25° C. to 65° C.

In some embodiments, the solvent component is a compound of Formula (I) (i.e., a solvent component). In some embodiments, the solvent component comprises a compound of Formula (I) selected from (Z)-2,3-difluoro-2-(trifluoromethyl)oxirane, (E)-2,3-difluoro-2-(trifluoromethyl)oxirane, cis-2-fluoro-3-(trifluoromethyl)oxirane, trans-2-fluoro-3-(trifluoromethyl)oxirane, trans-2,3-bis(trifluoromethyl)oxirane, cis-2,3-bis(trifluoromethyl)oxirane, trans-2-(trifluoromethyl)-3-(perfluoroethyl)oxirane, trans-2-(trifluoromethyl)-3-(perfluoropropan-2-yl)oxirane, trans-2,3-bis(perfluoropropyl)oxirane, trans-2-(perfluorobutyl)-3-(perfluoroethyl)oxirane, trans-2,3-bis(perfluorobutyl)oxirane, (Z)-2-(2,2,2-Trifluoroethoxy)-3-fluoro-2-(trifluoromethyl)-3-(perfluoroethyl)oxirane, (E)-2-(2,2,2-Trifluoroethoxy)-3-fluoro-2-(trifluoromethyl)-3-(perfluoroethyl)oxirane, cis-2,3-dichloro-2,3-bis(trifluoromethyl)oxirane, trans-2,3-dichloro-2,3-bis(trifluoromethyl)oxirane, trans-2-fluoro-3-(perfluoropropan-2-yl)oxirane, cis-2-fluoro-3-(perfluoropropan-2-yl)oxirane, cis-2,2,3,3,4,4-hexafluoro-6-oxa-bicyclo[3.1.0]hexane, cis-2,2,3,3-tetrafluoro-5-oxabicyclo[2.1.0]pentane, cis-2,3-difluoro-2-(perfluoroethyl)-3-(perfluoropropyl)oxirane, trans-2,3-difluoro-2-(perfluoroethyl)-3-(perfluoropropyl)oxirane, cis-2,3-difluoro-2-(trifluoromethyl)-3-(perfluoropentyl)oxirane, and trans-2,3-difluoro-2-(trifluoromethyl)-3-(perfluoropentyl)oxirane, or a mixture thereof. In some embodiments, the component is one of the preceding compounds of Formula (I), wherein the compound is free of the opposite stereoisomers.

In some embodiments, the solvent composition comprises about 0.1% to 100%, about 0.1% to about 99%, about 1% to about 99%, about 10% to 99%, about 10% to about 99%, about 20% to about 99%, about 30% to about 90%, about 40% to about 99%, about 50% to about 99%, about 60% to about 99%, about 70% to about 99%, about 80% to about 99%, about 90% to about 99%, about 40% to about 90%, about 50% to about 90%, about 60% to about 90%, about 70% to about 90%, about 60% to about 80%, or about 50% to about 70% w/w of the compound of Formula (I) or a mixture of compounds of Formula (I). In some embodiments, the composition consists of the compound of Formula (I), or a mixture of compound of Formula (I).

In some embodiments, the solvent and/or cleaning fluid compositions provided herein may further comprise a propellant. Aerosol propellant may assist in delivering the present composition from a storage container to a surface in the form of an aerosol. Aerosol propellant is optionally included in the present composition in up to about 25 weight percent of the total composition. Representative aerosol propellants may include, but are not limited to, air, nitrogen, carbon dioxide, difluoromethane (CF₂H2, HFC-32), trifluoromethane (CF₃H, HFC-23), difluoroethane (CHF₂CH₃, HFC-152a), trifluoroethane (CH₃CF₃, HFC-143a; or CHF₂CH₂F, HFC-143), tetrafluoroethane (CF₃CH₂F, HFC-134a; or CF₂HCF₂H, HFC-134), pentafluoroethane (CF₃CF₂H, HFC-125), 1,3,3,3-tetrafluoro-1-propene (HFO-1234ze), 2,3,3,3-tetrafluoro-1-propene (HFO-1234yf), 1,2,3,3,3-pentafluoropropene (HFO-1225ye), 1,1,3,3,3-pentafluoropropene (HFO-1225ze) and/or hydrocarbons, such as propane, butanes, or pentanes, or dimethyl ether.

In some embodiments, the solvent and/or cleaning fluid compositions provided herein may further comprise at least one surfactant, including, but not limited to, all surfactants known in the art for dewatering or drying of substrates. Representative surfactants include, for example, alkyl phosphate amine salts (such as a 1:1 salt of 2-ethylhexyl amine and isooctyl phosphate); ethoxylated alcohols, mercaptans or alkylphenols; quaternary ammonium salts of alkyl phosphates (with fluoroalkyl groups on either the ammonium or phosphate groups); and mono- or di-alkyl phosphates of fluorinated amines. Additional fluorinated surfactant compounds are described in U.S. Pat. No. 5,908,822, incorporated herein by reference.

In some embodiments, the compound or composition provided herein is a fluid for removal of particulates from metal surfaces, a carrier fluid, a dewatering agent, a degreasing solvent, or a defluxing solvent.

In some embodiments, the present application provides a process for removing at least a portion of water from, (i.e., dewatering), the surface of a wetted substrate, which comprises contacting the substrate with the aforementioned dewatering composition, and then removing the substrate from contact with the dewatering composition.

The present application further provides a process for removing at least a portion of water from the surface of a wetted substrate, comprising contacting the substrate with the solvent composition provided herein and then removing the substrate from contact with the composition. In some embodiments, the composition further comprises at least one surfactant suitable for dewatering or drying of substrates.

In some embodiments, water originally bound to the surface of the substrate is displaced by solvent and/or surfactant and leaves with the dewatering composition. By “at least a portion of water” is meant at least about 75 weight percent of water at the surface of a substrate is removed per immersion cycle. By “immersion cycle” is meant one cycle involving at least a step wherein substrate is immersed in the present dewatering composition. Optionally, minimal amounts of surfactant remaining adhered to the substrate can be further removed by contacting the substrate with surfactant-free halocarbon solvent. Holding the article in the solvent vapor or refluxing solvent will further decrease the presence of surfactant remaining on the substrate. Removal of solvent adhering to the surface of the substrate is effected by evaporation. Evaporation of solvent at atmospheric or subatmospheric pressures can be employed and temperatures above and below the boiling point of the halocarbon solvent can be used.

Many industries use aqueous compositions for the surface treatment of metals, ceramics, glasses, and plastics. Cleaning, plating, and deposition of coatings are often carried out in aqueous media and are usually followed by a step in which residual water is removed. Hot air drying, centrifugal drying, and solvent-based water displacement are methods used to remove such residual water.

While hydrofluorocarbons (HFCs) have been proposed as replacements for the previously used CFC solvents in drying or dewatering applications, many HFCs have limited solvency for water. The use of surfactant, which assists in removal of water from substrates is therefore necessary in many drying or dewatering methods. Hydrophobic surfactants have been added to dewatering or drying solvents to displace water from substrates.

The primary function of the dewatering or drying solvent (e.g., compounds or compositions provided herein) in a dewatering or drying composition is to reduce the amount of water on the surface of a substrate being dried. The primary function of the surfactant is to displace any remaining water from the surface of the substrate. When the composition and surfactant are combined, a highly effective displacement drying composition is attained.

The amount of surfactant included in a dewatering composition of the present invention can vary widely depending on the particular drying application in which said composition will be used, but is readily apparent to those skilled in the art. In some embodiments, the amount of surfactant is at least about 50 parts per million (ppm, on a weight basis). In some embodiments, the amount of surfactant is from about 100 to about 5000 ppm. In some embodiments, the amount of surfactant used is from about 200 to about 2000 ppm based on the total weight of the dewatering composition.

Optionally, other additives may be included in the compositions for use as dewatering compositions. Such additives include compounds having antistatic properties; the ability to dissipate static charge from non-conductive substrates such as glass and silica. Use of an antistatic additive in the dewatering compositions of the present invention may be necessary to prevent spots and stains when drying water or aqueous solutions from electrically non-conductive parts such as glass lenses and mirrors.

The fluorinated and perfluorinated compounds of the present invention may also have utility as dielectric fluids, i.e., they are poor conductors of electric current and do not easily dissipate static charge. Boiling and general circulation of dewatering compositions in conventional drying and cleaning equipment can create static charge, particularly in the latter stages of the drying process where most of the water has been removed from a substrate.

Such static charge collects on non-conductive surfaces of the substrate and prevents the release of water from the surface. The residual water dries in place resulting in undesirable spots and stains on the substrate. Static charge remaining on substrates can bring out impurities from the cleaning process or can attract impurities such as lint from the air, which results in unacceptable cleaning performance.

In some embodiments, desirable antistatic additives are polar compounds, which are soluble in the solvent compositions of the invention and result in an increase in the conductivity solvent composition resulting in dissipation of static charge from a substrate. In some embodiments, the antistatic additives have a normal boiling point near that of the solvent composition and have minimal to no solubility in water. In some embodiments, the antistatic additives have a solubility in water of less than about 0.5 weight percent. In some embodiments, the solubility of antistatic agent is at least 0.5 weight percent in a solvent composition provided herein. In some embodiments, the antistatic additive is nitromethane (CH₃NO₂).

In some embodiments, the dewatering or drying method of the present disclosure is effective in displacing water from a broad range of substrates including metals, such as tungsten, copper, gold, beryllium, stainless steel, aluminum alloys, brass, and the like; from glasses and ceramic surfaces, such as glass, sapphire, borosilicate glass, alumina, silica such as silicon wafers used in electronic circuits, fired alumina and the like; and from plastics such as polyolefin (“Alathon”, Rynite®, “Tenite”), polyvinylchloride, polystyrene (Styron), polytetrafluoroethylene (Teflon®), tetrafluoroethylene-ethylene copolymers (Tefzel®), polyvinylidenefluoride (“Kynar”), ionomers (Surlyn®), acrylonitrile-butadiene-styrene polymers (Kralac®), phenol-formaldehyde copolymers, cellulosic (“Ethocel”), epoxy resins, polyacetal (Delrin®), poly(p-phenylene oxide) (Noryl®), polyetherketone (“Ultrapek”), polyetheretherketone (“Victrex”), poly(butylene terephthalate) (“Valox”), polyarylate (Arylon®), liquid crystal polymer, polyimide (Vespel®), polyetherimides (“Ultem”), polyamideimides (“Torlon”), poly(p-phenylene sulfide) (“Rython”), polysulfone (“Udel”), and polyaryl sulfone (“Rydel”). In some embodiments, the compositions for use in the present dewatering or drying methods are compatible with elastomers.

Methods of contacting the substrate with dewatering composition are not critical and can vary widely. For example, the substrate can be immersed in the composition, or the substrate can be sprayed with the composition using conventional equipment. Complete immersion of the substrate is preferred as it generally insures contact between the composition and all exposed surfaces of the substrate. However, any other method, which can easily provide such complete contact may be used.

The time period over which substrate and dewatering composition are contacted can vary widely. Usually, the contacting time is up to about 5 minutes, however, longer times may be used if desired. In one embodiment of the dewatering process, the contacting time is from about 1 second to about 5 minutes. In another embodiment, the contacting time of the dewatering process is from about 15 seconds to about 4 minutes.

Contacting temperatures can also vary widely depending on the boiling point of the composition. In general, the contacting temperature is equal to or less than the composition's normal boiling point.

In some embodiments, the solvent compositions of the present disclosure may further contain a co-solvent. Such co-solvents are desirable where the present compositions are employed in cleaning conventional process residue from substrates, e.g., removing soldering fluxes and degreasing mechanical components comprising substrates of the present invention. Such co-solvents include, but are not limited to, alcohols (such as methanol, ethanol, isopropanol), ethers (such as diethyl ether, methyl tertiary-butyl ether), ketones (such as acetone), esters (such as ethyl acetate, methyl dodecanoate, isopropyl myristate and the dimethyl or diisobutyl esters of succinic, glutaric or adipic acids or mixtures thereof), ether alcohols (such as propylene glycol monopropyl ether, dipropylene glycol monobutyl ether, and tripropylene glycol monomethyl ether), and hydrocarbons (such as pentane, cyclopentane, hexane, cyclohexane, heptane, octane), and hydrochlorocarbons (such as trans-1,2-dichloroethylene). In some embodiments, the solvent composition further comprises trans-1,2-dichloroethylene, cyclopentane, cyclohexane, methyl acetate, acetone, methyl formate, ethyl formate, ethyl acetate, heptane, methanol, ethanol, isopropyl alcohol, dimethyl carbonate, propylene carbonate, tertiary butyl acetate, or methyl ethyl ketone. When such a co-solvent is employed with the present composition for substrate dewatering or cleaning, it may be present in an amount of from about 1 weight percent to about 95 weight percent based on the weight of the overall composition. In some embodiments, when such a co-solvent is employed with the present composition for substrate dewatering or cleaning, it may be present in an amount of from about 1 weight percent to about 50 weight percent based on the weight of the overall composition.

For proper operation in use, microelectronic components must be cleaned of flux residues, oils and greases, and particulates that may contaminate the surfaces after completion of manufacture. In some embodiments, the present disclosure provides a process for removing residue from a surface or substrate comprising contacting the surface or substrate with a cleaning composition or cleaning agent of the present invention and, optionally, recovering the surface or substrate substantially free of residue from the cleaning composition or cleaning agent.

In some embodiments, the present disclosure provides a method for cleaning surfaces by removing contaminants from the surface. The method for removing contaminants from a surface comprises contacting the surface having contaminants with a cleaning composition of the present invention to solubilize the contaminants and, optionally, recovering the surface from the cleaning composition. The surface is then substantially free of contaminants. In some embodiments, the contaminants or residues that may be removed by the present method include, but are not limited to oils and greases, flux residues, and particulate contaminants.

In some embodiments, the contacting may be accomplished by spraying, flushing, or wiping with a substrate (e.g., wiping cloth or paper, that has the cleaning composition incorporated in or on it). In another embodiment of the method, the contacting may be accomplished by dipping or immersing the surface in a bath of the cleaning composition.

In some embodiments, the recovering is performed by removing the surface that has been contacted from the cleaning composition bath (e.g., in a similar manner as described for the method for depositing an a fluorolubricant on a surface as described below). In some embodiments, the recovering is performed by allowing the cleaning composition that has been sprayed, flushed, or wiped to drain away. Additionally, any residual cleaning composition that may be left behind after the completion of the previous steps may be evaporated in a manner similar to that for the deposition method as well.

The method for cleaning a surface may be applied to the same types of surfaces as the method for deposition as described below. For example, semiconductor surfaces or magnetic media disks of silica, glass, metal or metal oxide, or carbon may have contaminants removed by the method. In the methods described herein, contaminant may be removed from a disk by contacting the disk with the cleaning composition and recovering the disk from the cleaning composition.

In some embodiments, the present method also provides methods of removing contaminants from a product, part, component, substrate, or any other article or portion thereof by contacting the article with a cleaning composition of the present invention. For the purposes of convenience, the term “article” is used herein to refer to all such products, parts, components, substrates, and the like and is further intended to refer to any surface or portion thereof. Furthermore, the term “contaminant” is intended to refer to any unwanted material or substance present on the article, even if such substance is placed on the article intentionally. For example, in the manufacture of semiconductor devices it is common to deposit a photoresist material onto a substrate to form a mask for the etching operation and to subsequently remove the photoresist material from the substrate. The term “contaminant” as used herein is intended to cover and encompass such a photo resist material. Hydrocarbon based oils and greases and dioctylphthalate are examples of the contaminants that may be found on the carbon coated disks.

In some embodiments, the method comprises contacting the article with a cleaning composition of the invention, in a vapor degreasing and solvent cleaning method. In some embodiments, vapor degreasing and solvent cleaning methods consist of exposing an article, preferably at room temperature, to the vapors of a boiling cleaning composition. Vapors condensing on the object have the advantage of providing a relatively clean, distilled cleaning composition to wash away grease or other contamination. Such processes thus have an additional advantage in that final evaporation of the present cleaning composition from the object leaves behind relatively little residue as compared to situations where the object is washed in liquid cleaning composition.

In some embodiments, for applications in which the article includes contaminants that are difficult to remove, the present methods involve raising the temperature of the cleaning composition above ambient or to any other temperature that is effective in such application to substantially improve the cleaning action of the cleaning composition. In some embodiments, such processes are also generally used for large volume assembly line operations where the cleaning of the article, particularly metal parts and assemblies, must be performed efficiently and quickly.

In some embodiments, the cleaning methods of the present invention comprise immersing the article to be cleaned in liquid cleaning composition at an elevated temperature. In some embodiments, the cleaning methods of the present invention comprise immersing the article to be cleaned in liquid cleaning composition at about the boiling point of the cleaning composition. In some embodiments, this step removes a substantial amount of the target contaminant from the article (e.g., removing from about 95% to about 99% of the target contaminant from the article). In some embodiments, this step removes a major portion of the target contaminant from the article (e.g., removing greater than about 50% of the target contaminant from the article). In one embodiment, this step is then followed by immersing the article in freshly distilled cleaning composition, which is at a temperature below the temperature of the liquid cleaning composition in the preceding immersion step. In some embodiments, the freshly distilled cleaning composition is at about ambient or room temperature. In some embodiments, the method further comprises the step of contacting the article with relatively hot vapor of the cleaning composition, by exposing the article to vapors rising from the hot/boiling cleaning composition associated with the first mentioned immersion step. In some embodiments, this results in condensation of the cleaning composition vapor on the article. In some embodiments, the article may be sprayed with distilled cleaning composition before a final rinsing.

It is contemplated that numerous varieties and types of vapor degreasing equipment are adaptable for use in connection with the present methods. One example of such equipment and its operation is disclosed by U.S. Pat. No. 3,085,918, which is incorporated herein by reference. The equipment disclosed therein includes a boiling sump for containing a cleaning composition, a clean sump for containing distilled cleaning composition, a water separator, and other ancillary equipment.

The present cleaning methods may also comprise cold cleaning in which the contaminated article is either immersed in the fluid cleaning composition of the present invention under ambient or room temperature conditions or wiped under such conditions with rags or similar objects soaked in the cleaning composition.

Accordingly, the present application further provides a process for dissolving a solute, comprising contacting and mixing said solute with a sufficient quantity of a solvent composition provided herein.

The present application further provides a process of cleaning a surface, comprising contacting a solvent composition provided herein with said surface.

In some embodiments, the compounds and compositions provided herein may be useful as solvents in fluorolubricant compositions. Fluorolubricants are widely used as lubricants in the magnetic disk drive industry to decrease the friction between the head and disk, that is, reduce the wear and therefore minimize the possibility of disk failure. Invariably, during normal disk drive application, the head and the disk surface will make contact. To reduce wear on the disk, from both sliding and flying contacts, it must be lubricated.

There is a need in the industry for improved methods for deposition of fluorolubricants. The use of certain solvents, such as CFC-113 and PFC-5060, has been regulated due to their impact on the environment. Therefore, solvents that will be used in this application should consider environmental impact. Also, such solvent must dissolve the fluorolubricant and form a substantially uniform or uniform coating of fluorolubricant. Additionally, existing solvents have been found to require higher fluorolubricant concentrations to produce a given thickness coating and produce irregularities in uniformity of the fluorolubricant coating.

Accordingly, the present application further provides a process for depositing a coating on a surface, comprising contacting the solvent composition provided herein with said surface, wherein the composition further comprises a depositable material. In some embodiments, the depositable material comprises a fluorolubricant or a photoresist.

In some embodiments, the fluorolubricants of the present disclosure comprise perfluoropolyether (PFPE) compounds, or lubricant comprising X-1P®, which is a phosphazene-containing disk lubricant. These perfluoropolyether compounds are sometimes referred to as perfluoroalkylethers (PFAE) or perfluoropolyalkylethers (PFPAE). These PFPE compounds range from simple perfluorinated ether polymers to functionalized perfluorinated ether polymers. PFPE compounds of different varieties that may be useful as fluorolubricant in the present invention are available from several sources. In another embodiment, useful fluorolubricants for the present inventive method include but are not limited to Krytox® GLP 100, GLP 105 or GLP 160 (E. I. du Pont de Nemours & Co., Fluoroproducts, Wilmington, Del., 19898, USA); Fomblin® Z-Dol 2000, 2500 or 4000, Z-Tetraol, or Fomblin® AM 2001 or AM 3001 (sold by Solvay Solexis S.p.A., Milan, Italy); Demnum™ LR-200 or S-65 (offered by Daikin America, Inc., Osaka, Japan); X-1P® (a partially fluorinated hyxaphenoxy cyclotriphosphazene disk lubricant available from Quixtor Technologies Corporation, a subsidiary of Dow Chemical Co, Midland, Mich.); and mixtures thereof. The Krytox® lubricants are perfluoroalkylpolyethers having the general structure F(CF(CF₃)CF₂O)ₙ—CF₂CF₃, wherein n ranges from 10 to 60. The Fomblin® lubricants are functionalized perfluoropolyethers that range in molecular weight from 500 to 4000 atomic mass units and have general formula X—CF₂—O(CF₂—CF₂—O)ₚ—(CF₂O)_{q}—CF₂—X, wherein X may be —CH₂OH, CH₂(O—CH₂—CH₂)ₙOH, CH₂OCH₂CH(OH)CH₂OH or —CH₂O—CH₂-piperonyl. The Demnum™ oils are perfluoropolyether-based oils ranging in molecular weight from 2700 to 8400 atomic mass units. Additionally, new lubricants are being developed such as those from Moresco (Thailand) Co., Ltd, which may be useful in the present inventive method.

The fluorolubricant compositions of the present invention may further comprise Z-DPA (Hitachi Global Storage Technologies, San Jose, Calif.), a PFPE terminated with dialkylamine end-groups. The nucleophilic end-groups serve the same purpose as X1P®, thus providing the same stability without any additive.

The surface on which the fluorolubricant may be deposited is any solid surface that may benefit from lubrication. Semiconductor materials such as silica disks, metal or metal oxide surfaces, vapor deposited carbon surfaces or glass surfaces are representative of the types of surfaces for which the methods of the present invention are useful. The present inventive method is particularly useful in coating magnetic media such as computer drive hard disks. In the manufacture of computer disks, the surface may be a glass, or aluminum substrate with layers of magnetic media that is also coated by vapor deposition with a thin (10-50 Angstrom) layer of amorphous hydrogenated or nitrogenated carbon. The fluorolubricant may be deposited on the surface disk indirectly by applying the fluorolubricant to the carbon layer of the disk.

The first step of combining the fluorolubricant and solvent may be accomplished in any suitable manner such as mixing in a suitable container such as a beaker or other container that may be used as a bath for the deposition method. The fluorolubricant concentration in the solvent provided herein may be from about 0.010 percent (wt/wt) to about 20 percent (wt/wt).

The step of contacting said composition comprising fluorolubricant and solvent with the surface may be accomplished in any manner appropriate for said surface (considering the size and shape of the surface). A hard drive disk must be supported in some manner such as with a mandrel or some other support that may fit through the hole in the center of the disk. The disk will thus be held vertically such that the plane of the disk is perpendicular to the solvent bath. The mandrel may have different shapes including but not limited to, a cylindrical bar, or a V-shaped bar. The mandrel shape will determine the area of contact with the disk. The mandrel may be constructed of any material strong enough to hold the disk, including but not limited to metal, metal alloy, plastic or glass. Additionally, a disk may be supported vertically upright in a woven basket or be clamped into a vertical position with 1 or more clamps on the outer edge. The support may be constructed of any material with the strength to hold the disk, such as metal, metal alloy, plastic or glass. However the disk is supported, the disk will be lowered into a container holding a bath of the fluorolubricant/solvent combination. The bath may be held at room temperature or be heated or cooled to temperatures ranging from about 0° C. to about 50° C.

Alternatively, the disk may be supported as described above and the bath may be raised to immerse the disk. In either case, the disk may then be removed from the bath (either by lowering the bath or by raising the disk). Excess fluorolubricant/solvent compositions can be drained into the bath.

When dip coating is used for depositing fluorolubricant on the surface, the pulling-up speed (speed at which the disk is removed from the bath), and the density of the fluorolubricant and the surface tension are relevant for determining the resulting film thickness of the fluorolubricant. Awareness of these parameters for obtaining the desired film thickness is required. Details on how these parameters effect coatings are given in, “Dip-Coating of Ultra-Thin Liquid Lubricant and its Control for Thin-Film Magnetic Hard Disks” in IEEE Transactions on Magnetics, vol. 31, no. 6, November 1995.

### Gaseous Dielectrics

A dielectric gas, or insulating gas, is a dielectric material in gaseous state. Its main purpose is to prevent or rapidly quench electric discharges. Dielectric gases are used as electrical insulators in high voltage applications, e.g., transformers, circuit breakers, switchgear (namely high voltage switchgear), and radar waveguides. As used herein, the term “high voltage” shall be understood to mean above 1000 V for alternating current, and at least 1500 V for direct current. The inventive compositions can be useful as gaseous dielectrics in high voltage applications.

Accordingly, in some embodiments, the present application provides a composition is for use in preventing or rapidly quenching an electric discharge, wherein the composition comprises a dielectric component, which is a compound of Formula (I) as described herein.

Preferably, the gaseous dielectric has a boiling point of −70° C. to 40° C.

In some embodiments, the dielectric component is a compound of Formula (I) selected from (Z)-2,3-difluoro-2-(trifluoromethyl)oxirane, (E)-2,3-difluoro-2-(trifluoromethyl)oxirane, trans-2-fluoro-3-(trifluoromethyl)oxirane, and trans-2,3-bis(trifluoromethyl)oxirane, or a mixture thereof.

In another embodiment, the present application provides a method for preventing or rapidly quenching an electric discharge in a space in a high voltage device comprising injecting a gaseous dielectric into said space, wherein said gaseous dielectric comprises the composition comprising a compound of Formula (I) as described herein. In some embodiments, the composition comprises a compound of Formula (I) selected from (Z)-2,3-difluoro-2-(trifluoromethyl)oxirane, (E)-2,3-difluoro-2-(trifluoromethyl)oxirane, trans-2-fluoro-3-(trifluoromethyl)oxirane, and trans-2,3-bis(trifluoromethyl)oxirane, or a mixture thereof.

### Inhalation Anesthetics

The compounds provided herein (e.g., compounds of Formula (I)) may be useful as anesthetic agents for anesthetizing a subject. As used herein the term “anesthetize’ means to induce a loss of sensation and usually of consciousness without loss of vital functions artificially produced by the administration of one or more agents that block the passage of pain impulses along nerve pathways of the brain.

Accordingly, the present application provides a compound for use as an anesthetic agent, wherein the compound is a compound of Formula (I) described herein.

In some embodiments, the compound of Formula (I) is administered in the form of a composition (e.g., a pharmaceutical composition). In some embodiments, the composition further comprises oxygen. In some embodiments, the composition further comprises air.

The therapeutic dosage of a compound of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics, and the route of administration. The dosage is likely to depend on such variables as the overall health status of the particular patient, the relative biological efficacy of the compound selected, and formulation of the excipient. Effective doses can be extrapolated from dose-response curves derived from in vitro or animal model test systems.

In some embodiments, the compound or composition is administered by inhalation. For example, the compound or composition may be breathed directly, utilizing a face mask, tent, or intermittent positive pressure breathing machine. In some embodiments, the compound or composition is administered by the nasal respiratory route.

In some embodiments, the compound or composition is administered to the subject in an amount effective to anesthetize the subject. In some embodiments, the effective amount comprises from about 0.1% v/v to about 1.5% v/v of the compound of Formula (I), for example, about 0.1% v/v, about 0.2% v/v, about 0.5% v/v, about 0.75% v/v, about 1% v/v, about 1.2% v/v, or about 1.5% v/v. In some embodiments, the effective amount is about 1% v/v.

In some embodiments, the compound or composition is administered during a surgical procedure. In some embodiments, the compound or composition is administered prior to a surgical procedure.

In some embodiments, administration of the compound or composition to a subject reduces the response to an alerting stimuli of the subject compared to the normal response to an alerting stimuli of the subject. In some embodiments, administration of the compound or composition to the subject induces a loss of consciousness of the subject.

In some embodiments, the compound of Formula (I) is selected from the group consisting of:
2,3-difluoro-2-(trifluoromethyl)oxirane;
2-fluoro-3-(trifluoromethyl)oxirane;
2,3-bis(trifluoromethyl)oxirane;
2-(trifluoromethyl)-3-(perfluoroethyl)oxirane;
2-(trifluoromethyl)-3-(perfluoropropan-2-yl)oxirane;
2,3-bis(perfluoropropyl)oxirane;
2-(perfluorobutyl)-3-(perfluoroethyl)oxirane;
2,3-bis(perfluorobutyl)oxirane;
2-(2,2,2-Trifluoroethoxy)-3-fluoro-2-(trifluoromethyl)-3-(perfluoroethyl)oxirane;
2,3-dichloro-2,3-bis(trifluoromethyl)oxirane;
2-fluoro-3-(perfluoropropan-2-yl)oxirane;
2,2,3,3,4,4-hexafluoro-6-oxa-bicyclo[3.1.0]hexane;
2,2,3,3-tetrafluoro-5-oxabicyclo[2.1.0]pentane;
2,3-difluoro-2-(perfluoroethyl)-3-(perfluoropropyl)oxirane; and
2,3-difluoro-2-(trifluoromethyl)-3-(perfluoropentyl)oxirane;
or a mixture thereof.

In some embodiments, the compound of Formula (I) is selected from the group consisting of:
(Z)-2,3-difluoro-2-(trifluoromethyl)oxirane;
(E)-2,3-difluoro-2-(trifluoromethyl)oxirane;
cis-2-fluoro-3-(trifluoromethyl)oxirane;
trans-2-fluoro-3-(trifluoromethyl)oxirane;
trans-2,3-bis(trifluoromethyl)oxirane;
cis-2,3-bis(trifluoromethyl)oxirane;
trans-2-(trifluoromethyl)-3-(perfluoroethyl)oxirane;
trans-2-(trifluoromethyl)-3-(perfluoropropan-2-yl)oxirane;
trans-2,3-bis(perfluoropropyl)oxirane;
trans-2-(perfluorobutyl)-3-(perfluoroethyl)oxirane;
trans-2,3-bis(perfluorobutyl)oxirane;
(Z)-2-(2,2,2-Trifluoroethoxy)-3-fluoro-2-(trifluoromethyl)-3-(perfluoroethyl)oxirane;
(E)-2-(2,2,2-Trifluoroethoxy)-3-fluoro-2-(trifluoromethyl)-3-(perfluoroethyl)oxirane;
cis-2,3-dichloro-2,3-bis(trifluoromethyl)oxirane;
trans-2,3-dichloro-2,3-bis(trifluoromethyl)oxirane;
trans-2-fluoro-3-(perfluoropropan-2-yl)oxirane;
cis-2-fluoro-3-(perfluoropropan-2-yl)oxirane;
cis-2,2,3,3,4,4-hexafluoro-6-oxa-bicyclo[3.1.0]hexane;
cis-2,2,3,3-tetrafluoro-5-oxabicyclo[2.1.0]pentane;
cis-2,3-difluoro-2-(perfluoroethyl)-3-(perfluoropropyl)oxirane;
trans-2,3-difluoro-2-(perfluoroethyl)-3-(perfluoropropyl)oxirane;
cis-2,3-difluoro-2-(trifluoromethyl)-3-(perfluoropentyl)oxirane; and
trans-2,3-difluoro-2-(trifluoromethyl)-3-(perfluoropentyl)oxirane;
or a mixture thereof.

In some embodiments, the compound of Formula (I) is cis-2,3-bis(trifluoromethyl)oxirane (e.g., (2R,3S)-2,3-bis(trifluoromethyl)oxirane).

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

¹H, ¹³C, and ¹⁹F NMR spectra were recorded in CDCl₃ on a Varian VNMRS (499.87 MHz) instrument using CFCl₃ or TMS as internal reference standards. GC and GC/MS analyses were carried out on an HP-6890 instrument, using an HP FFAP capillary column and either TCD (GC) or mass-selective (GS/MS) detectors, respectively. Acetonitrile, tetrabutylammonium hydrogen sulfate, tetrabutylphosphium bromide Aliquat® 336, xylene, o-xylene were obtained from commercial source (Aldrich) and used without further purification. Commercially available sodium hypochlorite solution (typically 10-15% of available chlorine) was available from Sigma-Aldrich and was stored refrigerated. Purity of all isolated compounds was established to be at least 98-99% by GC and NMR spectroscopy (the remainder was determined to be remaining starting material or solvent unless specified otherwise. Epoxide of perfluoropentene-2 was identified based on reported NMR data and data of GC/MS (see e.g., Kolenko et al, Izv. Akad. Nauk. SSSR, Ser. Khim, 1979, 2509-2512).

### Example 1. Cis-2-fluoro-3-(trifluoromethyl)oxirane

[chemical structure]

A 250 mL round bottom flask was equipped with a magnetic stir bar, a dry ice condenser, and a thermowell. The flask was charged with xylenes (30 mL, 0.24 moles), 15% w/w chilled sodium hypochlorite (100 mL, 1.49 moles), and Aliquat® 336 (5% mol, 1 mL). The reaction mixture was stirred at room temperature. An addition funnel containing olefin (Z)-1,3,3,3-tetrafluoroprop-1-ene, (87.71 mmoles, 10 grams) was fixed onto the reaction flask and olefin was added dropwise to the reaction mixture over a period of 30 minutes. A slight exotherm was observed once the internal temperature reached 25° C. The reaction mixture was sampled every hour over the first 6 hours and then stirred overnight. The contents of the flask were then transferred to a separatory funnel and the aqueous layer was discarded. The organic layer was dried with magnesium sulfate and then filtered into a clean round bottom flask. The filtrate was then subjected to reduced pressure; any remaining olefin and epoxide were collected in a dry ice cold trap and subsequently distilled to provide the desired product with b.p. 54-55° C.; isolated yield: 35%. ¹⁹F (CDCl₃): −68.88 (3F, dm, 12.5 Hz), −164.84 (1F, dqm, 84.0, 12.5, 3.5 Hz) ppm; ¹H NMR (CDCl₃): 3.28 (1H, m, 4.9, 3.0, 1.6 Hz), 5.61 (1H, d quint. 84.0, 1.8 Hz) ppm; ¹³C NMR {H}, (CDCl₃): 57.36 (qd, 42.6, 15.9 Hz), 85.56 (dq, 278.4, 1.5 Hz), 121.42 (qd, 275.4, 6.4 Hz) ppm. MS (m/z): 130 (M⁺, C₃H₂F₄O⁺).

### Example 2. Trans-2-fluoro-3-(trifluoromethyl)oxirane

[chemical structure]

A 5000 mL round bottom flask was equipped with a mechanical stir bar, a dry ice condenser, an addition funnel, and a thermowell. The flask was charged with xylenes (800 mL), 15% w/w chilled sodium hypochlorite (2400 mL), and tetrabutylphosphonium bromide (10% mol, 110 g). The flask was chilled to 0° C. An addition funnel containing olefin (E)-1,3,3,3-tetrafluoroprop-1-ene (3.36 moles, 383 grams) was fixed onto the reaction flask; olefin was added dropwise to the reaction mixture over a period of 60 minutes. An exotherm was initially observed but the controlled by an ice bath to maintain the internal temperature at no more than 15° C. The reaction mixture was sampled every hour over the first 3 hours and then stopped after this time. Once the reaction was complete, the contents of the flask were transferred to a separatory funnel and the aqueous layer was discarded. The organic layer was dried with magnesium sulfate and then filtered into a clean round bottom flask. The filtrate was then subjected to reduced pressure; any remaining olefin and epoxide were collected in a dry ice cold trap and subsequently distilled to provide the desired product with b.p. 19-20° C.; isolated yield: 37%. ¹⁹F NMR (CDCl₃): −73.19 (3F, d, 5.5 Hz), −155.92 (1F, dm, 83.9, 1.4 Hz) ppm; ¹H NMR (CDCl3): 3.67 (1H, dq, 5.1, 2.8 Hz), 5.68 (1H, d, 83.9 Hz) ppm; MS (m/z): 130 (M⁺, C₃H₂F₄O⁺).

### Example 3. Trans-2,3-bis(trifluoromethyl)oxirane

[chemical structure]

A 5000 mL round bottom flask was equipped with a mechanical stir bar, a dry ice condenser, an addition funnel, and a thermowell. The flask was charged with xylenes (800 mL), 15% w/w chilled sodium hypochlorite (2400 mL), and tetrabutylphosphonium bromide (7.5% mol, 85.3 g). The flask was chilled to 0° C. An addition funnel containing olefin (E)-1,1,1,4,4,4-hexafluorobut-2-ene (3.35 moles, 550 grams) was fixed onto the reaction flask; olefin was added dropwise to the reaction mixture over a period of 30 minutes. An exotherm was initially observed but the controlled by an ice bath to maintain the internal temperature at no more than 15° C. The reaction mixture was sampled every hour over the first 3 hours and then stopped after this time. Once the reaction was completed, the contents of the flask were transferred to a separatory funnel the organic layer was separated, dried over magnesium sulfate and filtered into a clean round bottom flask. The crude product was transferred into a dry ice cold trap under reduced pressure and subsequently distilled, to provide the desired product with b.p. 35° C.; isolated yield 80%. NMR data (CDCl₃): ¹⁹F: −74.38 (d, J=3.9 Hz) ppm; ¹H: 3.72 (m, not first order) ppm; ¹³C {H}: 50.38 (qq, J=43.15, 3.12 Hz), 120.91 (q, 275.8) ppm; M/Z: 180 M+ (C₄H₂F₆O).

### Example 4. Cis-2,3-bis(trifluoromethyl)oxirane

[chemical structure]

A 5000 mL round bottom flask was equipped with a mechanical stir bar, a dry ice condenser, an addition funnel, and a thermowell. The flask was charged with xylenes (800 mL), 15% w/w chilled sodium hypochlorite (2400 mL), and tetrabutylphosphonium bromide (7.5% mol, 85.3 g). The flask was chilled to 0° C. An addition funnel containing olefin (Z)-1,1,1,4,4,4-hexafluorobut-2-ene (3.35 moles, 550 grams) was fixed onto the reaction flask; olefin was added dropwise to the reaction mixture over a period of 30 minutes. An exotherm was initially observed but the controlled by an ice bath to maintain the internal temperature at no more than 15° C. The reaction mixture was sampled every hour over the first 3 hours and then stopped after this time. Once the reaction was completed, the contents of the flask were transferred to a separatory funnel the organic layer was separated, dried over magnesium sulfate and filtered into a clean round bottom flask. The crude product was transferred into in a dry ice cold trap under reduced pressure and subsequently distilled, to provide the desired product with b.p. 64-65° C.; 425.8 g; isolated yield 70.5%. NMR data (CDCl₃): ¹⁹F: −67.69 (m) ppm; ¹H: 3.62 (m, not first order) ppm; ¹³C {H}: 52.71 (m), 120.08 (q, 276.2) ppm; MS (m/Z): 180 M⁺ (C₄H₂F₆O).

### Example 5. Trans-2-(trifluoromethyl)-3-(perfluoroethyl)oxirane

[chemical structure]

To a mixture of 40 mL of NaOCl, 10 mL of ACN, and 0.68 g of tetrabutylammonium hydrogensulfate was added 12 g of (E)-1,1,1,4,4,5,5,5-octafluoropent-2-ene and the reaction mixture was vigorously agitated at ambient temperature. The conversion of the olefin was 90% after 24 h. At this point the reaction mixture was diluted with water, and the organic layer was separated, dried over MgSO₄, and distilled to give 7 g (54%) of epoxide, >99% purity, b.p. 49-49.5° C. ¹⁹F NMR (CDCl₃): −74.10 (3F, d, 4.3 Hz), −84.10 (3F, s), −127.03 (1F, ddq, 275.7, 8.8, 1.4 Hz), −127.48 (1F, dd, 275.7, 9.8) ppm; ¹H NMR (CDCl₃): 3.75 (m) ppm; ¹³C {H}NMR (CDCl₃): 49.29 (q, 28.3, 3.0 Hz), 49.75 (qdd, 43.2, 5.5, 3.4 Hz), 10.87 (tq, 256.4, 38.4), 118.13 (qt, 287.6, 53.0 Hz), 120.74 (q, 278.6) ppm.

### Example 6. Trans-2-(trifluoromethyl)-3-(perfluoropropan-2-yl)oxirane

[chemical structure]

To a mixture of 200 mL of NaOCl, 30 mL of toluene, and 3.6 g of Aliquat® 336, 26.4 g (0.1 mol) of (E)-1,1,1,5,5,5-hexafluoro-4,4-bis(trifluoromethyl)pent-2-ene was added dropwise, and the internal temperature was maintained at 20-26° C. The reaction mixture was vigorously agitated for 20 h at ambient temperature at which time conversion of the olefin was determined to be >98%. The reaction mixture was transferred under dynamic vacuum (˜10 mm Hg) into a cold trap (−78° C.), dried over MgSO₄, and distilled using a Vigroux column. Material with b.p. 70-71° C. (4.5 g) was isolated, which was found to be the epoxide containing 5% toluene, (NMR) and an additional 9 g of the epoxide (b.p. 72-90° C.) containing ˜20% toluene. Calculated yield of epoxide was 38%.

### Example 7. Trans-2,3-bis(perfluoropropyl)oxirane

[chemical structure]

The title compound was prepared following the same protocol described in Example 6. Purity: 82% (18% toluene); calc. yield 71%. ¹⁹F NMR (CDCl₃): −80.96 (3F, t, 8.5 Hz), −124.10 (1F, d quint., 282, 9.1, Hz), −125.20 (1F, q quint., 282, 9.0), −128.04 (2F, d, 4.2 Hz) ppm; ¹H NMR (CDCl₃): 3.79 (1H, t, 9.0 Hz) ppm.

### Example 8. Trans-2-(perfluorobutyl)-3-(perfluoroethyl)oxirane

[chemical structure]

A mixture of 20 mL of sodium hypochlorite, 6.0 mL of o-xylene, 0.35 g tetrabutylphosphonium bromide (10 mol %), and 4.0 g of (E)-1,1,1,2,2,5,5,6,6,7,7,8,8,8-tetradecafluorooct-3-ene was agitated at ambient temperature for 24 h (conversion of olefin: 100%). The reaction mixture was then diluted with water and the organic layer was separated, washed by water, and dried over MgSO₄ and analyzed by NMR. Isolated material (9 g) was found to be a mixture of desired epoxide, o-xylene (ratio 35:65) and some tetrabutylphosphonium bromide (NMR). ¹⁹F NMR (CDCl₃): −81.27 (3F, tt, 9.9, 2.7 Hz), −84.38 (3F,$), −122.99 (1F, dq, 279.0, 9.8 Hz), −124.80 (2F, m), −126.42 (2F, m), −125.20 (1F, q quint., 282, 9.0), −127.23 (1F, dd., 276.7, 10.5 Hz) −127.84 (1F, dd, 276.7, 9.0 Hz) ppm; ¹H NMR (CDCl₃): 3.73 (1H, t, 9.2 Hz), 3.79 (1H, t, 9.0 Hz) ppm.

### Example 9. Trans-2,3-bis(perfluorobutyl)oxirane

[chemical structure]

A mixture of 20 mL of sodium hypochlorite, 6.0 mL of o-xylene, 0.3 g tetrabutylphosphonium bromide (10 mol %), and 4.0 g of (E)-1,1,1,2,2,3,3,4,4,7,7,8,8,9,9,10,10,10-octadecafluorodec-5-ene was agitated at ambient temperature for 24 h (conversion of olefin was 90%). The reaction mixture was then diluted with water and the organic layer was separated, washed by water, and dried over MgSO₄ and analyzed by NMR. Isolated material (9.5 g) was found to be a mixture of desired epoxide, starting olefin, o-xylene (ratio 33.7:4.2:62.1) and some tetrabutylphosphonium bromide (NMR). ¹⁹F NMR (CDCl₃): −81.17 (3F, tt, 9.8, 2.7 Hz), −123.20 (1F, dq, 280.1, 12.7 Hz), −124.54 (1F, dq, 280.1, 10.2 Hz), −124.68 (2F, m), −124.68 (2F, m), −126.36 (2F, m) ppm; ¹H NMR (CDCl₃): 3.77 (t, 8.9 Hz) ppm

### Example 10. 2-(2,2,2-Trifluoroethoxy)-3-fluoro-2-(trifluoromethyl)-3-(perfluoroethyl)oxirane (Mixture of Z and E Isomers)

[chemical structure]

A mixture of 260 mL of sodium hypochlorite, 3.4 g tetrabutylammonium hydrogensulfate, and 60 g of 2-(2,2,2-trifluoroethoxy)-1,1,1,3,4,4,5,5,5-nonafluoropent-2-ene was agitated at ambient temperature for 24 h. The reaction mixture was then diluted with 500 mL of water and the organic layer was separated, washed by water, dried over MgSO₄, and distilled to afford 28 g of the desired epoxide (mixture of isomers), b.p. 91-94° C. (main 93° C., mixture of isomers, ratio-95:5). Yield: 46%. Major isomer: ¹⁹F NMR (CDCl₃): −72.89 (3F, d, 14.3 Hz), −75.08 (3F, m), −82.88 (3F, t, 8.5 Hz), −124.10 (1F, d quint., 282, 9.1, Hz), −124.83 (1F, dt., 286.1, 3.7), −151.79 (1F m), ppm; ¹H NMR (CDCl₃): 4.10 (1H, m), 4.24 (1H, m) ppm.

### Example 11. 2,3-Dichloro-2,3-bis(trifluoromethyl)oxirane (Mixture of Isomers)

[chemical structure]

A 1000 mL round bottom flask was equipped with a magnetic stir bar, a dry ice condenser, and a thermowell. The flask was charged with acetonitrile (50 mL, 0.95 moles), 15% w/w chilled sodium hypochlorite (450 mL, 6.71 moles), and the tetrabutylammonium hydrogen sulfate (5 mol %, 6.0 grams). The reaction mixture was stirred at room temperature. An addition funnel containing (E/Z)-2,3-dichloro-1,1,1,4,4,4-hexafluorobut-2-ene (0.33 moles, 78 grams) was fixed onto the reaction flask; olefin was added dropwise to the reaction mixture over a period of 30 minutes. A slight exotherm was observed once the internal temperature reached 25° C. The reaction mixture stirred for 48 h. The contents of the flask were transferred to a separatory funnel and the organic layer was separated, dried over magnesium sulfate and filtered into a clean round bottom flask. The filtrate was then subjected to reduced pressure, crude product was collected in a dry ice cold trap and subsequently distilled to provide the desired product, with b.p. 68-69, (15.73 g, yield 18.9%).

### Examples 12A-12B. Trans-2-fluoro-3-(perfluoropropan-2-yl)oxirane (Example 12A) and cis-2-fluoro-3-(perfluoropropan-2-yl)oxirane (Example 12B)

[chemical structure]

A 1000 mL round bottom flask was equipped with a magnetic stir bar, a dry ice condenser, and a thermowell. The flask was charged with xylenes or toluene (100 mL), 15% w/w chilled sodium hypochlorite (500 mL, 6.71 moles), and Aliquat® 336 (5 mol %, 5 grams). The reaction mixture was stirred at 0° C. for the entire reaction. An addition funnel containing olefin (trans or cis-isomers of 1,3,4,4,4-pentafluoro-3-(trifluoromethyl)but-1-ene, 0.24 moles, 52.6 grams) was fixed onto the reaction flask; olefin was added dropwise to the reaction mixture over a period of 30 minutes. A slight exotherm was observed once addition of the olefin was started. The reaction mixture was sampled every hour over the first 6 hours and then stirred overnight. After 24 h, the contents of the flask were transferred to a separatory funnel and the organic layer was separated, dried with magnesium sulfate and filtered into a clean round bottom flask. The crude product was transferred into cold trap (−78° C.) under reduced pressure and distilled at atmospheric pressure.

Example 12A (xylenes as a solvent): E-isomer, b.p. 55.5° C.; yield 47.8%. ¹⁹F NMR (CDCl₃): −75.60 (3F, quint, 8.8 Hz), −75.77 (3F, quint., 8.7 Hz), −156.83 (1F, dd, 83.2, 2.8 Hz), −195.66 (1F, d, sept., 20.3, 8.1 Hz) ppm; ¹H NMR (CDCl₃): 3.67 (1H, dd, 20.3, 3.6 Hz), 5.64 (d, 83.2 Hz) ppm.

Example 12B (xylenes as a solvent): E-isomer b.p. 88-89° C.; yield 17%, purity 80%, contained 20% of xylenes). ¹⁹F NMR (CDCl₃): −75.41 (3F, m), −76.26 (3F, m), −163.20 (1F, dddq, 83.9, 42.9, 5.5, 3.7 Hz), −195.41 (1F, d.d. quint., 42.9, 19.4, 3.7 Hz) ppm ¹H NMR (CDCl₃): 3.24 (1H, d, 19.4 Hz), 5.67 (1H, dm, 83.9, 1.0 Hz) ppm;

### Example 13. 2,2,3,3,4,4-Hexafluoro-6-oxa-bicyclo[3.1.0]hexane

[chemical structure]

The reaction was performed using 120 mL of NaOCl, 30 mL of ACN, 2.5 g of tetrabutylammonium hydrogen sulfate (˜3 mol %), and 20 g (0.11 mol) of 3,3,4,4,5,5-hexafluorocyclopent-1-ene. The reaction was mildly exothermic reaction at 20-27° C. The reaction mixture was agitated at 20-25° C. for 4 h, then the organic phase was separated, washed by water, and dried over MgSO₄. The crude mixture was distilled to give 16 g of epoxide as single Z-isomer (NMR), b.p. 98-101° C. (main 100-101° C.), 98% purity, containing ˜2% of starting olefin, GC, NMR). Calculated yield: 75%. ¹⁹F (CDCl₃): −114.55 (1F, dm, 248.5 Hz), −115.22 (2F, d, 262.2 Hz), −125.75 (2F, dm, 263.2 Hz), −140.61 (1F, dm, 248.5 Hz) ppm; ¹H NMR (CDCl₃): 3.99 (m) ppm; ¹³C {H}: 51.16 (m), 111.93 (m, two CF₂-groups); MS (m/z): 192 (M⁺, C₅H₂F₆O⁺).

### Example 14. 2,2,3,3-Tetrafluoro-5-oxabicyclo[2.1.0]pentane

[chemical structure]

The starting material for this reaction, 3,3,4,4-tetrafluorocyclobut-1-ene, was prepared from 1-chloro-2,2,3,3-tetrafluorocyclobutane according to previously reported procedures (see e.g., Coffman et al, J. Am. Chem. Soc. 1949, 71:490-496). The title product was prepared using 120 mL of NaOCl, 30 mL of xylenes, 2.5 g of tetrabutylammonium hydrogen sulfate (˜3 mol %), and 30 g (0.24 mol) of 3,3,4,4-tetrafluorocyclobut-1-ene, which was added dropwise to the reaction mixture at 15-20° C. Mildly exothermic reaction at 20-27° C. was observed. The reaction mixture was agitated at 20-2° C. for 4 h (conversion of starting material 100%), then the organic phase was separated, washed by water, and dried over MgSO₄. Distillation of crude mixture gave 21 g (63%) of cis-epoxide as a single isomer (NMR), b.p. 73-74° C. (98% purity, containing ˜2% of xylenes, GC). ¹⁹F (CDCl₃): −117.77 (2F, dm, 204.0 Hz), −127.49 (2F, dm, 204.0 Hz) ppm; ¹H NMR (CDCl₃): 4.38 (m) ppm; MS (m/z): 142 (M⁺, C₄H₂F₆O⁺);

### Example 15. General Procedure for Preparation Epoxides of Perfluoroolefins

A 500 mL round bottom flask was equipped with a magnetic stir bar, a dry ice condenser, and a thermowell. The flask was charged with acetonitrile (25 mL, 0.47 moles), 15% w/w chilled sodium hypochlorite (250 mL, 3.72 moles), and the desired phase transfer catalyst (e.g., tetrabutylammonium hydrogen sulfate, 5 mol %, 3.06 g). The reaction mixture was stirred at room temperature. An addition funnel containing olefin (0.18 mole, typically a mixture of 18-90% of trans- and 10-20% cis-isomers) was fixed onto the reaction flask; olefin was added dropwise to the reaction mixture over a period of 30 minutes. A slight exotherm was observed once the internal temperature reached 25° C. The reaction temperature was maintained at <25° C. using a cooling bath. The reaction mixture was sampled every hour over the first 6 hours. Once the reaction was complete, the contents of the flask were transferred to a separatory funnel and organic layer was separated, washed with water, and dried over magnesium sulfate. The desired epoxides were isolated as a mixture of trans- and cis-isomers (determined by NMR), with ratios very similar to those observed in the corresponding starting material.

### Example 16. 2,3-Difluoro-2-(perfluoroethyl)-3-(perfluoropropyl)oxirane (Mixture of Isomers)

[chemical structure]

The title compound was prepared according to the general procedure described in Example 15, using perfluoroheptene-3 (98% purity, 2% of perfluoroheptene-2) as starting material. The epoxide (mixture trans- and cis-isomers 98:2) was isolated in 75% yield, b.p. 80° C.

### Example 17. 2,3-Difluoro-2-(trifluoromethyl)-3-(perfluoropentyl)oxirane (Mixture of Isomers)

[chemical structure]

The title compound was prepared according to the general procedure described in Example 15, using perfluoroctene-2 as starting material. Purity of isolated epoxide was 95% (5% starting material); calc. yield: 84%; ratio trans-/cis-epoxides: 95:5.

### Example 18. Comparative Example—Effect of Phase Transfer Catalyst on Conversion Perfluoroheptene-3 in Synthesis of 2,3-difluoro-2-(perfluoroethyl)-3-(perfluoropropyl)oxirane

[chemical structure]

In 20 mL sample vial equipped with magnetic stir bar was added 15 mL of NaOCl solution and 3.5 g of perfluoroheptene-3 (98% purity) and 1 mL of acetonitrile. The sample vial was closed, placed on magnetic stir plate, the agitation speed was set to 1500 rpm and the reaction mixture was agitated at ambient temperature. After for 4 h the agitation was stopped and the reaction mixture (organic layer) was analyzed by GC/MS. The crude reaction mixture was shown to contain <5 wt % of the corresponding epoxide. At this time, 0.2 g of the phase-transfer catalyst, (C₄H₉)₄N⁺HSO₄⁻, was added to the reaction mixture and the agitation (1500 rpm) of the reaction mixture was continued at ambient temperature for additional 4 h. Subsequent GC/MS and ¹⁹F-NMR analysis showed that the reaction mixture (organic layer) contained starting material and the corresponding epoxide in a 5:95 ratio, demonstrating 95% conversion of the olefin.

### Example 19. Comparative Example—Effect of Solvent on Conversion of Perfluoroheptene-3 in Synthesis of 2,3-Difluoro-2-(perfluoroethyl)-3-(perfluoropropyl)oxirane

[chemical structure]

In 20 mL sample vial equipped with magnetic stir bar was added 15 mL of NaOCl solution, 3.5 g of perfluoroheptene-3 (98% purity), and 0.2 g of the phase-transfer catalyst, (C₄H₉)₄N⁺HSO₄⁻. The sample vial was closed, placed on magnetic stir plate, the agitation speed was set to 1500 rpm and the reaction mixture was agitated at ambient temperature. After 4 h the agitation was stopped and the reaction mixture (organic layer) was analyzed by GC/MS. The crude reaction mixture was shown to contain <5 wt % of the corresponding epoxide. At this time, 1 mL of acetonitrile was added to the reaction mixture and the agitation (1500 rpm) was continued at ambient temperature for additional 4 h. Subsequent GC/MS and ¹⁹F-NMR analysis showed that the reaction mixture (organic layer) contained starting material and the corresponding epoxide in a 4:96 ratio, demonstrating 96% conversion of the olefin into the epoxide.

### Example 20. Comparative Example—Effect of Solvent on Conversion of Olefin in Synthesis of E-2,3-bis(perfluorobutyl)oxirane

[chemical structure]

In 20 mL sample vial equipped with magnetic stir bar was placed 15 mL of NaOCl solution, 2.0 g of (E)-1,1,1,2,2,3,3,4,4,7,7,8,8,9,9,10,10,10-octadecafluorodec-5-ene, and 0.2 g of the phase-transfer catalyst, Aliquat® 336. The sample vial was closed, placed on magnetic stir plate, the agitation speed was set to 1500 rpm and the reaction mixture was agitated at ambient temperature. After for 20 h the agitation was stopped and the reaction mixture (organic layer) was analyzed by GC/MS. The crude reaction mixture was shown to contain ˜10 wt % of the corresponding epoxide. At this point 2 mL of acetonitrile was added to the same reaction mixture and the agitation (1500 rpm) of the reaction mixture was continued at ambient temperature for additional 20 h. Subsequent GC/MS and ¹⁹F-NMR analysis showed that the reaction mixture (organic layer) contained starting material and the corresponding epoxide in a 6:94 ratio, demonstrating 94% conversion of the olefin into the epoxide.

### Example 21A. (E)-2,3-difluoro-2-(trifluoromethyl)oxirane

[chemical structure]

The title compound is prepared by a method analogous to that of Example 1, starting from (E)-1,2,3,3,3-pentafluoropentene (E-HFO-1225ye).

### Example 21B. (Z)-2,3-difluoro-2-(trifluoromethyl)oxirane

[chemical structure]

The title compound is prepared by a method analogous to that of Example 1, starting from (Z)-1,2,3,3,3-pentafluoropentene (Z-HFO-1225ye).

### Example 22. Representative Refrigerant Cycle Calculations

Table 1 shows refrigerant cycle calculations using a representative example epoxide provided herein (Example 2; 1234zeE-Epo) compared to several commercial refrigerants. The cycle calculations were performed using the following parameters: Condenser temperature: 37.78° C.; Evaporator temperature: 4.44° C.; 0 subcool; 6K superheat; 85% compressor efficiency.

TABLE 1

| | Relative | COP |
|---|---|---|
| | Capacity to | Relative to |
| | R123 | R123 |
| R-123 | 100% | 100% |
| 1336mzz | 78% | 97% |
| (Z-isomer) | | |
| 1233zd | 139% | 99% |
| (E-isomer) | | |
| 245fa | 157% | 98% |
| 1336mzz | 199% | 96% |
| (E-isomer) | | |
| 1234zeE-Epo | 137% | 99% |

1234zeE-Epo provides cooling capacity equivalent to HCFO-1233zd-E and equivalent COP (measure of energy efficiency) also, which is better performance than the other refrigerants listed as compared to R-123 cooling performance.

### Example 23. Fire Extinguishment Utilizing trans-2,3-bis(trifluoromethyl)oxirane

The concentration of trans-2,3-bis(trifluoromethyl)oxirane required to extinguish flames of n-heptane was determined using the cup burner method as described in NFPA 2001, Standard on Clean Agent Fire Extinguishing Systems, 2018 Edition, Appendix B (NFPA). Trans-2,3-bis(trifluoromethyl)oxirane vapor was mixed with air and introduced to the flame, with the trans-2,3-bis(trifluoromethyl)oxirane concentration being slowly increased until the flow was just sufficient to cause extinction of the flame. The extinguishing concentration for n-heptane was determined to be 5.7% v/v trans-2,3-bis(trifluoromethyl)oxirane.

### Example 24. Inhalation Approximate Lethal Concentration (ALC) Study

An inhalation approximate lethal concentration (ALC) study was conducted in three groups of 2 male and 2 female (nulliparous and non-pregnant) Crl:CD(SD) rats each that were exposed whole-body for 1 hour to target concentrations of 0.2 and 1% v/v (2R,3S)-2,3-bis(trifluoromethyl)oxirane. The test substance was 99.5% pure as determined by GC/MS. The test atmospheres were generated by flash evaporation of the liquid test substance in air. The test substance was metered into a heated round-bottom, flash evaporation flask which was heated to 150-175° C. to vaporize the test substance. Houseline generation air was metered to the round-bottom flask and carried the vapor and air mixture into a glass transfer tube that led into the glass exposure chamber. Chamber concentrations of (2R,3S)-2,3-bis(trifluoromethyl)oxirane were controlled by varying the test substance feed rate or chamber airflow to the round-bottom flask and vapor atmospheric concentrations of (2R,3S)-2,3-bis(trifluoromethyl)oxirane were determined by gas chromatography/flame ionization detector (GC/FID) analysis. Animals were observed for mortality and response to alerting stimuli (startle response) at least 3 times during the exposures. Rats were observed daily for mortality and were weighed and observed for clinical signs of toxicity during the 14-day recovery periods.

Animals in the 0.2 and 1% (target concentrations) groups were exposed for 1 hour to vapor concentrations of 0.2050±0.0103 (mean±standard deviation) and 1.0000±0.0581% (2R,3S)-2,3-bis(trifluoromethyl)oxirane, respectively. Fractional mortalities (number of deaths/number exposed) for the 0.2050 and 1.0000% exposures were 0/4 and 0/4, respectively. Rats exposed to 0.2050% displayed no clinical signs of toxicity throughout the entire study and displayed normal startle responses during the exposure. All rats in the 1.0000% exposure group displayed normal startle response for the first 20 minutes of the exposure followed by decreased startle response at approximately 30 minutes of exposure and after 50 minutes of exposure, the rats were prostrate and unresponsive to sound stimulus. As their startle response decreased, their activity level and breathing rate also decreased. Fifteen minutes after the test substance flow was terminated, all rats displayed normal startle response and displayed no abnormal clinical signs. One female rat in the 0.2050% exposure group and another female rat in the 1.0000% exposure group each displayed 3-gram bodyweight losses on the day after the exposure. There were no other bodyweight losses observed in any rats in these exposure groups throughout their recovery periods.

### Example 25. Use of E-1336mzz Epoxide as a Working Fluid for High Temperature Heat Pumps

Table 2 compares the performance of a high temperature heat pump cycle, lifting heat from a temperature of 95° C. to 145° C., using E-1336mzz Epoxide (trans-2,3-bis(trifluoromethyl)oxirane) as the working fluid, to the performance of the same cycle with HFC-245fa as the working fluid. As shown in Table 2, use of E-1336mzz Epoxide increased the cycle energy efficiency, as measured in terms of the Coefficient of Performance for heating, COPh, by 5.4%, while at the same time reducing the working fluid GWP by 28%. Moreover, the condenser pressure with E-1336mzz Epoxide could easily be confined with widely available low-cost equipment while the higher condenser pressure with HFC-245fa would require more expensive equipment.

TABLE 2

| | | | E-1336mzz | |
|---|---|---|---|---|
| | Units | HFC-245fa | Epoxide | Change |
| NBP | oC | 15.3 | 35.0 | |
| Tcr | oC | 154.1 | 173.1 | |
| GWP | | 858 | 614 | −28% |
| Evaporator | oC | 95.0 | 95.0 | |
| Temperature | | | | |
| Condenser | oC | 145.0 | 145.0 | |
| Temperature | | | | |
| Suction Superheat | K | 20.0 | 20.0 | |
| Condenser Liquid | K | 20.0 | 20.0 | |
| Sub-cooling | | | | |
| Compressor | | 0.75 | 0.75 | |
| Efficiency | | | | |
| Condenser Pressure | Pa | 3,090,275 | 1,516,393 | |
| Evaporator Pressure | Pa | 1,129,379 | 561,366 | |
| COPₕ | | 5.589 | 5.890 | +5.4% |

### OTHER EMBODIMENTS

1. In some embodiments, the present application provides a composition for use in refrigeration, in air conditioning, in heating, in heat transfer, in conversion of heat into mechanical work in a power cycle, as a foam blowing agent, as a solvent, or in preventing or quenching an electric discharge, comprising a refrigerant component, an air conditioning component, a heating component, a heat transfer component, a working fluid component, a blowing agent component, a solvent component, or a dielectric component, respectively, which is a compound of Formula (I):

[chemical structure]

wherein:
R¹ and R⁴ are each independently H, Cl, F, Br, I, a partially fluorinated C₁₋₄ alkoxy, or a perfluorinated C₁₋₄ alkoxy; and
R² is selected from H, Cl, F, Br, I, a partially fluorinated C₁₋₁₀ alkyl, a perfluorinated C₁₋₁₀ alkyl, a partially fluorinated C₁₋₄ alkoxy, and a perfluorinated C₁₋₄ alkoxy;
wherein at least one of R¹, R², and R⁴ is not H;
R³ is selected from partially fluorinated C₁₋₁₀ alkyl and perfluorinated C₁₋₁₀ alkyl;
or alternatively, R² and R³ are each independently selected from partially fluorinated or perfluorinated C₁₋₅ alkylene, which together form a monocyclic ring.
2. The composition of embodiment 1, wherein:
   R¹ and R⁴ are each independently H, Cl, F, a partially fluorinated C₁₋₄ alkoxy, or a perfluorinated C₁₋₄ alkoxy;
   R² is H, F, partially fluorinated C₁₋₁₀ alkyl, or perfluorinated C₁₋₁₀ alkyl; wherein at least one of R¹, R², and R⁴ is not H; and R³ is selected from partially fluorinated C₁₋₁₀ alkyl and perfluorinated C₁₋₁₀ alkyl.
3. The composition of embodiment 1, wherein R¹ and R⁴ are each independently H, Cl, or F; R² is F, CF₃, CF₂CF₃, CF(CF₃)₂, CF₂CF₂CF₃, CF₂CF₂CF₂CF₃, or CF₂CF₂CF₂CF₂CF₃; and R³ is CF₃, CF₂CF₃, CF(CF₃)₂, CF₂CF₂CF₃, CF₂CF₂CF₂CF₃, or CF₂CF₂CF₂CF₂CF₃.
4. The composition of embodiment 1, wherein R¹ and R⁴ are each independently H, Cl, F, a partially fluorinated C₁₋₄ alkoxy, or a perfluorinated C₁₋₄ alkoxy; and R² and R³ are each independently selected from partially fluorinated or perfluorinated C₁₋₅ alkylene, which together form a monocyclic ring.
5. The composition of embodiment 1, wherein R¹ and R⁴ are H; R² is partially fluorinated or perfluorinated C₁₋₂ alkylene; and R³ is partially fluorinated or perfluorinated C₁₋₂ alkylene; wherein said R² and R³ are taken together form a 4-6 membered monocyclic ring.
6. The composition of embodiment 1, wherein R¹ and R⁴ are H; R² and R³ are independently selected from —CF₂— and —CF₂CF₂—, wherein said R² and R³ are taken together form a 4-6 membered monocyclic ring.
7. The composition of embodiment 1, wherein the compound of Formula (I) is selected from:
   2,3-difluoro-2-(trifluoromethyl)oxirane;
   2-fluoro-3-(trifluoromethyl)oxirane;
   2,3-bis(trifluoromethyl)oxirane;
   2-(trifluoromethyl)-3-(perfluoroethyl)oxirane;
   2-(trifluoromethyl)-3-(perfluoropropan-2-yl)oxirane;
   2,3-bis(perfluoropropyl)oxirane;
   2-(perfluorobutyl)-3-(perfluoroethyl)oxirane;
   2,3-bis(perfluorobutyl)oxirane;
   2-(2,2,2-Trifluoroethoxy)-3-fluoro-2-(trifluoromethyl)-3-(perfluoroethyl)oxirane;
   2,3-dichloro-2,3-bis(trifluoromethyl)oxirane;
   2-fluoro-3-(perfluoropropan-2-yl)oxirane;
   2,2,3,3,4,4-hexafluoro-6-oxa-bicyclo[3.1.0]hexane;
   2,2,3,3-tetrafluoro-5-oxabicyclo[2.1.0]pentane;
   2,3-difluoro-2-(perfluoroethyl)-3-(perfluoropropyl)oxirane; and
   2,3-difluoro-2-(trifluoromethyl)-3-(perfluoropentyl)oxirane;
   or a mixture thereof
8. The composition of embodiment 1, wherein the compound of Formula (I) is selected from the group consisting of:
   (Z)-2,3-difluoro-2-(trifluoromethyl)oxirane;
   (E)-2,3-difluoro-2-(trifluoromethyl)oxirane;
   cis-2-fluoro-3-(trifluoromethyl)oxirane;
   trans-2-fluoro-3-(trifluoromethyl)oxirane;
   trans-2,3-bis(trifluoromethyl)oxirane;
   cis-2,3-bis(trifluoromethyl)oxirane;
   trans-2-(trifluoromethyl)-3-(perfluoroethyl)oxirane;
   trans-2-(trifluoromethyl)-3-(perfluoropropan-2-yl)oxirane;
   trans-2,3-bis(perfluoropropyl)oxirane;
   trans-2-(perfluorobutyl)-3-(perfluoroethyl)oxirane;
   trans-2,3-bis(perfluorobutyl)oxirane;
   (Z)-2-(2,2,2-Trifluoroethoxy)-3-fluoro-2-(trifluoromethyl)-3-(perfluoroethyl)oxirane;
   (E)-2-(2,2,2-Trifluoroethoxy)-3-fluoro-2-(trifluoromethyl)-3-(perfluoroethyl)oxirane;
   cis-2,3-dichloro-2,3-bis(trifluoromethyl)oxirane;
   trans-2,3-dichloro-2,3-bis(trifluoromethyl)oxirane;
   trans-2-fluoro-3-(perfluoropropan-2-yl)oxirane;
   cis-2-fluoro-3-(perfluoropropan-2-yl)oxirane;
   cis-2,2,3,3,4,4-hexafluoro-6-oxa-bicyclo[3.1.0]hexane;
   cis-2,2,3,3-tetrafluoro-5-oxabicyclo[2.1.0]pentane;
   cis-2,3-difluoro-2-(perfluoroethyl)-3-(perfluoropropyl)oxirane;
   trans-2,3-difluoro-2-(perfluoroethyl)-3-(perfluoropropyl)oxirane;
   cis-2,3-difluoro-2-(trifluoromethyl)-3-(perfluoropentyl)oxirane; and
   trans-2,3-difluoro-2-(trifluoromethyl)-3-(perfluoropentyl)oxirane;
   or a mixture thereof
9. The composition of any one of embodiments 1-8, wherein the composition is a composition for use in refrigeration or air conditioning, wherein the composition comprises said refrigerant component or said air conditioning component, respectively.
10. The composition of embodiment 9, wherein the refrigerant component or air conditioning component is a compound of Formula (I) selected from (Z)-2,3-difluoro-2-(trifluoromethyl)oxirane, (E)-2,3-difluoro-2-(trifluoromethyl)oxirane, trans-2-fluoro-3-(trifluoromethyl)oxirane, and trans-2,3-bis(trifluoromethyl)oxirane, or a mixture thereof 11. The composition of any one of embodiments 9-10 wherein the composition further comprises difluoromethane (HFC-32), 1,1-difluoroethane (HFC-152a), 1,1,1,2-tetrafluoroethane (HFC-134a), 1,1,2,2-tetrafluoroethane (HFC-134), pentafluoroethane (HFC-125), 1,1,1-trifluoroethane (HFC-143a), 1,1,1,2,3,3,3-heptafluoropropane, (E)-1,3,3,3-tetrafluoroprop-1-ene (E-HFO-1234ze), 2,3,3,3-tetrafluoroprop-1-ene, (HFO-1234yf), (E)-1,1,1,4,4,4-hexafluorobut-2-ene (E-HFO-1336mzz), (Z)-1,1,1,4,4,4-hexafluorobut-2-ene (Z-HFO-1336mzz), (E)-1-chloro-3,3,3-trifluoropropene (E-1233zd), (Z)-1-chloro-3,3,3-trifluoropropene (Z-1233zd), (Z)-1-chloro-2,3,3,3-tetrafluoropropene (Z-HCFO-1224yd), (E)-1-chloro-2,3,3,3-tetrafluoropropene (E-HCFO-1224yd), 3,3,3-trifluoropropene (HFO-1243zf), (Z)-1,3,3,3-tetrafluoroprop-1-ene (Z-HFO-1234ze), (E)-1,2,3,3tetrafluoropropene (E-HFO-1234ye), (Z)-1,2,3,3tetrafluoropropene (Z-HFO-1234ye), (E)-1,1,1,4,4,5,5,5-octafluoro-2-pentene (E-HFO-1438mzz), (Z)-1,1,1,4,4,5,5,5-octafluoro-2-pentene (Z-HFO-1438mzz), (E)-1,3,4,4,4-pentafluoro-3-(trifluoromethyl)but-1-ene (E-HFO-1438ezy), (Z)-1,3,4,4,4-pentafluoro-3-(trifluoromethyl)but-1-ene (Z-HFO-1438ezy), 1,1,1,3,3-pentafluoropropane (HFC-245fa), 1-methoxyheptafluoropropane (HFE-7000), 1,1,1,3,3-pentafluorobutane (HFC-365mfc), 1,1,1,2,2,3,3,4,4-nonafluoro-4-methoxybutane (HFE-7100), trans-1,2-dichloroethylene, 2-bromo-1,1,1-trifluoro-2-propene, E-1,2-dichloro-1,2-difluoroethylene, Z-1,2-dichloro-1,2-difluoroethylene, perfluoroethyl perfluoroisopropyl ketone (F-ethyl isopropyl ketone), E-HFO-1,2,3,3,3-pentafluoropropene (E-HFO-1225ye), Z-HFO-1,2,3,3,3-pentafluoropropene (Z-HFO-1225ye), CF₃I, carbon dioxide, nitrogen, and argon.
12. The composition of any one of embodiments 9-11, wherein the composition comprises about 0.1% to 100%, about 0.1% to about 99%, about 1% to about 99%, about 10% to 99%, about 10% to about 99%, about 20% to about 99%, about 30% to about 90%, about 40% to about 99%, about 50% to about 99%, about 60% to about 99%, about 70% to about 99%, about 80% to about 99%, about 90% to about 99%, about 40% to about 90%, about 50% to about 90%, about 60% to about 90%, about 70% to about 90%, about 60% to about 80%, or about 50% to about 70% w/w of the compound of Formula (I) or a mixture of compounds of Formula (I).
13. A process for producing cooling, comprising evaporating the refrigerant component in the composition of any one of embodiments 9-12 in the vicinity of a body to be cooled, and thereafter condensing said refrigerant component.
14. A process for replacing an incumbent refrigerant, comprising substantially replacing said incumbent refrigerant with a composition of any one of embodiments 9-12.
15. The composition of any one of embodiments 1-8, wherein the composition is a composition for use in heating, wherein the composition comprises the heating component.
16. The composition of embodiment 15, wherein the heat component is a compound of Formula (I) selected from (Z)-2,3-difluoro-2-(trifluoromethyl)oxirane, (E)-2,3-difluoro-2-(trifluoromethyl)oxirane, trans-2-fluoro-3-(trifluoromethyl)oxirane, trans-2,3-bis(trifluoromethyl)oxirane, trans-2-(trifluoromethyl)-3-(perfluoroethyl)oxirane, cis-2-fluoro-3-(trifluoromethyl)oxirane, trans-2-fluoro-3-(perfluoropropan-2-yl)oxirane, or a mixture thereof.
17. The composition of embodiment 15 or 16, further comprising difluoromethane (HFC-32), 1,1-difluoroethane (HFC-152a), 1,1,1,2-tetrafuoroethane (HFC-134a), 1,1,2,2-tetrafluoroethane (HFC-134), pentafluoroethane (HFC-125), 1,1,1-trifluoroethane (HFC-143a), 1,1,1,2,3,3,3-heptafluoropropane, (E)-1,3,3,3-tetrafluoroprop-1-ene (E-HFO-1234ze), 2,3,3,3-tetrafluoroprop-1-ene (HFO-1234yf), 3,3,3-trifluoroprop-1-ene (HFO-1234zf), (E)-1,1,1,4,4,4-hexafluorobut-2-ene (E-1336mzz), (Z)-1,1,1,4,4,4-hexafluorobut-2-ene (Z-1336mzz), (E)-1-chloro-3,3,3-trifluoropropene (E-1233zd), (Z)-1-chloro-3,3,3-trifluoropropene (Z-1233zd), (Z)-1-chloro-2,3,3,3-tetrafluoropropene (Z-HCFO-1224yd), (E)-1-chloro-2,3,3,3-tetrafluoropropene (E-HCFO-1224yd), (Z)-1,3,3,3-tetrafluoroprop-1-ene (Z-HFO-1234ze), (E)-1,2,3,3-tetrafluoropropene (E-HFO-1234ye), (Z)-1,2,3,3-tetrafluoropropene (Z-HFO-1234ye), (E)-1,1,1,4,4,5,5,5-octafluoro-2-pentene (E-HFO-1438mzz), (Z)-1,1,1,4,4,5,5,5-octafluoro-2-pentene (Z-HFO-1438mzz), (E)-1,3,4,4,4-pentafluoro-3-(trifluoromethyl)but-1-ene (E-HFO-1438ezy), (E)-1,3,4,4,4-pentafluoro-3-(trifluoromethyl)but-1-ene (Z-HFO-1438ezy), 1,1,1,3,3-pentafluoropropane (HFC-245fa), 1-methoxyheptafluoropropane (HFE-7000),1,1,1,3,3-pentafluorobutane (HFC-365mfc), 1,1,1,2,2,3,4,5,5,5-decafluoropentane (HFC-4310mee), 1,1,1,2,2,3,3,4,4-nonafluoro-4-methoxybutane (HFE-7100).
18. The composition of any one of embodiments 15-17, wherein the composition comprises about 0.1% to 100%, about 0.1% to about 99%, about 1% to about 99%, about 10% to 99%, about 10% to about 99%, about 20% to about 99%, about 30% to about 90%, about 40% to about 99%, about 50% to about 99%, about 60% to about 99%, about 70% to about 99%, about 80% to about 99%, about 90% to about 99%, about 40% to about 90%, about 50% to about 90%, about 60% to about 90%, about 70% to about 90%, about 60% to about 80%, or about 50% to about 70% w/w of the compound of Formula (I) or a mixture of compounds of Formula (I).
19. A process for producing heating, comprising condensing the composition of any one of embodiments 15-18 in the vicinity of a body to be heated, and thereafter evaporating said heating component.
20. The composition of any one of embodiments 1-8, wherein the composition is for use in heat transfer, wherein the working fluid component is a heat transfer component.
21. The composition of embodiment 20, wherein the heat transfer component is a compound of Formula (I) selected from (Z)-2,3-difluoro-2-(trifluoromethyl)oxirane, (E)-2,3-difluoro-2-(trifluoromethyl)oxirane, cis-2-fluoro-3-(trifluoromethyl)oxirane, trans-2-fluoro-3-(trifluoromethyl)oxirane, trans-2,3-bis(trifluoromethyl)oxirane, cis-2,3-bis(trifluoromethyl)oxirane, trans-2-(trifluoromethyl)-3-(perfluoroethyl)oxirane, trans-2-(trifluoromethyl)-3-(perfluoropropan-2-yl)oxirane, trans-2,3-bis(perfluoropropyl)oxirane, trans-2-(perfluorobutyl)-3-(perfluoroethyl)oxirane, trans-2,3-bis(perfluorobutyl)oxirane, (Z)-2-(2,2,2-Trifluoroethoxy)-3-fluoro-2-(trifluoromethyl)-3-(perfluoroethyl)oxirane, (E)-2-(2,2,2-Trifluoroethoxy)-3-fluoro-2-(trifluoromethyl)-3-(perfluoroethyl)oxirane, cis-2,3-dichloro-2,3-bis(trifluoromethyl)oxirane, trans-2,3-dichloro-2,3-bis(trifluoromethyl)oxirane, trans-2-fluoro-3-(perfluoropropan-2-yl)oxirane, cis-2-fluoro-3-(perfluoropropan-2-yl)oxirane, cis-2,2,3,3,4,4-hexafluoro-6-oxa-bicyclo[3.1.0]hexane, cis-2,2,3,3-tetrafluoro-5-oxabicyclo[2.1.0]pentane, cis-2,3-difluoro-2-(perfluoroethyl)-3-(perfluoropropyl)oxirane, trans-2,3-difluoro-2-(perfluoroethyl)-3-(perfluoropropyl)oxirane, cis-2,3-difluoro-2-(trifluoromethyl)-3-(perfluoropentyl)oxirane, and trans-2,3-difluoro-2-(trifluoromethyl)-3-(perfluoropentyl)oxirane, or a mixture thereof.
22. The composition of any one of embodiments 20-21, wherein the composition further comprises difluoromethane, 1,1-difluoroethane, 1,1,1,2-tetrafluoroethane, pentafluoroethane, 1,1,1,2,3,3,3-heptafluoropropane, (E)-1,3,3,3-tetrafluoroprop-1-ene (E-HFO-1234ze), 2,3,3,3-tetrafluoroprop-1-ene (HFO-1234yf), (E)-1,1,1,4,4,4-hexafluorobut-2-ene (E-HFO-1336mzz), (Z)-1,1,1,4,4,4-hexafluorobut-2-ene (Z-1336mzz), (E)-1-chloro-3,3,3-trifluoropropene (E-1233zd), (Z)-1-chloro-3,3,3-trifluoropropene (Z-1233zd), (Z)-1-chloro-2,3,3,3-tetrafluoropropene (Z-HCFO-1224yd), (E)-1-chloro-2,3,3,3-tetrafluoropropene (E-HCFO-1224yd), (Z)-1,3,3,3-tetrafluoroprop-1-ene (Z-HFO-1234ze), (E)-1,2,3,3-tetrafluoropropene (E-HFO-1234ye), (Z)-1,2,3,3-tetrafluoropropene (Z-HFO-1234ye), (E)-1,1,1,4,4,5,5,5-octafluoro-2-pentene (E-HFO-1438mzz), (Z)-1,1,1,4,4,5,5,5-octafluoro-2-pentene (Z-HFO-1438mzz), (E)-1,3,4,4,4-pentafluoro-3-(trifluoromethyl)but-1-ene (E-HFO-1438ezy), (Z)-1,3,4,4,4-pentafluoro-3-(trifluoromethyl)but-1-ene (Z-HFO-1438ezy), 1,1,1,3,3-pentafluoropropane (HFC-245fa), 1-methoxyheptafluoropropane (HFE-7000),1,1,1,3,3-pentafluorobutane (HFC-365mfc), 1,1,1,2,2,3,4,5,5,5-decafluoropentane (HFC-4310mee), 1,1,1,2,2,3,3,4,4-nonafluoro-4-methoxybutane (HFE-7100), methyl perfluoroheptene ether isomers, or (2E)-1,1,1,4,5,5,5-heptafluoro-4-(trifluoromethyl)pent-2-ene (HFO-153-10mzzy), or a mixture thereof.
23. The composition of any one of embodiments 20-22, wherein the composition comprises about 0.1% to 100%, about 0.1% to about 99%, about 1% to about 99%, about 10% to 99%, about 10% to about 99%, about 20% to about 99%, about 30% to about 90%, about 40% to about 99%, about 50% to about 99%, about 60% to about 99%, about 70% to about 99%, about 80% to about 99%, about 90% to about 99%, about 40% to about 90%, about 50% to about 90%, about 60% to about 90%, about 70% to about 90%, about 60% to about 80%, or about 50% to about 70% w/w of the compound of Formula (I) or a mixture of compounds of Formula (I).
24. A process for transferring heat from heat source to heat sink, comprising transporting the composition of any one of embodiments 20-23 from the heat source to the heat sink.
25. The composition of any one of embodiments 1-8, wherein the composition is a composition for conversion of heat into mechanical work in a power cycle, wherein the composition comprises said working fluid component.
26. The composition of embodiment 25, wherein the working fluid component is a compound of Formula (I) selected from (Z)-2,3-difluoro-2-(trifluoromethyl)oxirane, (E)-2,3-difluoro-2-(trifluoromethyl)oxirane, cis-2-fluoro-3-(trifluoromethyl)oxirane, trans-2-fluoro-3-(trifluoromethyl)oxirane, trans-2,3-bis(trifluoromethyl)oxirane, cis-2,3-bis(trifluoromethyl)oxirane, trans-2-(trifluoromethyl)-3-(perfluoroethyl)oxirane, trans-2-(trifluoromethyl)-3-(perfluoropropan-2-yl)oxirane, trans-2,3-bis(perfluoropropyl)oxirane, trans-2-(perfluorobutyl)-3-(perfluoroethyl)oxirane, trans-2,3-bis(perfluorobutyl)oxirane, (Z)-2-(2,2,2-Trifluoroethoxy)-3-fluoro-2-(trifluoromethyl)-3-(perfluoroethyl)oxirane, (E)-2-(2,2,2-Trifluoroethoxy)-3-fluoro-2-(trifluoromethyl)-3-(perfluoroethyl)oxirane, cis-2,3-dichloro-2,3-bis(trifluoromethyl)oxirane, trans-2,3-dichloro-2,3-bis(trifluoromethyl)oxirane, trans-2-fluoro-3-(perfluoropropan-2-yl)oxirane, cis-2-fluoro-3-(perfluoropropan-2-yl)oxirane, cis-2,2,3,3,4,4-hexafluoro-6-oxa-bicyclo[3.1.0]hexane, cis-2,2,3,3-tetrafluoro-5-oxabicyclo[2.1.0]pentane, cis-2,3-difluoro-2-(perfluoroethyl)-3-(perfluoropropyl)oxirane, trans-2,3-difluoro-2-(perfluoroethyl)-3-(perfluoropropyl)oxirane, cis-2,3-difluoro-2-(trifluoromethyl)-3-(perfluoropentyl)oxirane, and trans-2,3-difluoro-2-(trifluoromethyl)-3-(perfluoropentyl)oxirane, or a mixture thereof
27. The composition of any one of embodiments 25-26, wherein the composition further comprises difluoromethane, 1,1-difluoroethane, 1,1,1,2-tetrafluoroethane, pentafluoroethane, 1,1,1,2,3,3,3-heptafluoropropane, (E)-1,3,3,3-tetrafluoroprop-1-ene (E-HFO-1234ze), 2,3,3,3-tetrafluoroprop-1-ene (HFO-1234yf), (E)-1,1,1,4,4,4-hexafluorobut-2-ene (E-HFO-1336mzz), (Z)-1,1,1,4,4,4-hexafluorobut-2-ene (Z-1336mzz), (E)-1-chloro-3,3,3-trifluoropropene (E-1233zd), (Z)-1-chloro-3,3,3-trifluoropropene (Z-1233zd), (Z)-1-chloro-2,3,3,3-tetrafluoropropene (Z-HCFO-1224yd), (E)-1-chloro-2,3,3,3-tetrafluoropropene (E-HCFO-1224yd), (Z)-1,3,3,3-tetrafluoroprop-1-ene (Z-HFO-1234ze), (E)-1,2,3,3-tetrafluoropropene (E-HFO-1234ye), (Z)-1,2,3,3-tetrafluoropropene (Z-HFO-1234ye), (E)-1,1,1,4,4,5,5,5-octafluoro-2-pentene (E-HFO-1438mzz), (Z)-1,1,1,4,4,5,5,5-octafluoro-2-pentene (Z-HFO-1438mzz), (E)-1,3,4,4,4-pentafluoro-3-(trifluoromethyl)but-1-ene (E-HFO-1438ezy), (Z)-1,3,4,4,4-pentafluoro-3-(trifluoromethyl)but-1-ene (Z-HFO-1438ezy), 1,1,1,3,3-pentafluoropropane (HFC-245fa), 1-methoxyheptafluoropropane (HFE-7000),1,1,1,3,3-pentafluorobutane (HFC-365mfc), 1,1,1,2,2,3,4,5,5,5-decafluoropentane (HFC-4310mee), 1,1,1,2,2,3,3,4,4-nonafluoro-4-methoxybutane (HFE-7100).
28. The composition of any one of embodiments 25-26, wherein the composition comprises about 0.1% to 100%, about 0.1% to about 99%, about 1% to about 99%, about 10% to 99%, about 10% to about 99%, about 20% to about 99%, about 30% to about 90%, about 40% to about 99%, about 50% to about 99%, about 60% to about 99%, about 70% to about 99%, about 80% to about 99%, about 90% to about 99%, about 40% to about 90%, about 50% to about 90%, about 60% to about 90%, about 70% to about 90%, about 60% to about 80%, or about 50% to about 70% w/w of the compound of Formula (I) or a mixture of compounds of Formula (I).
29. A process for converting heat into mechanical work in a power cycle, comprising the steps of heating the composition of any one of embodiments 25-28 with a heat source to a temperature sufficient to pressurize the composition; and causing the pressurized composition to perform mechanical work.
30. The process of embodiment 29, wherein said step of heating the composition with a heat source further comprises heating the working fluid component to a temperature sufficient to vaporize the composition and form a pressurized vapor of the composition.
31. The process of embodiment 30, further comprises expanding said pressurized vapor of the composition through an expansion device to perform the mechanical work.
32. The process of any one embodiments 29-31, wherein the mechanical work is transmitted to an electrical generator to produce electrical power.
33. The process of embodiment 29, wherein said heat is converted to said mechanical work by using a sub-critical cycle comprising (a) compressing the composition to a pressure below its critical pressure; and (b) heating the compressed liquid composition from step (a) using heat supplied by the heat source to form a vapor composition; (c) expanding the heated composition from step (b) to generate mechanical work and lowering the pressure of the composition; (d) cooling the expanded composition from step (c) to form a cooled liquid composition; and (e) cycling cooled liquid composition from steps (d) to (a) to repeat the cycle.
34. The process of embodiment 29, wherein said heat is converted to said mechanical work by using a trans-critical cycle comprising: (a) compressing the composition above said working fluid's critical pressure; (b) heating compressed composition from step (a) using heat supplied by the heat source; (c) expanding heated composition from step (b) to generate mechanical work and lowering the pressure of the composition below its critical pressure; (d) cooling the expanded composition from step (c) to form a cooled liquid composition; and (e) cycling the cooled liquid composition from steps (d) to (a) to repeat the cycle.
35. The process of embodiment 29 wherein said heat is converted to mechanical work by using a super-critical cycle comprising: (a) compressing the composition from a pressure above its critical pressure to a higher pressure; (b) heating the compressed composition from step (a) using heat supplied by the heat source; (c) expanding the heated composition from step (b) to generate mechanical work and lowering the pressure of the composition to a pressure above its critical pressure; (d) cooling the expanded composition from step (c) to form a cooled composition above its critical pressure; and (e) cycling the cooled composition from steps (d) to (a) to repeat the cycle.
36. A process of converting heat to mechanical work in a Rankine cycle comprising the steps of: (a) vaporizing the composition of any one of embodiments 25-28 with a low temperature heat source; (b) expanding the resulting vapor through an expansion device to generate mechanical work; (c) cooling the resulting expanded vapor to condense the vapor into a liquid; (d) pumping the composition to said heat source to repeat the process.
37. The composition of any one of embodiments 1-8, wherein the composition is a composition for use as a foam blowing agent, wherein the composition comprises a blowing agent component which is a compound of Formula (I).
38. The composition of embodiment 37, wherein the blowing agent component is a compound of Formula (I) selected from (Z)-2,3-difluoro-2-(trifluoromethyl)oxirane, (E)-2,3-difluoro-2-(trifluoromethyl)oxirane, trans-2-fluoro-3-(trifluoromethyl)oxirane, trans-2,3-bis(trifluoromethyl)oxirane, and trans-2-(trifluoromethyl)-3-(perfluoroethyl)oxirane, or a mixture thereof
39. The composition of embodiment 37 or 38, wherein said composition further comprises with one or more of methyl formate, dimethoxymethane, E-1,1,1,4,4,4-hexafluoro-2-butene, Z-1,1,1,4,4,4-hexafluoro-2-butene, E-1-chloro-3,3,3-trifluoropropene, Z-1-chloro-3,3,3-trifluoropropene, 1,1,1,2-tetrafluoroethane, 1,1-difluoroethane, dimethyl ether, ethanol, n-propane, n-butane, isobutane, n-pentane, isopentane, cyclopentane, CFO-1112, 1,2-dichloro-1,2-difluoroethylene, carbon dioxide, or water.
40. The composition of any one of embodiments 37-39, wherein the composition comprises about 0.1% to 100%, about 0.1% to about 99%, about 1% to about 99%, about 10% to 99%, about 10% to about 99%, about 20% to about 99%, about 30% to about 90%, about 40% to about 99%, about 50% to about 99%, about 60% to about 99%, about 70% to about 99%, about 80% to about 99%, about 90% to about 99%, about 40% to about 90%, about 50% to about 90%, about 60% to about 90%, about 70% to about 90%, about 60% to about 80%, or about 50% to about 70% w/w of the compound of Formula (I) or a mixture of compounds of Formula (I).
41. A foamable composition for use in formation of a foam, comprising the foam blowing agent composition of any one of embodiments 37-39 and one or more additional components capable of reacting and/or foaming under the proper conditions to form a foam or cellular structure.
42. A process for forming a foam, comprising reacting or extruding the foamable composition of embodiment 41 under conditions effective to form a foam.
43. The process of embodiment 42, wherein the one or more additional components comprises an isocyanate, at least one polyol, and at least one catalyst.
44. The process of embodiment 42, wherein the one or more additional components comprises a resin, wherein said resin is polystyrene, polypropylene or polyethylene.
45. The process of any one of embodiments 42-44, wherein the composition further comprises a nucleating agent, a fire retardant, or a combination thereof
46. A process of forming a polyisocyanate-based foam, comprising reacting the foamable composition of embodiment 41, wherein the one or more additional components comprises at least one organic polyisocyanate and at least one active hydrogen-containing compound.
47. The process of embodiment 46, wherein the at least one active hydrogen-containing compound is preblended with the blowing agent composition before reacting with at least one polyisocyanate.
48. The process of embodiment 46 or 47, wherein the at least one active hydrogen-containing compound/blowing agent blend contains at least 2 to about 50 wt. % blowing agent, based on the total weight of active hydrogen-containing compound and blowing agent.
49. The process of embodiment 46 or 47, wherein the at least one active hydrogen-containing compound/blowing agent blend contains at least 2 to about 25 wt. % blowing agent, based on the total weight of active hydrogen-containing compound and blowing agent.
50. A method of producing a polyurethane foam, comprising reacting the foamable composition of embodiment 41, wherein the one or more additional components comprises at least one active hydrogen containing compound, which is mixed with the blowing agent composition to form a B side mixture, and at least one organic polyisocyanate that forms an A side mixture.
51. The process of embodiment 50, wherein the at least one organic polyisocyanate, the at least one active hydrogen-containing compound, and the blowing agent composition are blended simultaneously.
52. The process of embodiment 50 or 51, wherein the reacting step is performed in the presence of at least one catalyst.
53. The process of any one of embodiments 50-52, wherein the B side further comprises at least one auxiliary component, said auxiliary component selected from the group consisting of a surfactant, a flame retardant, a preservative, a colorant, an antioxidant, a reinforcing agent, a filler, an antistatic agent, or a combination thereof
54. The process of embodiment 53, wherein the at least surfactant is present in a range of from about 0.2 to about 5 parts surfactant per 100 parts by weight polyol.
55. The process of embodiment 53 or 54, wherein the at least one surfactant is a liquid or solid organosilicone compound, a polyethylene glycol ether of a long chain alcohol, a tertiary amine or alkanolamine salt of a long chain alkyl acid sulfate ester, an alkyl sulfonic ester, or an alkyl arylsulfonic acid.
56. The process of embodiment 53, wherein the at least one catalyst is present in a range of from about 0.1 to about 5 parts catalyst per 100 parts by weight of polyol.
57. A process of forming a thermoplastic foam, comprising the steps of: (a) forming a melt comprising a foamable composition, wherein said foamable composition comprises polystyrene, polyethylene, or polypropylene; (b) blending the blowing agent composition of any one of embodiments 37-39 with the melt to form the composition of embodiment 41 at nonfoaming temperatures and pressures as a plasticized melt; (c) passing the plasticized mass at a controlled rate, temperature and pressure through a die and into an expansion zone to form an extrudate; (d) allowing the extrudate to foam in the expansion zone, maintaining the expanding extrudate under such temperatures and pressures for a time sufficient for the viscosity of the extrudate to increase such that the cell size and density of the foam remain substantially unchanged and substantially free of ruptured cells at 25° C. and atmospheric pressure.
58. The process of embodiment 57, wherein the foamable composition further comprises a nucleating agent, a fire retardant, or a combination thereof
59. The process of embodiment 57, wherein the plasticized mass comprises about 80 to about 99 wt. % polystyrene resin about 2 to about 20 wt. % blowing agent component, and about 0.1 to about 5 wt. % nucleating agent.
60. The process of embodiment 57, wherein the plasticized mass comprises about 80 to about 99 wt. % polypropylene resin about 2 to about 20 wt. % blowing agent component, and about 0.1 to about 5 wt. % nucleating agent.
61. The process of embodiment 57, wherein the plasticized mass comprises about 80 to about 99 wt. % polyethylene resin, about 2 to about 20 wt. % blowing agent component, and about 0.1 to about 5 wt. % nucleating agent.
62. The composition of any one of embodiments 1-8, wherein the composition is a composition for use as a solvent, wherein the composition comprises the solvent component.
63. The composition of embodiment 62, wherein the composition is a fluid for removal of particulates from metal surfaces, a carrier fluids, a dewatering agent, a degreasing solvent or a defluxing solvent.
64. The composition of any one of embodiments 62-63, wherein the composition further comprises trans-1,2-dichloroethylene, cyclopentane, cyclohexane, methyl acetate, acetone, methyl formate, ethyl formate, ethyl acetate, heptane, methanol, ethanol, isopropyl alcohol, dimethyl carbonate, propylene carbonate, tertiary butyl acetate, or methyl ethyl ketone.
65. The composition of any one of embodiments 62-64, wherein the composition comprises about 0.1% to 100%, about 0.1% to about 99%, about 1% to about 99%, about 10% to 99%, about 10% to about 99%, about 20% to about 99%, about 30% to about 90%, about 40% to about 99%, about 50% to about 99%, about 60% to about 99%, about 70% to about 99%, about 80% to about 99%, about 90% to about 99%, about 40% to about 90%, about 50% to about 90%, about 60% to about 90%, about 70% to about 90%, about 60% to about 80%, or about 50% to about 70% w/w of the compound of Formula (I) or a mixture of compounds of Formula (I).
66. A process for dissolving a solute, comprising contacting and mixing said solute with a sufficient quantity of the composition according to any one of embodiments 62-65.
67. A process of cleaning a surface, comprising contacting the composition of any one of embodiments 62-65 with said surface.
68. A process for removing at least a portion of water from the surface of a wetted substrate, comprising contacting the substrate with the composition of any one of embodiments 62-65 and then removing the substrate from contact with the composition.
69. The process of embodiment 68, wherein composition further comprises at least one surfactant suitable for dewatering or drying of substrates.
70. A process for depositing a coating on a surface, comprising contacting the composition of any one of embodiments 62-65 with said surface, wherein the composition further comprises a depositable material.
71. The process of embodiment 70, wherein the depositable material comprises a fluorolubricant or a photoresist.
72. The composition of any one of embodiments 1-8, wherein the composition is for use in preventing or rapidly quenching an electric discharge, wherein the composition comprises said dielectric component.
73. The composition of embodiment 72, wherein the dielectric component is a compound of Formula (I) selected from (Z)-2,3-difluoro-2-(trifluoromethyl)oxirane, (E)-2,3-difluoro-2-(trifluoromethyl)oxirane, trans-2-fluoro-3-(trifluoromethyl)oxirane, and trans-2,3-bis(trifluoromethyl)oxirane, or a mixture thereof
74. A method for preventing or rapidly quenching an electric discharge in a space in a high voltage device comprising injecting a gaseous dielectric into said space, wherein said gaseous dielectric comprises the composition of any one of embodiments 72-73.
75. A composition for fire suppression or fire extinguishment, comprising (a) a fluoroepoxide selected from (Z)-2,3-difluoro-2-(trifluoromethyl)oxirane, (E)-2,3-difluoro-2-(trifluoromethyl)oxirane, trans-2-fluoro-3-(trifluoromethyl)oxirane, trans-2,3-bis(trifluoromethyl)oxirane, trans-2-(trifluoromethyl)-3-(perfluoroethyl)oxirane, cis-2-fluoro-3-(trifluoromethyl)oxirane, trans-2-fluoro-3-(perfluoropropan-2-yl)oxirane, or a mixture thereof; and (b) one or more of 2-bromo-1,1,1-trifluoro-2-propene, E-1,2-dichloro-1,2-difluoroethylene, Z-1,2-dichloro-1,2-difluoroethylene, E-1-chloro-3,3,3-trifluoropropene, Z-1-chloro-3,3,3-trifluoropropene, E-1,1,1,4,4,4-hexafluoro-2-butene, Z-1,1,1,4,4,4-hexafluoro-2-butene, perfluoroethyl perfluoroisopropyl ketone (F-ethyl isopropyl ketone), E-1,3,4,4,4-pentafluoro-3-(trifluoromethyl)-1-butene), E-1,2,3,3,3-pentafluoropropene, Z-1,2,3,3,3-pentafluoropropene, E-1-chloro-2,3,3,3-tetrafluoropropene, Z-1-chloro-2,3,3,3-tetrafluoropropene, CF₃I, carbon dioxide, nitrogen, and argon.
76. The composition of embodiment 75, wherein the composition comprises about 0.1% to 100%, about 0.1% to about 99%, about 1% to about 99%, about 10% to 99%, about 1% to about 99%, about 10% to about 99%, about 20% to about 99%, about 30% to about 90%, about 40% to about 99%, about 50% to about 99%, about 60% to about 99%, about 70% to about 99%, about 80% to about 99%, about 90% to about 99%, about 40% to about 90%, about 50% to about 90%, about 60% to about 90%, about 70% to about 90%, about 60% to about 80%, or about 50% to about 70% w/w of the fluoroepoxide of (a).
77. A process for extinguishing or suppressing a flame comprising dispensing the composition of embodiment 75 or 76 at said flame.
78. A system for preventing or suppressing a flame comprising a vessel containing the composition according to embodiment 75 or 76 and a nozzle to dispense said composition toward an anticipated or actual location of said flame.
79. A method of reducing the flammability of a fluid comprising adding the composition of embodiment 75 or 76 to the fluid.
80. A method of extinguishing or suppressing a fire in a total-flood application comprising: (a) providing an agent comprising the flame suppression composition of embodiment 75 or 76; (b) disposing the agent in a pressurized discharge system; and (c) discharging the agent into an area to extinguish or suppress fires in that area.
81. A method of extinguishing or suppressing a fire in a streaming application comprising: (a) providing an agent comprising the flame suppression composition of embodiment 75 or 76; (b) disposing the agent in a pressurized discharge system; and (c) discharging the agent towards the location of a fire to extinguish or suppress the fire.
82. A method of inerting an area to prevent a fire or explosion comprising: (a) providing an agent comprising the flame suppression composition of embodiment 75 or 76; (b) disposing the agent in a pressurized discharge system; and (c) discharging the agent into the area to prevent a fire or explosion from occurring.
83. A method of preventing a fire in an enclosed area, comprising: (a) detecting a potential fire or ignition source, (b) discharging an agent comprising the flame suppression composition of embodiment 75 or 76 into the enclosed area thereby preventing the fire, wherein the resultant atmosphere within the enclosed area will not sustain or initiate combustion but remains habitable.
84. A sprayable composition comprising a propellant component and a co-propellant component comprising a compound of Formula (I):

[chemical structure]

wherein:
R¹ and R⁴ are each independently H, Cl, F, Br, I, a partially fluorinated C₁₋₄ alkoxy, or a perfluorinated C₁₋₄ alkoxy; and
R² is selected from H, Cl, F, Br, I, a partially fluorinated C₁₋₁₀ alkyl, a perfluorinated C₁₋₁₀ alkyl, a partially fluorinated C₁₋₄ alkoxy, and a perfluorinated C₁₋₄ alkoxy;
wherein at least one of R¹, R², and R⁴ is not H;
R³ is selected from partially fluorinated C₁₋₁₀ alkyl and perfluorinated C₁₋₁₀ alkyl;
or alternatively, R² and R³ are each independently selected from partially fluorinated or perfluorinated C₁₋₅ alkylene, which together form a monocyclic ring.
85. The composition of embodiment 84, wherein the co-propellant component is a compound of Formula (I) selected from (Z)-2,3-difluoro-2-(trifluoromethyl)oxirane, (E)-2,3-difluoro-2-(trifluoromethyl)oxirane, trans-2-fluoro-3-(trifluoromethyl)oxirane, trans-2,3-bis(trifluoromethyl)oxirane, cis-2,3-bis(trifluoromethyl)oxirane, trans-2-(trifluoromethyl)-3-(perfluoroethyl)oxirane, trans-2-(trifluoromethyl)-3-(perfluoropropan-2-yl)oxirane, trans-2,3-bis(perfluoropropyl)oxirane, trans-2-(perfluorobutyl)-3-(perfluoroethyl)oxirane, trans-2,3-bis(perfluorobutyl)oxirane, 2-(2,2,2-Trifluoroethoxy)-3-fluoro-2-(trifluoromethyl)-3-(perfluoroethyl)oxirane, 2,3-dichloro-2,3-bis(trifluoromethyl)oxirane, trans-2-fluoro-3-(perfluoropropan-2-yl)oxirane, cis-2-fluoro-3-(perfluoropropan-2-yl)oxirane, 2,2,3,3,4,4-hexafluoro-6-oxa-bicyclo[3.1.0]hexane, 2,3,3-tetrafluoro-5-oxabicyclo[2.1.0]pentane, 2,3-difluoro-2-(perfluoroethyl)-3-(perfluoropropyl)oxirane, 2,3-difluoro-2-(trifluoromethyl)-3-(perfluoropentyl)oxirane, and trans-2,3-bis(perfluorobutyl)oxirane, or a mixture thereof
86. The composition of embodiment 84 or 85, wherein the propellant component comprises carbon dioxide, difluoromethane (CF₂H₂, HFC-32), trifluoromethane (CF-₃H, HFC-23), difluoroethane (CHF₂CH₃, HFC-152a), trifluoroethane (CH₃CF₃, HFC-143a; or CHF₂CH₂F, HFC-143), tetrafluoroethane (CF₃CH₂F, HFC-134a; or CF₂HCF₂H, HFC-134), pentafluoroethane (CF₃CF₂H, HFC-125), 1,3,3,3-tetrafluoro-1-propene (HFO-1234ze), 2,3,3,3-tetrafluoro-1-propene (HFO-1234yf), 1,2,3,3,3-pentafluoropropene (HFO-1225ye), 1,1,3,3,3-pentafluoropropene (HFO-1225ze), hydrocarbons (e.g., propane, butanes, or pentanes) and dimethyl ether, or mixtures thereof
87. The composition of any one of embodiments 84-86, wherein the composition further comprises one or more additional co-propellant components is selected from dichlorodifluoromethane (CFC-12), trichlorofluoromethane (CFC-11), octafluorocyclobutane (C-318), 1,2-dichloro-1,1,2,2-tetrafluoroethane (CFC-114), 1,1,2-trichloro-1,2,2-trifluoroethane (CFC-113), dimethoxyethane (DME), ethers (e.g., dimethyl ether or diethyl ether), hydrofluorocarbon (e.g., difluoromethane (HFC-32), 1,1,1,2-tetrafluoroethane (HFC-134a), HFC-134a, 1,1,2,2-tetrafluoroethane (HFC-134), 1,1-difluoroethan (HFC-152a), 1,1,1,3,3,3-hexafluoropropane (HFC-236fa), or 1,1,1,2,3,3,3-heptafluoropropane (HFC-227ea)), hydrocarbon (e.g., propane, cyclopropane, n-butane, isobutane, cyclobutene, n-pentane, 2-methylbutane, cyclopentane, n-hexane, 2-methylpentane, or 2,3-dimethylbutane), or a hydrofluoroolefin (e.g., E-1,3,3,3-tetrafluoro-1-propene (E-HFO-1234ze), or 2,3,3,3-tetrafluoro-1-propene (HFO-1234yf), and a hydrofluoroolefin (e.g., HFO-1225ye isomers, HFO-1234yf isomers, HFO-1234ze isomers, HFO-1336mzz isomers, HCFO-1233zd isomers, or 1,2-dichloro-1,2-difluoroethylene isomers, 2,3,3,3-tetrafluoropropene (HFO-1234yf) (E)-1,3,3,3-tetrafluoroprop-1-ene (E-HFO-1234ze), (Z)-1,3,3,3-tetrafluoroprop-1-ene (Z-HFO-1234ze), or 2-chloro-3,3,3-trifluoropropene (HFO-1243zf), or inert gases (e.g., carbon dioxide, nitrogen or argon), or mixtures thereof
88. The composition of any one of embodiments 84-87, wherein the weight ratio of co-propellant component to propellant component is weight ratio of co-propellant component to propellant component is from about 5:95 to about 95:5.
89. A compound for use as an anesthetic agent, wherein the compound is a compound of Formula (I):

[chemical structure]

wherein:
R¹ and R⁴ are each independently H, Cl, F, Br, I, a partially fluorinated C₁₋₄ alkoxy, or a perfluorinated C₁₋₄ alkoxy; and
R² is selected from H, Cl, F, Br, I, a partially fluorinated C₁₋₁₀ alkyl, a perfluorinated C₁₋₁₀ alkyl, a partially fluorinated C₁₋₄ alkoxy, and a perfluorinated C₁₋₄ alkoxy;
wherein at least one of R¹, R², and R⁴ is not H;
R³ is selected from partially fluorinated C₁₋₁₀ alkyl and perfluorinated C₁₋₁₀ alkyl;
or alternatively, R² and R³ are each independently selected from partially fluorinated or perfluorinated C₁₋₅ alkylene, which together form a monocyclic ring.
90. A composition comprising a compound of Formula (I):

[chemical structure]

for use as an anesthetic agent, wherein:
R¹ and R⁴ are each independently H, Cl, F, Br, I, a partially fluorinated C₁₋₄ alkoxy, or a perfluorinated C₁₋₄ alkoxy; and
R² is selected from H, Cl, F, Br, I, a partially fluorinated C₁₋₁₀ alkyl, a perfluorinated C₁₋₁₀ alkyl, a partially fluorinated C₁₋₄ alkoxy, and a perfluorinated C₁₋₄ alkoxy;
wherein at least one of R¹, R², and R⁴ is not H;
R³ is selected from partially fluorinated C₁₋₁₀ alkyl and perfluorinated C₁₋₁₀ alkyl;
or alternatively, R² and R³ are each independently selected from partially fluorinated or perfluorinated C₁₋₅ alkylene, which together form a monocyclic ring.
91. A method of anesthetizing a subject, comprising administering to the subject an effective amount of a compound of Formula (I):

[chemical structure]

wherein:
R¹ and R⁴ are each independently H, Cl, F, Br, I, a partially fluorinated C₁₋₄ alkoxy, or a perfluorinated C₁₋₄ alkoxy; and
R² is selected from H, Cl, F, Br, I, a partially fluorinated C₁₋₁₀ alkyl, a perfluorinated C₁₋₁₀ alkyl, a partially fluorinated C₁₋₄ alkoxy, and a perfluorinated C₁₋₄ alkoxy;
wherein at least one of R¹, R², and R⁴ is not H;
R³ is selected from partially fluorinated C₁₋₁₀ alkyl and perfluorinated C₁₋₁₀ alkyl;
or alternatively, R² and R³ are each independently selected from partially fluorinated or perfluorinated C₁₋₅ alkylene, which together form a monocyclic ring.
92. The method of embodiment 91, wherein the method is performed in combination with a surgical procedure.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition for use in refrigeration, in air conditioning, in heating, in heat transfer, in conversion of heat into mechanical work in a power cycle, as a foam blowing agent, as a solvent, or in preventing or quenching an electric discharge, comprising a refrigerant component, an air conditioning component, a heating component, a heat transfer component, a working fluid component, a blowing agent component, a solvent component, or a dielectric component, respectively, which is a compound of Formula (I): [chemical structure]
wherein:
R¹ and R⁴ are each independently H, Cl, F, Br, I, a partially fluorinated C₁₋₄ alkoxy, or a perfluorinated C₁₋₄ alkoxy; and
R² is selected from H, Cl, F, Br, I, a partially fluorinated C₁₋₁₀ alkyl, a perfluorinated C₁₋₁₀ alkyl, a partially fluorinated C₁₋₄ alkoxy, and a perfluorinated C₁₋₄ alkoxy;
wherein at least one of R¹, R², and R⁴ is not H;
R³ is selected from partially fluorinated C₁₋₁₀ alkyl and perfluorinated C₁₋₁₀ alkyl;
or alternatively, R² and R³ are each independently selected from partially fluorinated or perfluorinated C₁₋₅ alkylene, which together form a monocyclic ring.

2. The composition of claim 1, wherein:
R¹ and R⁴ are each independently H, Cl, F, a partially fluorinated C₁₋₄ alkoxy, or a perfluorinated C₁₋₄ alkoxy;
R² is H, F, partially fluorinated C₁₋₁₀ alkyl, or perfluorinated C₁₋₁₀ alkyl;
wherein at least one of R¹, R², and R⁴ is not H; and
R³ is selected from partially fluorinated C₁₋₁₀ alkyl and perfluorinated C₁₋₁₀ alkyl.

3. The composition of claim 1, wherein R¹ and R⁴ are each independently H, Cl, or F; R² is F, CF₃, CF₂CF₃, CF(CF₃)₂, CF₂CF₂CF₃, CF₂CF₂CF₂CF₃, or CF₂CF₂CF₂CF₂CF₃; and R³ is CF₃, CF₂CF₃, CF(CF₃)₂, CF₂CF₂CF₃, CF₂CF₂CF₂CF₃, or CF₂CF₂CF₂CF₂CF₃.

4. The composition of claim 1, wherein R¹ and R⁴ are each independently H, Cl, F, a partially fluorinated C₁₋₄ alkoxy, or a perfluorinated C₁₋₄ alkoxy; and R² and R³ are each independently selected from partially fluorinated or perfluorinated C₁₋₅ alkylene, which together form a monocyclic ring.

5. The composition of claim 1, wherein R¹ and R⁴ are H; R² is partially fluorinated or perfluorinated C₁₋₂ alkylene; and R³ is partially fluorinated or perfluorinated C₁₋₂ alkylene; wherein said R² and R³ are taken together form a 4-6 membered monocyclic ring.

6. (canceled)

7. The composition of claim 1, wherein the compound of Formula (I) is selected from:
2,3-difluoro-2-(trifluoromethyl)oxirane;
2-fluoro-3-(trifluoromethyl)oxirane;
2,3-bis(trifluoromethyl)oxirane;
2-(trifluoromethyl)-3-(perfluoroethyl)oxirane;
2-(trifluoromethyl)-3-(perfluoropropan-2-yl)oxirane;
2,3-bis(perfluoropropyl)oxirane;
2-(perfluorobutyl)-3-(perfluoroethyl)oxirane;
2,3-bis(perfluorobutyl)oxirane;
2-(2,2,2-Trifluoroethoxy)-3-fluoro-2-(trifluoromethyl)-3-(perfluoroethyl)oxirane;
2,3-dichloro-2,3-bis(trifluoromethyl)oxirane;
2-fluoro-3-(perfluoropropan-2-yl)oxirane;
2,2,3,3,4,4-hexafluoro-6-oxa-bicyclo[3.1.0]hexane;
2,2,3,3-tetrafluoro-5-oxabicyclo[2.1.0]pentane;
2,3-difluoro-2-(perfluoroethyl)-3-(perfluoropropyl)oxirane; and
2,3-difluoro-2-(trifluoromethyl)-3-(perfluoropentyl)oxirane;
or a mixture thereof.

8. (canceled)

9. The composition of claim 1, wherein the composition is a composition for use in refrigeration or air conditioning, wherein the composition comprises said refrigerant component or said air conditioning component, respectively.

10-12. (canceled)

13. A process for producing cooling, comprising evaporating the refrigerant component in the composition of claim 9 in the vicinity of a body to be cooled, and thereafter condensing said refrigerant component.

14. A process for replacing an incumbent refrigerant, comprising substantially replacing said incumbent refrigerant with a composition of claim 9.

15-18. (canceled)

19. A process for producing heating, comprising condensing the composition of claim 9 in the vicinity of a body to be heated, and thereafter evaporating said heating component.

20. The composition of claim 1, wherein the composition is for use in heat transfer, wherein the working fluid component is a heat transfer component.

21. (canceled)

22. The composition of claim 20, wherein the composition further comprises difluoromethane, 1,1-difluoroethane, 1,1,1,2-tetrafluoroethane, pentafluoroethane, 1,1,1,2,3,3,3-heptafluoropropane, (E)-1,3,3,3-tetrafluoroprop-1-ene (E-HFO-1234ze), 2,3,3,3-tetrafluoroprop-1-ene (HFO-1234yf), (E)-1,1,1,4,4,4-hexafluorobut-2-ene (E-HFO-1336mzz), (Z)-1,1,1,4,4,4-hexafluorobut-2-ene (Z-1336mzz), (E)-1-chloro-3,3,3-trifluoropropene (E-1233zd), (Z)-1-chloro-3,3,3-trifluoropropene (Z-1233zd), (Z)-1-chloro-2,3,3,3-tetrafluoropropene (Z-HCFO-1224yd), (E)-1-chloro-2,3,3,3-tetrafluoropropene (E-HCFO-1224yd), (Z)-1,3,3,3-tetrafluoroprop-1-ene (Z-HFO-1234ze), (E)-1,2,3,3-tetrafluoropropene (E-HFO-1234ye), (Z)-1,2,3,3-tetrafluoropropene (Z-HFO-1234ye), (E)-1,1,1,4,4,5,5,5-octafluoro-2-pentene (E-HFO-1438mzz), (Z)-1,1,1,4,4,5,5,5-octafluoro-2-pentene (Z-HFO-1438mzz), (E)-1,3,4,4,4-pentafluoro-3-(trifluoromethyl)but-1-ene (E-HFO-1438ezy), (Z)-1,3,4,4,4-pentafluoro-3-(trifluoromethyl)but-1-ene (Z-HFO-1438ezy), 1,1,1,3,3-pentafluoropropane (HFC-245fa), 1-methoxyheptafluoropropane (HFE-7000),1,1,1,3,3-pentafluorobutane (HFC-365mfc), 1,1,1,2,2,3,4,5,5,5-decafluoropentane (HFC-4310mee), 1,1,1,2,2,3,3,4,4-nonafluoro-4-methoxybutane (HFE-7100), methyl perfluoroheptene ether isomers, or (2E)-1,1,1,4,5,5,5-heptafluoro-4-(trifluoromethyl)pent-2-ene (HFO-153-10mzzy), or a mixture thereof.

23. (canceled)

24. A process for transferring heat from heat source to heat sink, comprising transporting the composition of claim 20 from the heat source to the heat sink.

25-28. (canceled)

29. A process for converting heat into mechanical work in a power cycle, comprising the steps of heating the composition of claim 1 with a heat source to a temperature sufficient to pressurize the composition; and causing the pressurized composition to perform mechanical work.

30-35. (canceled)

36. A process of converting heat to mechanical work in a Rankine cycle comprising the steps of: (a) vaporizing the composition of claim 1 with a low temperature heat source; (b) expanding the resulting vapor through an expansion device to generate mechanical work; (c) cooling the resulting expanded vapor to condense the vapor into a liquid; (d) pumping the composition to said heat source to repeat the process.

37-40. (canceled)

41. A foamable composition for use in formation of a foam, comprising the foam blowing agent composition of claim 1 and one or more additional components capable of reacting and/or foaming under the proper conditions to form a foam or cellular structure.

42. A process for forming a foam, comprising reacting or extruding the foamable composition of claim 41 under conditions effective to form a foam.

43-65. (canceled)

66. A process for dissolving a solute, comprising contacting and mixing said solute with a sufficient quantity of the composition according to claim 1.

67. A process of cleaning a surface, comprising contacting the composition of claim 62 with said surface.

68. A process for removing at least a portion of water from the surface of a wetted substrate, comprising contacting the substrate with the composition of claim 1 and then removing the substrate from contact with the composition.

69-74. (canceled)

75. A composition for fire suppression or fire extinguishment, comprising (a) a fluoroepoxide selected from (Z)-2,3-difluoro-2-(trifluoromethyl)oxirane, (E)-2,3-difluoro-2-(trifluoromethyl)oxirane, trans-2-fluoro-3-(trifluoromethyl)oxirane, trans-2,3-bis(trifluoromethyl)oxirane, trans-2-(trifluoromethyl)-3-(perfluoroethyl)oxirane, cis-2-fluoro-3-(trifluoromethyl)oxirane, trans-2-fluoro-3-(perfluoropropan-2-yl)oxirane, or a mixture thereof; and (b) one or more of 2-bromo-1,1,1-trifluoro-2-propene, E-1,2-dichloro-1,2-difluoroethylene, Z-1,2-dichloro-1,2-difluoroethylene, E-1-chloro-3,3,3-trifluoropropene, Z-1-chloro-3,3,3-trifluoropropene, E-1,1,1,4,4,4-hexafluoro-2-butene, Z-1,1,1,4,4,4-hexafluoro-2-butene, perfluoroethyl perfluoroisopropyl ketone (F-ethyl isopropyl ketone), E-1,3,4,4,4-pentafluoro-3-(trifluoromethyl)-1-butene), E-1,2,3,3,3-pentafluoropropene, Z-1,2,3,3,3-pentafluoropropene, E-1-chloro-2,3,3,3-tetrafluoropropene, Z-1-chloro-2,3,3,3-tetrafluoropropene, CF₃I, carbon dioxide, nitrogen, and argon.

76. (canceled)

77. A process for extinguishing or suppressing a flame comprising dispensing the composition of claim 75 at said flame.

78. (canceled)

79. A method of reducing the flammability of a fluid comprising adding the composition of claim 75 to the fluid.

80-83. (canceled)

84. A sprayable composition comprising a propellant component and a co-propellant component comprising a compound of Formula (I): [chemical structure]
wherein:
R¹ and R⁴ are each independently H, Cl, F, Br, I, a partially fluorinated C₁₋₄ alkoxy, or a perfluorinated C₁₋₄ alkoxy; and
R² is selected from H, Cl, F, Br, I, a partially fluorinated C₁₋₁₀ alkyl, a perfluorinated C₁₋₁₀ alkyl, a partially fluorinated C₁₋₄ alkoxy, and a perfluorinated C₁₋₄ alkoxy;
wherein at least one of R¹, R², and R⁴ is not H;
R³ is selected from partially fluorinated C₁₋₁₀ alkyl and perfluorinated C₁₋₁₀ alkyl;
or alternatively, R² and R³ are each independently selected from partially fluorinated or perfluorinated C₁₋₅ alkylene, which together form a monocyclic ring.

85-90. (canceled)

91. A method of anesthetizing a subject, comprising administering to the subject an effective amount of a compound of Formula (I): [chemical structure]
wherein:
R¹ and R⁴ are each independently H, Cl, F, Br, I, a partially fluorinated C₁₋₄ alkoxy, or a perfluorinated C₁₋₄ alkoxy; and
R² is selected from H, Cl, F, Br, I, a partially fluorinated C₁₋₁₀ alkyl, a perfluorinated C₁₋₁₀ alkyl, a partially fluorinated C₁₋₄ alkoxy, and a perfluorinated C₁₋₄ alkoxy;
wherein at least one of R¹, R², and R⁴ is not H;
R³ is selected from partially fluorinated C₁₋₁₀ alkyl and perfluorinated C₁₋₁₀ alkyl;
or alternatively, R² and R³ are each independently selected from partially fluorinated or perfluorinated C₁₋₅ alkylene, which together form a monocyclic ring.

92. (canceled)
